(19) United States
(12) Patent Application Publication
(10) Pub. No.: US 2025/0368651 A1
(43) Pub. Date: Dec. 4, 2025

(54) ANTIVIRAL COMPOUNDS USEFUL AGAINST SARS-COV-2

(71) Applicant: UNIVERSITY OF KANSAS, Lawrence, KS (US)

(72) Inventors: Jingxin WANG, Lawrence, KS (US); Zhichao TANG, Lawrence, KS (US)

(73) Assignee: UNIVERSITY OF KANSAS, Lawrence, KS (US)

(21) Appl. No.: 18/877,434

(22) PCT Filed: Jun. 27, 2023
(86) PCT No.: PCT/US2023/026356

§ 371 Date: Dec. 20, 2024

Related U.S. Application Data
(60) Provisional application No. 63/355,874, filed on Jun. 27, 2022.

(51) Int. Cl. C07D 487/04; A61K 31/496; A61K 31/4985; A61K 31/519; A61K 31/7076; A61P 31/14; C07D 405/04; C07D 471/04; C07D 519/00; C07H 19/20

(52) U.S. Cl. CPC C07D 487/04; A61K 31/496; A61K 31/4985; A61K 31/519; A61K 31/7076; A61P 31/14; C07D 405/04; C07D 471/04; C07D 519/00; C07H 19/20

(57) ABSTRACT

Disclosed herein are compound of Formula I, Formula II, or Formula III: or a pharmaceutically acceptable salt and/or solvate of any one or more thereof, pharmaceutical compositions including such compounds, and methods of treating disease by administering or contacting a subject with one or more of the above compounds.

### CROSS-REFERENCE TO RELATED APPLICATION

This application claims the benefit of and priority to U.S. Provisional Patent Application No. 63/355,874, filed Jun. 27, 2022, which is incorporated by reference herein in its entirety for any and all purposes.

### STATEMENT REGARDING FEDERALLY FUNDED RESEARCH

This invention was made with government support under GM147498 awarded by the National Institutes of Health. The government has certain rights in the invention.

### TECHNICAL FIELD

The present technology relates generally to antiviral compounds useful against SARS-CoV-2.

### SUMMARY

In an aspect, the present technology provides a compound of Formula I:

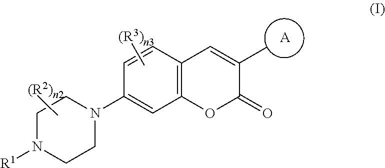

or a pharmaceutically acceptable salt and/or solvate thereof, wherein Ring A is C₆-C₁₀ aryl or 5 to 10 membered heteroaryl; R¹ is selected from H, optionally substituted C₁-C₆ alkyl, —CH₂CCH, or optionally substituted 2 to 15 membered heteroalkyl; each R² is independently halogen or optionally substituted C₁-C₆ alkyl; each R³ is independently selected from halogen, optionally substituted C₁-C₆ alkyl, or optionally substituted 2 to 6 membered heteroalkyl; n2 is 0, 1, 2, 3, 4, or 5; and n3 is 0, 1, 2, 3, or 4; with the proviso that the compound is not: (S)-3-(6,8-dimethylimidazo[1,2-a]pyrazin-2-yl)-7-(4-ethyl-3-methylpiperazin-1-yl)-2H-chromen-2-one (C2); (S)-3-(6,8-dimethylimidazo[1,2-a]pyrazin-2-yl)-7-(3-methylpiperazin-1-yl)-2H-chromen-2-one (C4); tert-butyl (S)-(2-(4-(3-(6,8-dimethylimidazo[1,2-a]pyrazin-2-yl)-2-oxo-2H-chromen-7-yl)-2-methylpiperazin-1-yl)ethyl)carbamate (C6); 3-(3,4-dimethoxyphenyl)-7-(4-methylpiperazin-1-yl)-2H-chromen-2-one (C27); 3-(imidazo[1,2-a]pyridin-2-yl)-7-(piperazin-1-yl)-2H-chromen-2-one (C29); 3-(6-fluoroimidazo[1,2-a]pyridin-2-yl)-7-(piperazin-1-yl)-2H-chromen-2-one (C31); 3-(6-chloroimidazo[1,2-a]pyridin-2-yl)-7-(piperazin-1-yl)-2H-chromen-2-one (C33); or 7-(piperazin-1-yl)-3-(7-(trifluoromethyl)imidazo[1,2-a]pyridin-2-yl)-2H-chromen-2-one (C36).

In another aspect, the present technology provides a compound of Formula II:

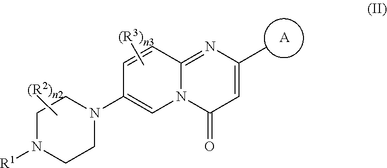

or a pharmaceutically acceptable salt and/or solvate thereof, wherein Ring A is C₆-C₁₀ aryl or 5 to 10 membered heteroaryl; R¹ is selected from H, optionally substituted C₁-C₆ alkyl, —CH₂CCH, or optionally substituted 2 to 15 membered heteroalkyl; each R² is independently halogen or optionally substituted C₁-C₆ alkyl; each R³ is independently selected from halogen, optionally substituted C₁-C₆ alkyl, or optionally substituted 2 to 6 membered heteroalkyl; n2 is 0, 1, 2, 3, 4, or 5; and n3 is 0, 1, 2, 3, or 4; with the proviso that the compound is not: 2-(4,6-dimethylpyrazolo[1,5-a]pyrazin-2-yl)-7-(4-ethylpiperazin-1-yl)-9-methyl-4H-pyrido[1,2-a]pyrimidin-4-one (C3); or 2-(8-fluoro-2-methylimidazo[1,2-a]pyridin-6-yl)-7-(4-methylpiperazin-1-yl)-4H-pyrido[1,2-a]pyrimidin-4-one (C5).

In another aspect, the present technology provides a compound of Formula III:

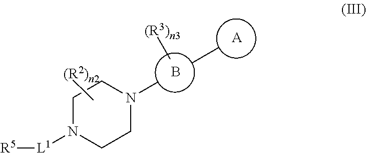

or a pharmaceutically acceptable salt and/or solvate thereof, wherein Ring A is C₆-C₁₀ aryl or 5 to 10 membered heteroaryl; Ring B is 5 to 10 membered heteroaryl; L¹ is

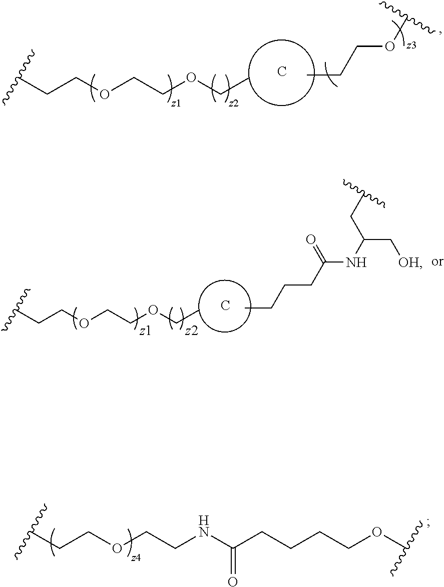

each R² is independently halogen or optionally substituted C₁-C₆ alkyl; each R³ is independently selected from halogen, optionally substituted C₁-C₆ alkyl, or optionally substituted 2 to 6 membered heteroalkyl; R⁵ is

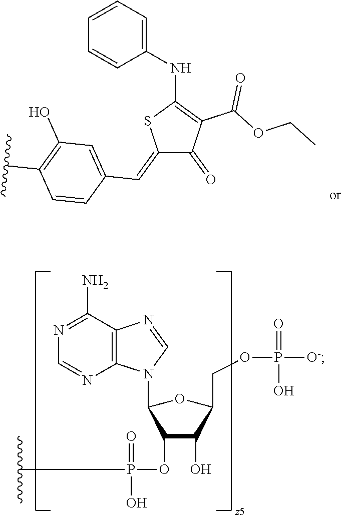

Ring C is a 5-membered heteroaryl; n2 is 0, 1, 2, 3, 4, or 5; n3 is 0, 1, 2, 3, or 4; z1, z3, and z4 are each independently 0, 1, 2, 3 or 4; z2 is independently at each occurrence 1 or 2; and z5 is 1, 2, 3, 4, 5, 6, 7, 8, 9, or 10.

In another aspect, the present technology provides a compound selected from the group consisting of.

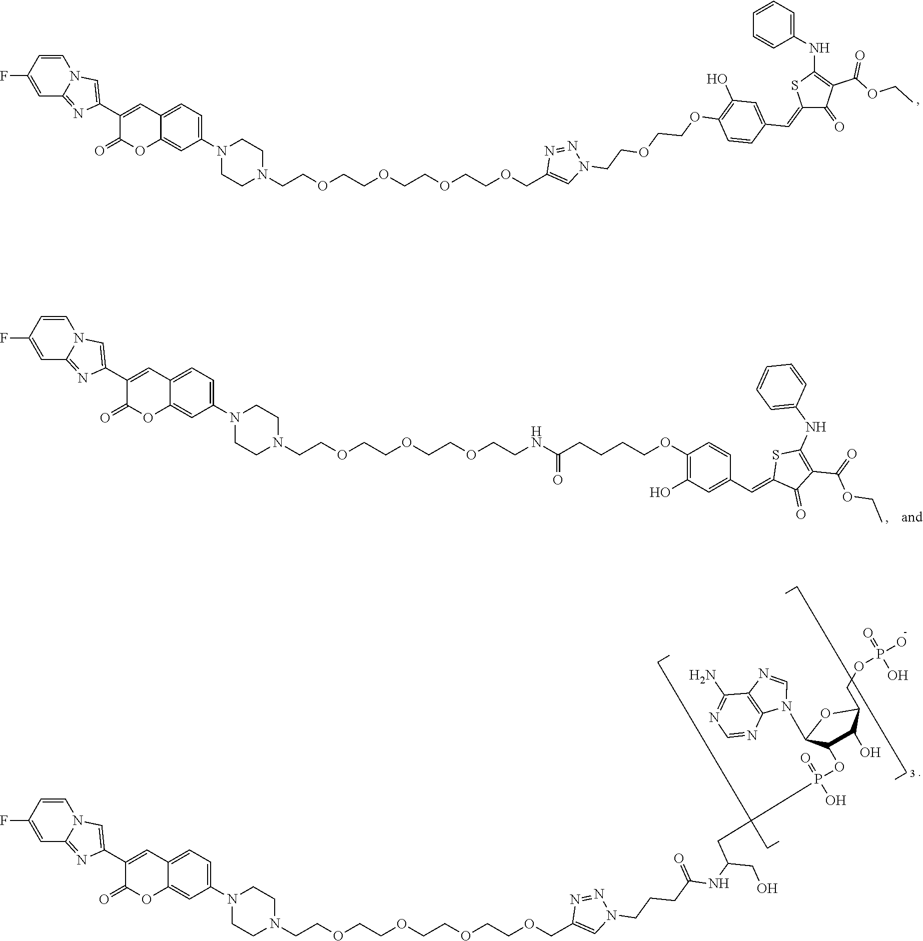

In another aspect, the present technology provides a compound of Formula IV:

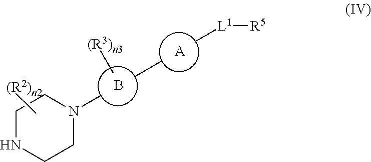

or a pharmaceutically acceptable salt and/or solvate thereof, wherein Ring A is C₆-C₁₀ aryl or 5 to 10 membered heteroaryl; Ring B is 5 to 10 membered heteroaryl; L¹ is

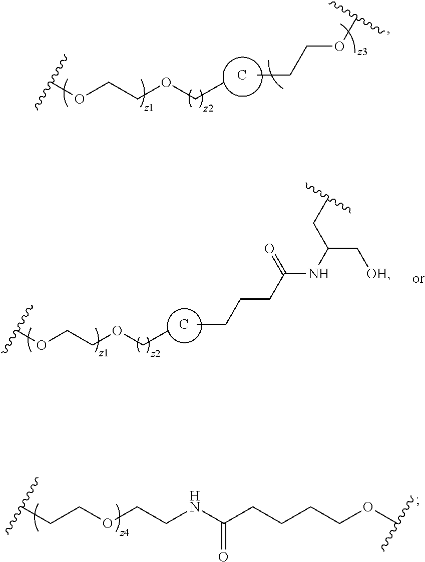

each R² is independently halogen or optionally substituted C₁-C₆ alkyl; each R³ is independently selected from halogen, optionally substituted C₁-C₆ alkyl, or optionally substituted 2 to 6 membered heteroalkyl; R⁵ is

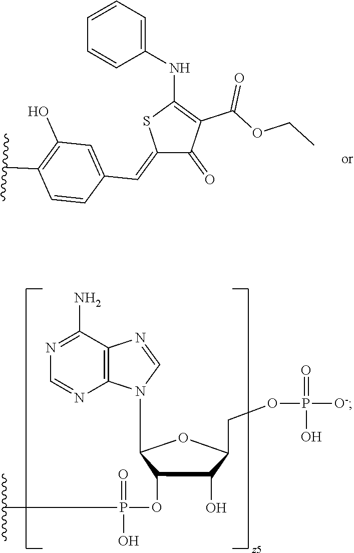

Ring C is a 5-membered heteroaryl; n2 is 0, 1, 2, 3, 4, or 5; n3 is 0, 1, 2, 3 or 4; z1, z3, and z4 are each independently 0, 1, 2, 3, or 4; z2 is independently at each occurrence 1 or 2; and z5 is 1, 2, 3, 4, 5, 6, 7, 8, 9, or 10.

In another aspect, the present technology provides a compound having the following chemical structure:

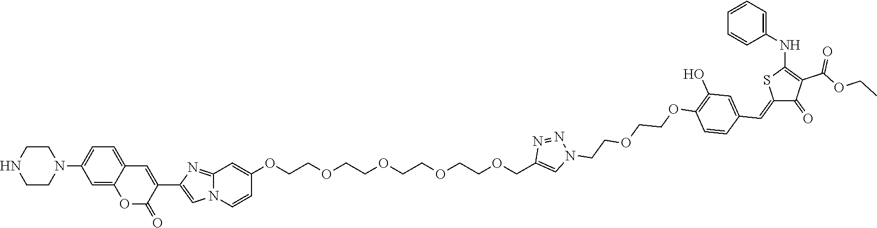 [SMILES: CCOC(=O)C1=C(Nc2ccccc2)S/C(=C\c2ccc(OCCOCCn3cc(COCCOCCOCCOCCOc4ccn5cc(-c6cc7ccc(N8CCNCC8)cc7oc6=O)nc5c4)nn3)c(O)c2)C1=O]

In another aspect, the present technology provides a compound having the following chemical structure:

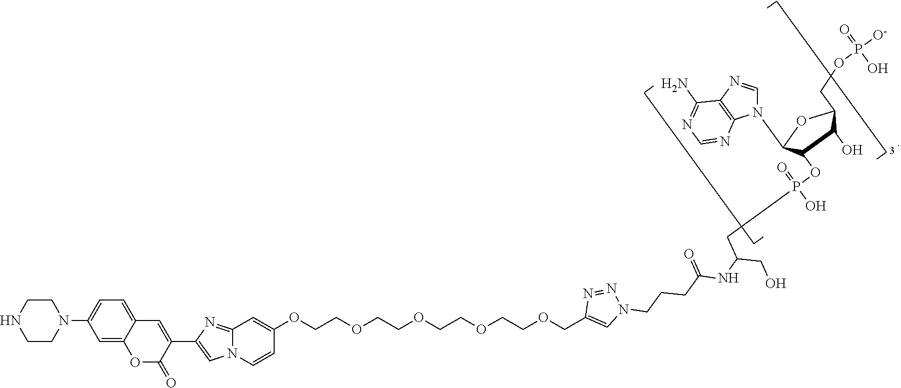

In another aspect, the present technology provides a pharmaceutical composition comprising a compound as disclosed herein, or the stereoisomer or the tautomer thereof, or the pharmaceutically acceptable salt thereof, and at least one pharmaceutically acceptable excipient.

In another aspect, the present technology provides a method of treating a disorder or disease in a subject in need thereof, the method comprising administering to the subject a therapeutically effective amount of a compound as disclosed herein, or the stereoisomer or the tautomer thereof, or the pharmaceutically acceptable salt thereof, or a therapeutically effective amount of a pharmaceutical composition as disclosed herein, wherein the disorder or disease is spinal muscular atrophy (SMA).

In another aspect, the present technology provides a method of treating a disorder or disease in a subject in need thereof, the method comprising administering to the subject a therapeutically effective amount of a compound as disclosed herein, or the stereoisomer or the tautomer thereof, or the pharmaceutically acceptable salt thereof, or a therapeutically effective amount of a pharmaceutical composition as disclosed herein, wherein the disorder or disease is a viral disorder or disease.

### BRIEF DESCRIPTION OF THE DRAWINGS

FIG. 1 shows risdiplam's dual binding sites in exon 7 of SMN2 pre-mRNA. One of the risdiplam analogues, SMN-C5, binds to site 1 by stabilizing a bulged A (exon 7+54) between the 5′-splice site and U1 snRNA and subsequently enhances the U1 snRNP recruitment (17). Ψ=pseudouridine. Terminal stem-loop (TLS2) is an inhibitory cis-acting regulatory element for exon 7 splicing (exonic splicing silencer). The SMN2 pre-mRNA sequence from exon 7+18 to intron 7+10 is shown.

FIGS. 2A-2B. FIG. 2A shows the structures of risdiplam and its active analogues, SMN-C2 and SMN-C5. FIG. 2B shows the fluorescence polarization (FP) of SMN-C2 (0.5 μM) and RNA and DNA sequences (50 μM) of various lengths. NA (−)=no nucleic acid added. All data were reproduced in triplicates. The adjusted P values were calculated by Dunnett's test (ns=not significant). ^{a}In the DNA sequences, deoxyribonucleotides, dA, dT, dG, and dC, were used in substitution of A, U, G, and C in RNA.

FIGS. 3A-3C shows isothermal calorimetry (ITC). FIG. 3A shows raw differential power (DP) and FIG. 3B shows integrated data for SMN-C2 (250 μM) and DNA Seq6 (25 μM) in a buffer that contains 30 mM 2-(N-morpholino)ethanesulfonic acid (MES, pH 6.1), 5% DMSO, and 100 mM NaCl. FIG. 3C shows size-exclusion chromatography with a Superdex 75 column for an annealed mixture of Seq4 (100 μM), Seq4_RC (50 μM), and SMN-C2 (100 μM) 1× phosphate buffered saline (PBS) with the absorption (black, absorption at 280 nm for DNA) and fluorescence readout (fluorescence at excitation/emission=410/480 nm for SMN-C2). Excess amount of SMN-C2 was eluted at retention volume ˜21 mL. The figures are the representation of three independent experiments.

FIG. 4 shows GaMD simulations revealed spontaneous binding of compound 1 to RNA Seq6 and representative conformation of compound 1-bound RNA Seq6 in the folded state. Black=compound 1, Light grey=RNA Seq6, dashed line=polar interaction.

FIGS. 5A-5B. FIG. 5A shows ¹H NMR titration of compound 1 (100 μM) with DNA Seq4 at various concentrations, from top to bottom: 0 mol % (no DNA), 2.5 mol % DNA, 5 mol % DNA, 10 mol % DNA, 20 mol % DNA, and a control spectrum of DNA (20 μM) without compound 1. FIG. 5B shows the numbering of compound 1 and the relative saturation-transfer difference (STD) at different position of compound 1 in a mixture of DNA (0.5 μM) and compound 1 (10 μM) solution. STD was not detected (N.D.) in the whole piperazine ring.

FIG. 6 shows various RNA or DNA secondary structures that harbour the SMN-C2 putative binding site (highlighted in red). ^{a}In DNA sequences, deoxyribonucleotides, dA, dT, dG, and dC, were used in substitution of ribonucleotides A, U, G, and C for RNA. ^{b}Only DNA sequences were investigated. The K_{d} range is the 95% confidence interval of the calculated 50% response concentration from the Sigmoidal 4PL interpolation. All dose titrations were reproduced in three replicates. N.T.=not tested.

FIGS. 7A-7D. FIG. 7A shows the gene assembly of the SARS-CoV-2 (RefSeq NC_045512). ORF1b is continuously synthesized after ORF1a when PFS occurs. Nsps are produced by viral protease cleavage from pp1a and pp1ab. Nsp1=leader protein, nsp3 and 5=proteases, nsp12=RdRp, nsp13=helicase, nsp14=3′-to-5′ exonuclease, nsp15=endo-RNase, nsp16=2′-O-ribose methyltransferase. Transcription regulatory sequence (TRS) and C2NH binding sequences (GRNGGANRG, R=A/G) are depicted. FIG. 7B shows the mutation rate in the SL5 region. Sequences 1-2 are SARS-CoV and SARS-CoV-2 RefSeq. Sequences 3-7 are the recent mutant strains isolated in the Philippines (1/16/2021), USA (3/16/2021), Ghana (1/10/2021), Italy (1/18/2021), and Ghana (1/13/2021), respectively. Dash box=nucleotides at the four-helix junction. FIG. 7C shows the RNA structure of SL5 and nearby regions. FIG. 7D shows the deconvolution of the RNA-binding site within SL5. C30 selectively binds to the RNA structure containing the bulged G in SL5.

FIGS. 8A-8B. FIG. 8A shows structures of SMN-C2 and the optimization of ring E for SL5 binding. The dissociation constants were listed in the table. FIG. 8B shows titration of fluorescence polarization for SARS-CoV-2 RNA SL5 and SMN-C2, C29, and C30.

FIGS. 9A-9B shows the dose-response curves in the in vitro fluorescence polarization (FP) binding assay.

FIG. 10 shows the fluorescence polarization (FP) assay dose-response curves for sequences with K_{d} values in Table 1, plotted using GraphPad Prism 8.

FIG. 11 shows the fluorescence polarization (FP) assay dose-response curves for compounds with K_{d} values in Table 2, plotted using GraphPad Prism 8.

FIG. 12 shows the fluorescence polarization (FP) assay dose-response curves for sequences with K_{d} values in Scheme 2, plotted using GraphPad Prism 8.

FIG. 13 shows the fluorescence polarization assay dose-response curves for sequences in Table S2, plotted using GraphPad Prism 8.

FIG. 14 shows the fluorescence polarization assay dose-response curves for compounds in Table S4, plotted using GraphPad Prism 8.

FIG. 15 shows cell-based SMN2 splicing assay with minigenes that harbor different mutations in the GA-rich sequence. The minigene-transfected 293T cells were treated with different concentrations of SMN-C2 for 24 h. The EC₅₀ was calculated using the disappearance of the Δexon 7 band. The figure is a representative of three biological replicates.

FIG. 16 shows ITC raw data for SMN-C2 (300 μM) and dsDNA (43 μM, annealed Seq6 and its reverse complement) in a buffer that contains 30 mM 2-(N-morpholino)ethanesulfonic acid (MES, pH 6.1), 5% DMSO, and 100 mM NaCl. The figures are representation of three independent experiments.

FIGS. 17A-17B. FIG. 17A shows size-exclusion chromatography with a Superdex 75 column for DNAs Seq4 (50 μM), Seq4_RC (50 μM), and the dsDNA made by annealing equal molar of the DNAs Seq4 and Seq4_RC. FIG. 17B shows size-exclusion chromatography with a Superdex 75 column for an annealed mixture of Seq4 (100 μM), Seq4_RC (50 μM), and SMN-C2 (100 μM) 1× phosphate buffered saline (PBS) with the absorption (black, absorption at 280 nm for DNA) and fluorescence readout (red, fluorescence at excitation/emission=410/480 nm for SMN-C2). Excess amount of SMN-C2 was eluted at retention volume ˜21 mL. The figures are the representation of three independent experiments.

FIGS. 18A-18E. FIG. 18A shows size-exclusion chromatography with a Superdex 30 column for random 45nt ssDNA, Seq S28=5′-TACAGATCTACTAGTGATCTATGACTGATCTGTACA-TGATCTACA. FIG. 18B shows size-exclusion chromatography with a Superdex 30 column for random 22nt ssDNA, Seq S29=5′-CAGGTGTCCACTCCCAGT-TCAA. FIG. 18C shows size-exclusion chromatography with a Superdex 30 column for reverse complement of Seg19, Seg19_RC=ACCCTCCCTCA. FIG. 18D shows size-exclusion chromatography with a Superdex 30 column for Seq19=5′-TGAGGGAGGGT. FIG. 18E shows size-exclusion chromatography with a Superdex 30 column for a 1:1 annealed mixture of Seg19 and Seg19_RC. All DNA samples were prepared in 1× phosphate buffered saline (PBS) at 100 μM.

FIG. 19 shows size-exclusion chromatography with a Superdex 75 column for RNA Seq11.

FIGS. 20A-20B. FIG. 20A shows the “Intermediate” conformational states of RNA-compound 1 obtained from the GaMD simulations. FIG. 20B shows the “Unbound/Unfolded” conformational states of RNA-compound 1 obtained from the GaMD simulations.

FIGS. 21A-21B. FIG. 21A shows the “Bound/Unfolded” conformational states of DNA Seq6 during binding of compound 1 obtained from the GaMD simulations. FIG. 21B shows the “Intermediate” conformational states of DNA Seq6 during binding of compound 1 obtained from the GaMD simulations.

FIGS. 22A-22C show SPR kinetic evaluation results of (FIG. 22A) SMN-C2, (FIG. 22B) SMN-C3, and (FIG. 22C) SMN-C5 with RNA Seq4.

FIGS. 23A-23D shows circular dichroism of (FIG. 23A) RNA Seq6 and (FIG. 23B) reverse complement of RNA Seq6 (RNA Seq6 RC) in the presence or absence of SMN-C2 (262.5 μM); (FIG. 23C) DNA Seq6 and (FIG. 23D) reverse complement of DNA Seq4 (DNA Seq4 RC) in the presence or absence of compound 1 (60 μM). RNAs and DNAs were prepared at 175 μM, 40 μM respectively in 30 mM HEPES (pH 7.3) and 100 mM NaCl. HEPES=4-(2-hydroxyethyl)-1-piperazineethanesulfonic acid.

FIGS. 24A-24B show the ultraviolet-visible spectroscopy of (FIG. 24A) SMN-C2 (262.5 μM) and RNA Seq6 (175 μM); (FIG. 24B) compound 1 (60 μM) and DNA Seq6 (40 μM) in 30 mM HEPES (pH 7.3) and 100 mM NaCl.

FIG. 25 shows full gel images for FIG. 15.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, B and C, or the combination of A, B, and C.”

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; and nitriles (i.e., CN).

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Heteroalkyl groups include straight chain and branched chain alkyl groups except that at least one carbon atom is replaced by a heteroatom (e.g., oxygen, nitrogen, or sulfur). Heteroalkyl groups typically include from 1 to 15 carbons or, in some embodiments, from 1 to 10, from 1 to 8, 1 to 6, or 1 to 4 carbon atoms. Heteroalkyl groups typically contain 1, 2, 3, 4, or 5 heteroatoms, e.g., 1, 2, 3, 4, or 5 oxygen atoms. Heteroalkyl groups may be substituted or unsubstituted. Examples of straight chain heteroalkyl groups include derivatives of polyethylene glycol.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)=CH₂, —C(CH₃)═CH(CH₃), —C(CH₂CH₃)=CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. The phrase includes heterocyclyl groups that have other groups, such as alkyl, oxo or halo groups, bonded to one of the ring members, referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂₀R⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O—. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

The term “pentafluorosulfanyl” refers to —SF₅.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g. Na⁺, Li⁺, K⁺, Ca²⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

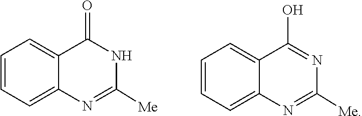 [SMILES: Cc1nc2ccccc2c(=O)[nH]1 ; Cc1nc(O)c2ccccc2n1]

As another example, guanidines may exhibit the following isomeric forms in protic organic solution, also referred to as tautomers of each other:

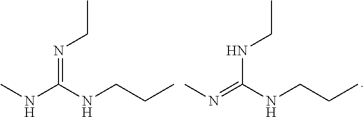

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

### THE PRESENT TECHNOLOGY

In an aspect, the present technology provides a compound of Formula I:

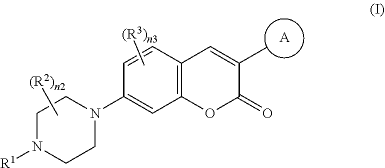

or a pharmaceutically acceptable salt and/or solvate thereof, wherein Ring A is C₆-C₁₀ aryl or 5 to 10 membered heteroaryl; R¹ is selected from H, optionally substituted C₁-C₆ alkyl, —CH₂CCH, or optionally substituted 2 to 15 membered heteroalkyl; each R² is independently halogen or optionally substituted C₁-C₆ alkyl; each R³ is independently selected from halogen, optionally substituted C₁-C₆ alkyl, or optionally substituted 2 to 6 membered heteroalkyl; n2 is 0, 1, 2, 3, 4, or 5; and n3 is 0, 1, 2, 3, or 4; with the proviso that the compound is not: (S)-3-(6,8-dimethylimidazo[1,2-a]pyrazin-2-yl)-7-(4-ethyl-3-methylpiperazin-1-yl)-2H-chromen-2-one (C2); (S)-3-(6,8-dimethylimidazo[1,2-a]pyrazin-2-yl)-7-(3-methylpiperazin-1-yl)-2H-chromen-2-one (C4); tert-butyl (S)-(2-(4-(3-(6,8-dimethylimidazo[1,2-a]pyrazin-2-yl)-2-oxo-2H-chromen-7-yl)-2-methylpiperazin-1-yl)ethyl)carbamate (C6); 3-(3,4-dimethoxyphenyl)-7-(4-methylpiperazin-1-yl)-2H-chromen-2-one (C27); 3-(imidazo[1,2-a]pyridin-2-yl)-7-(piperazin-1-yl)-2H-chromen-2-one (C29); 3-(6-fluoroimidazo[1,2-a]pyridin-2-yl)-7-(piperazin-1-yl)-2H-chromen-2-one (C31); 3-(6-chloroimidazo[1,2-a]pyridin-2-yl)-7-(piperazin-1-yl)-2H-chromen-2-one (C33); or 7-(piperazin-1-yl)-3-(7-(trifluoromethyl)imidazo[1,2-a]pyridin-2-yl)-2H-chromen-2-one (C36).

In another aspect, the present technology provides a compound of Formula II:

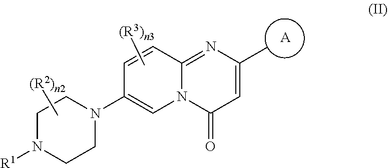

or a pharmaceutically acceptable salt and/or solvate thereof, wherein Ring A is C₆-C₁₀ aryl or 5 to 10 membered heteroaryl; R¹ is selected from H, optionally substituted C₁-C₆ alkyl, —CH₂CCH, or optionally substituted 2 to 15 membered heteroalkyl; each R² is independently halogen or optionally substituted C₁-C₆ alkyl; each R³ is independently selected from halogen, optionally substituted C₁-C₆ alkyl, or optionally substituted 2 to 6 membered heteroalkyl; n2 is 0, 1, 2, 3, 4, or 5; and n3 is 0, 1, 2, 3, or 4; with the proviso that the compound is not: 2-(4,6-dimethylpyrazolo[1,5-a]pyrazin-2-yl)-7-(4-ethylpiperazin-1-yl)-9-methyl-4H-pyrido[1,2-a]pyrimidin-4-one (C3); or 2-(8-fluoro-2-methylimidazo[1,2-a]pyridin-6-yl)-7-(4-methylpiperazin-1-yl)-4H-pyrido[1,2-a]pyrimidin-4-one (C5).

In embodiments, Ring A is substituted or unsubstituted phenyl, naphthyl, quinolyl, imidazopyrazinyl, or imidazopyridinyl. In embodiments, Ring A is phenyl, naphthyl, quinolyl, imidazopyrazinyl, or imidazopyridinyl each optionally substituted with 1 to 3 —F, —Cl, —Br, —CH₃, —CH₂CH₃, —CF₃, —OCH₃, —OCH₂CH₃, or —OCF₃.

In embodiments, each R¹ is independently H, —CH₃, or —CH₂CH₃. In embodiments, each R¹ is

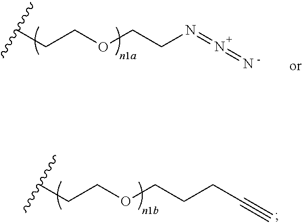

wherein n1a and n1b are independently selected from 1, 2, 3, 4, or 5.

In embodiments, each R² is independently hydrogen, —CH₃, or —CH₂CH₃. In embodiments, each R² is independently halogen, —CH₃, or —CH₂CH₃.

In embodiments, each R³ is independently —F, —Cl, —Br, —CH₃, or —CH₂CH₃. In embodiments, each R³ is independently —O(CH₂)₂NHC(O)O-t-butyl or —O(CH₂)₂NHC(O)OH.

In embodiments, n2 is 0 or 1. In embodiments, n3 is 0 or 1.

In embodiments, the present technology provides a compound selected from the group consisting of:

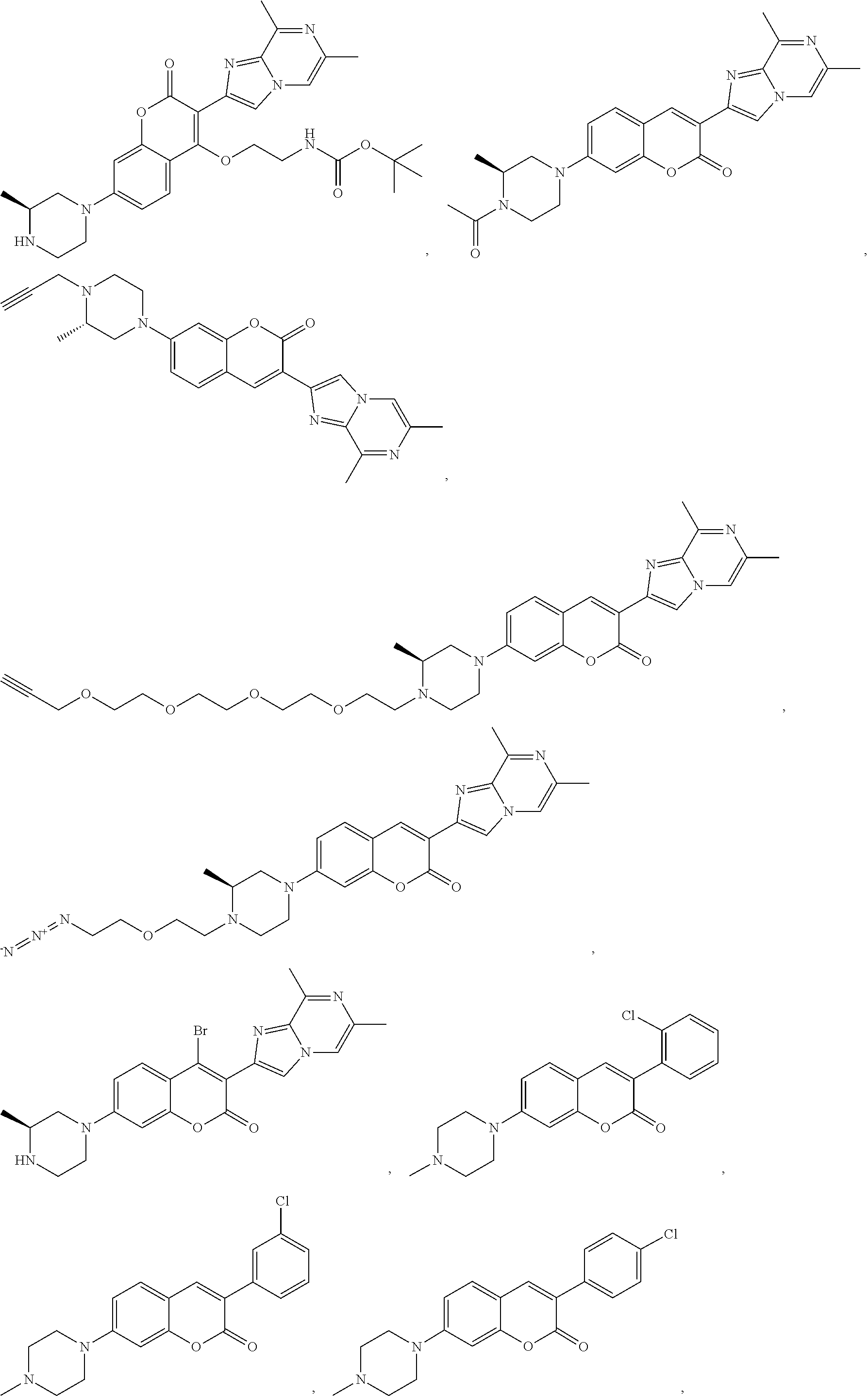

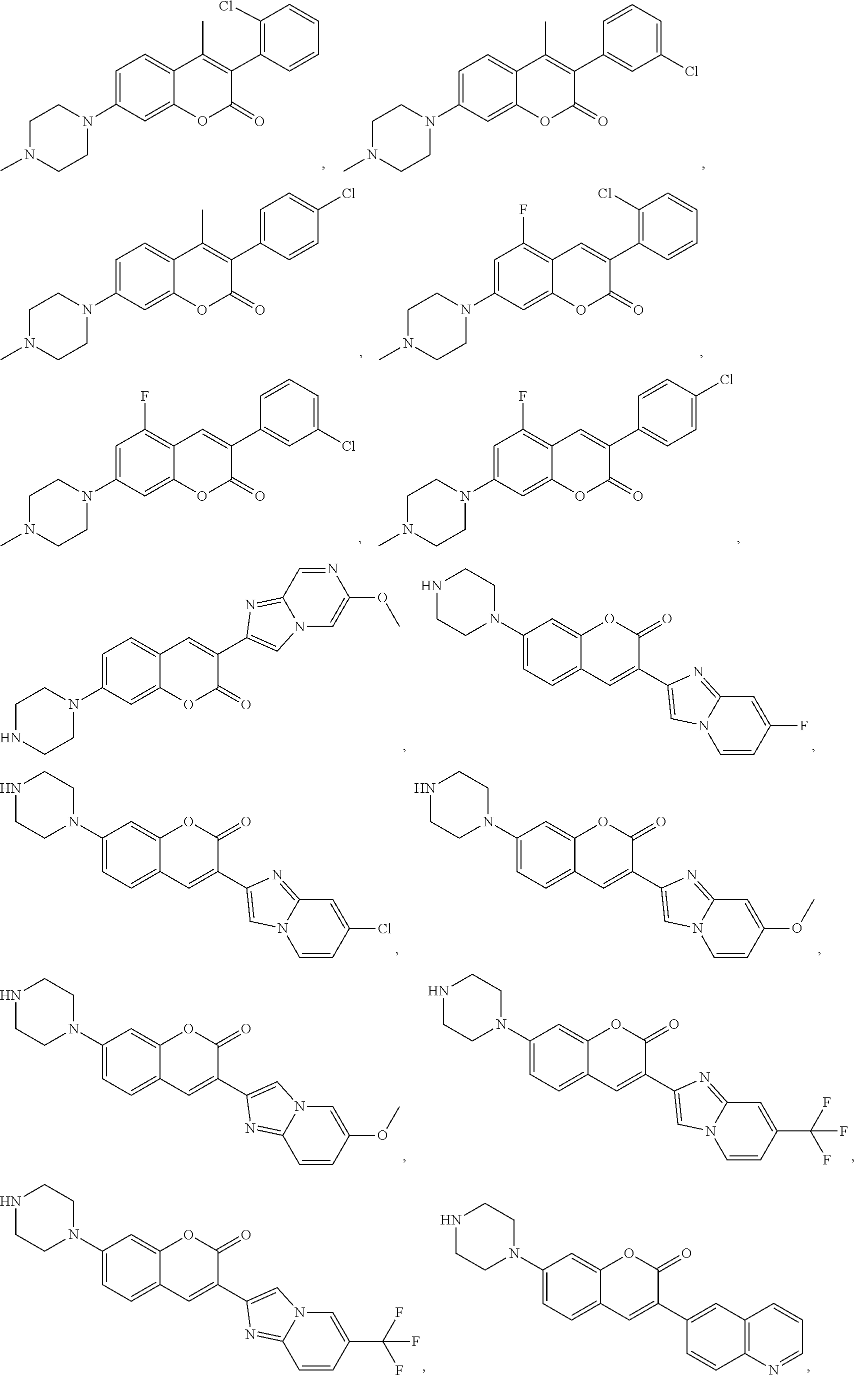

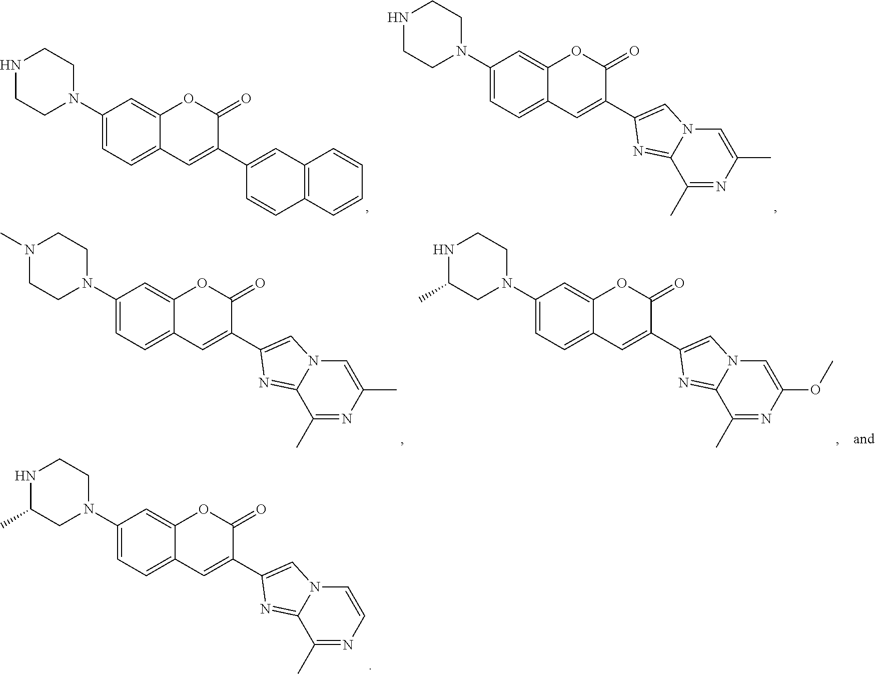

In embodiments, the present technology provides a compound selected from the group consisting of:

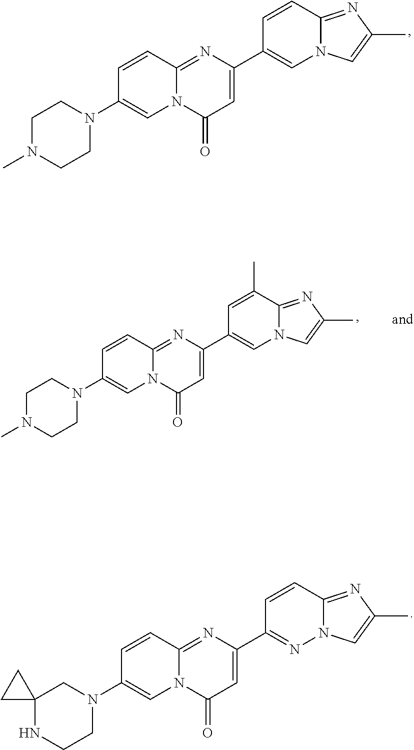

In another aspect, the present technology provides a compound of Formula III:

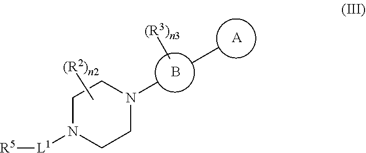

or a pharmaceutically acceptable salt and/or solvate thereof, wherein Ring A is C₆-C₁₀ aryl or 5 to 10 membered heteroaryl; Ring B is 5 to 10 membered heteroaryl; L¹ is

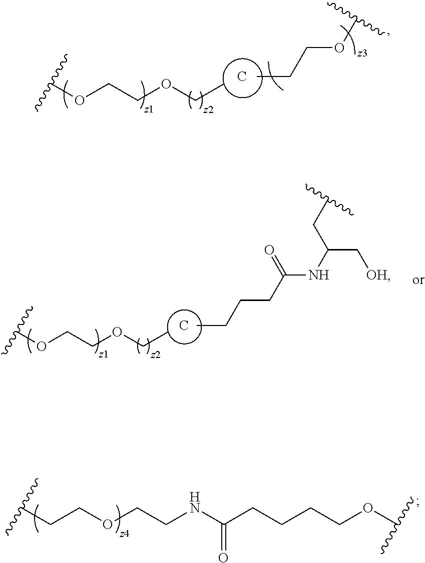

each R² is independently halogen or optionally substituted C₁-C₆ alkyl; each R³ is independently selected from halogen, optionally substituted C₁-C₆ alkyl, or optionally substituted 2 to 6 membered heteroalkyl; R is

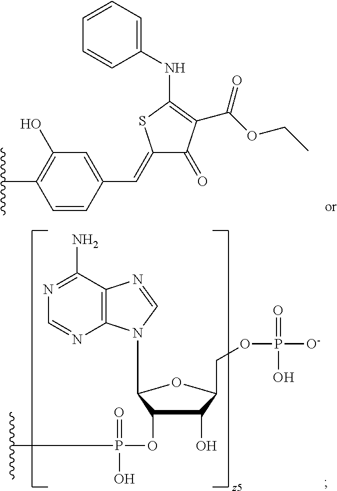

Ring C is a 5-membered heteroaryl; n2 is 0, 1, 2, 3, 4, or 5; n3 is 0, 1, 2, 3, or 4; z1, z3, and z4 are each independently 0, 1, 2, 3, or 4; z2 is independently at each occurrence 1 or 2; and z5 is 1, 2, 3, 4, 5, 6, 7, 8, 9, or 10. In embodiments, z5 is 3, 4, 5, 6, 7, or 8. In embodiments, z5 is 3.

In embodiments, the compound has the chemical structure of Formula IIIa:

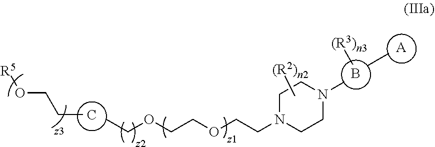

or a pharmaceutically acceptable salt and/or solvate thereof.

In embodiments, the compound has the chemical structure of Formula IIIb:

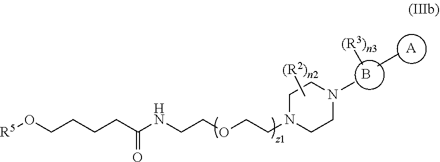

or a pharmaceutically acceptable salt and/or solvate thereof.

In embodiments, the compound has the chemical structure of Formula IIIc:

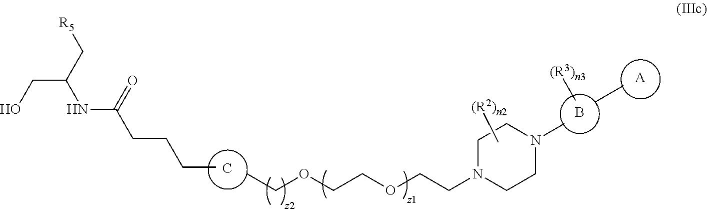

or a pharmaceutically acceptable salt and/or solvate thereof.

In embodiments, Ring B is selected from coumarin and pyridopyrimidone.

In embodiments, the compound has the chemical structure of Formula IIIa′:

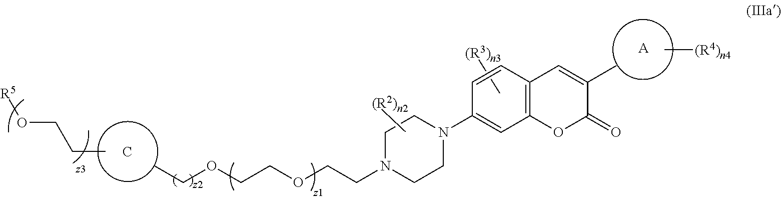

or a pharmaceutically acceptable salt and/or solvate thereof.

In embodiments, the compound has the chemical structure of Formula IIIb′:

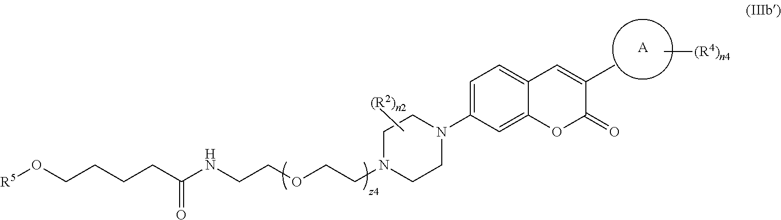

or a pharmaceutically acceptable salt and/or solvate thereof.

In embodiments, the compound has the chemical structure of Formula IIIc′:

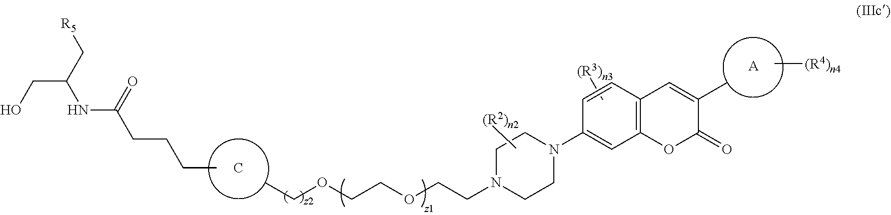

or a pharmaceutically acceptable salt and/or solvate thereof.

In embodiments, Ring C is 1,2,3-triazinyl.

In embodiments, Ring A is substituted or unsubstituted phenyl, naphthyl, quinolyl, imidazopyrazinyl, or imidazopyridinyl. In embodiments, Ring A is phenyl, naphthyl, quinolyl, imidazopyrazinyl, or imidazopyridinyl each optionally substituted with 1 to 3 —F, —Cl, —Br, —CH₃, —CH₂CH₃, —CF₃, —OCH₃, —OCH₂CH₃, or —OCF₃

In embodiments, each R² is independently hydrogen, —CH₃, or —CH₂CH₃. In embodiments, each R² is independently halogen, —CH₃, or —CH₂CH₃

In embodiments, each R³ is independently —F, —Cl, —Br, —CH₃, or —CH₂CH₃. In embodiments, each R³ is independently —O(CH₂)₂NHC(O)O-t-butyl or —O(CH₂)₂NHC(O)OH.

In embodiments, n2 is 0 or 1.

In embodiments, n3 is 0 or 1.

In embodiments, z5 is 3, 4, 5, 6, 7, or 8.

In embodiments, z5 is 3.

In another aspect, the present technology provides a compound selected from the group consisting of:

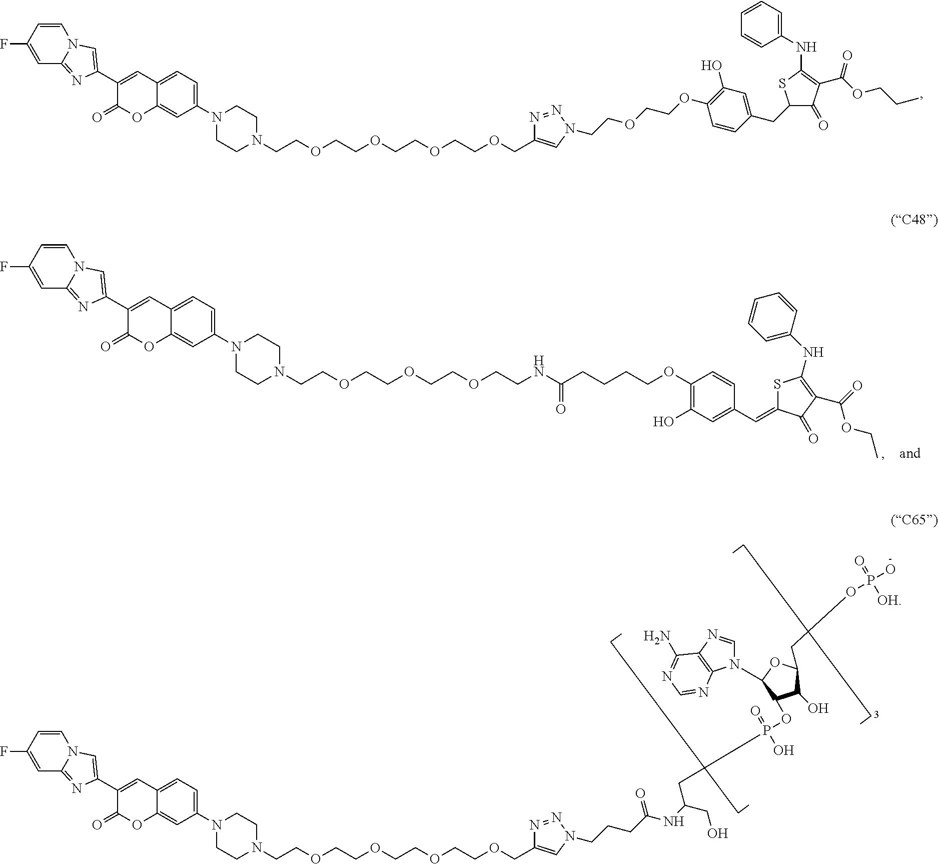

In another aspect, the present technology provides a compound of Formula IV:

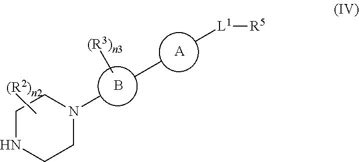

or a pharmaceutically acceptable salt and/or solvate thereof, wherein Ring A is C₆-C₁₀ aryl or 5 to 10 membered heteroaryl; Ring B is 5 to 10 membered heteroaryl; L¹ is

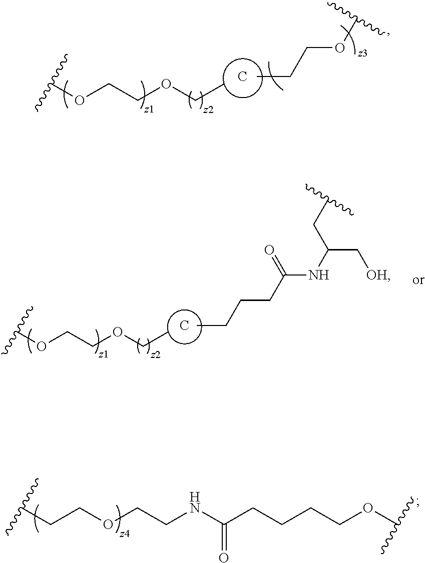

each R² is independently halogen or optionally substituted C₁-C₆ alkyl; each R³ is independently selected from halogen, optionally substituted C₁-C₆ alkyl, or optionally substituted 2 to 6 membered heteroalkyl; R⁵ is

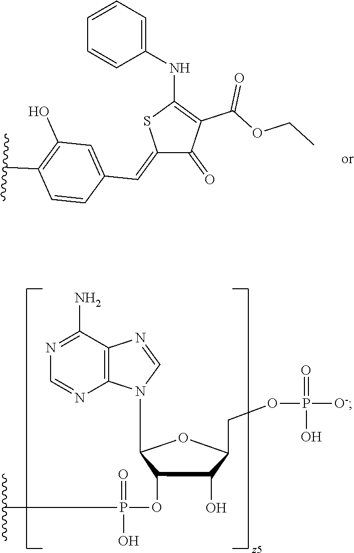

Ring C is a 5-membered heteroaryl; n2 is 0, 1, 2, 3, 4, or 5; n3 is 0, 1, 2, 3 or 4; z1, z3, and z4 are each independently 0, 1, 2, 3, or 4; z2 is independently at each occurrence 1 or 2; and z5 is 1, 2, 3, 4, 5, 6, 7, 8, 9, or 10.

In some embodiments, the compound has the chemical structure of Formula IVa:

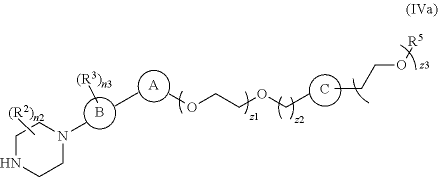

or a pharmaceutically acceptable salt and/or solvate thereof.

In some embodiments, the compound has the chemical structure of Formula IVb:

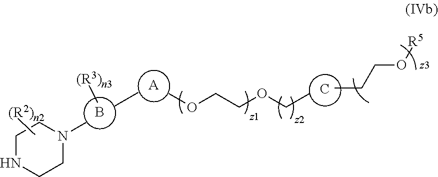

or a pharmaceutically acceptable salt and/or solvate thereof.

In some embodiments, Ring B is selected from coumarin and pyridopyrimidone.

In some embodiments, the compound has the chemical structure of Formula IVa′:

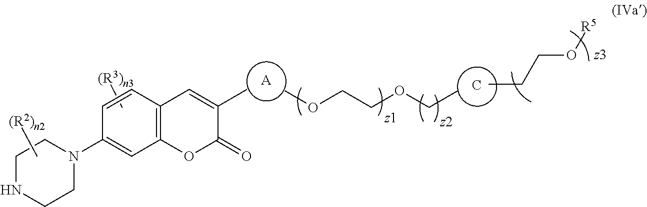

or a pharmaceutically acceptable salt and/or solvate thereof.

In some embodiments, the compound has the chemical structure of Formula IVb′.

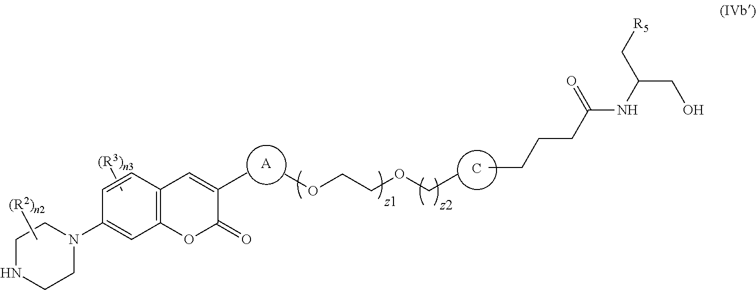

or a pharmaceutically acceptable salt and/or solvate thereof.

In some embodiments, Ring C is 1,2,3-triazinyl,

In some embodiments, Ring A is substituted or unsubstituted phenyl, naphthyl, quinolyl, imidazopyrazinyl, or imidazopyridinyl.

In some embodiments, each R² is independently hydrogen, —CH₃, or —CH₂CH₃.

In some embodiments, each R³ is independently —F, —Cl, —Br, CH₃, or CH₂CH₃. In some embodiments, each R³ is independently —O(CH₂)₂NHC(O)O-t-butyl or —O(CH₂)₂NHC(O)OH.

In some embodiments, Ring A is phenyl, naphthyl, quinolyl, imidazopyrazinyl, or imidazopyridinyl each optionally substituted with 1 to 3 —F, —Cl, —Br, —CH₃, —CH₂CH₃, —CF₃, —OCH₃, —OCH₂CH₃, or —OCF₃.

In some embodiments, n2 is 0. In some embodiments, n2 is 1. In some embodiments, n3 is 0. In some embodiments, n3 is 1.

In some embodiments, z5 is 3, 4, 5, 6, 7, or 8. In some embodiments, z5 is 3.

In another aspect the present technology provides a compound having the following chemical structure:

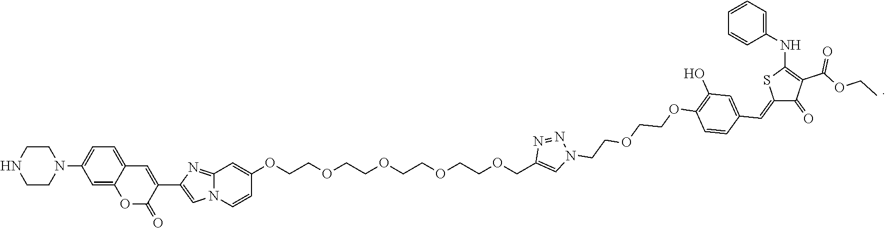 [SMILES: CCOC(=O)C1=C(Nc2ccccc2)S/C(=C\c2ccc(OCCOCCn3cc(COCCOCCOCCOCCOc4ccn5cc(-c6cc7ccc(N8CCNCC8)cc7oc6=O)nc5c4)nn3)c(O)c2)C1=O]

In another aspect the present technology provides a compound having the following chemical structure:

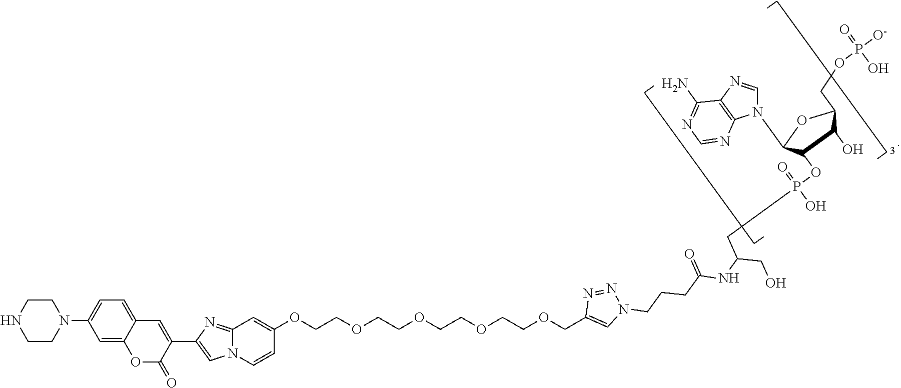

In an aspect, a composition is provided that includes any one of the herein-described embodiments of compounds of Formula I, Formula II, Formula III, Formula IIIa, Formula IIIb, Formula IIIc, Formula IIIa′, Formula IIIb′, Formula IIIc′, Formula IV, Formula IVa, Formula IVb, Formula IVa′, or Formula IVb′ and also includes a pharmaceutically acceptable carrier. In any embodiment herein, it may be that a compound of the present technology is part of a pharmaceutical composition, the pharmaceutical composition including an effective amount of the compound of any one of the aspects and embodiments of compounds of Formula I and a pharmaceutically acceptable carrier.

In another aspect, the present technology provides a pharmaceutical composition comprising a compound as disclosed herein, or the stereoisomer or the tautomer thereof, or the pharmaceutically acceptable salt thereof, and at least one pharmaceutically acceptable excipient.

In another aspect, the present technology provides a method of treating a disorder or disease in a subject in need thereof, the method comprising administering to the subject a therapeutically effective amount of a compound as disclosed herein, or the stereoisomer or the tautomer thereof, or the pharmaceutically acceptable salt thereof, or a therapeutically effective amount of a pharmaceutical composition as disclosed herein, wherein the disorder or disease is spinal muscular atrophy (SMA).

In another aspect, the present technology provides a method of treating a disorder or disease in a subject in need thereof, the method comprising administering to the subject a therapeutically effective amount of a compound as disclosed herein, or the stereoisomer or the tautomer thereof, or the pharmaceutically acceptable salt thereof, or a therapeutically effective amount of a pharmaceutical composition as disclosed herein, wherein the disorder or disease is a viral disorder or disease.

“Effective amount” refers to the amount of a compound or composition required to produce a desired effect. One example of an effective amount includes amounts or dosages that yield acceptable toxicity and bioavailability levels for therapeutic (pharmaceutical) use including, but not limited to, the treatment of a bacterial infection. As used herein, a “subject” or “patient” is a mammal, such as a cat, dog, rodent or primate. Typically the subject is a human, and, preferably, a human suffering from or suspected of suffering from pain. The term “subject” and “patient” can be used interchangeably.

Thus, the instant present technology provides pharmaceutical compositions and medicaments comprising any of the compounds disclosed herein (e.g., compounds of Formula I) and a pharmaceutically acceptable carrier or one or more excipients or fillers. The compositions may be used in the methods and treatments described herein. Such compositions and medicaments include a therapeutically effective amount of any compound as described herein, including but not limited to a compound of Formula I. The pharmaceutical composition may be packaged in unit dosage form. The unit dosage form is effective in treating a bacterial infection when administered to a subject in need thereof.

The pharmaceutical compositions and medicaments may be prepared by mixing one or more compounds of the present technology, pharmaceutically acceptable salts thereof, stereoisomers thereof, tautomers thereof, or solvates thereof, with pharmaceutically acceptable carriers, excipients, binders, diluents or the like. The compounds and compositions described herein may be used to prepare formulations and medicaments that prevent or treat a bacterial infection. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until, for example, culture of the bacterial infection indicates a reduction in the number of bacteria and/or the symptoms of the bacterial infection decrease (e.g., as indicated by the patient). The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day may be sufficient (e.g., a dosage in the range of about 0.01 to about 10 mg per kg of body weight per day may be sufficient). The specific dosage used, however, can vary or may be adjusted as considered appropriate by those of ordinary skill in the art. For example, the dosage can depend on a number of factors including the requirements of the patient, the severity of the bacterial infection and the pharmacological activity of the compound being used. The determination of optimum dosages for a particular patient is well known to those skilled in the art.

Various assays and model systems can be readily employed to determine the therapeutic effectiveness of the treatment according to the present technology. Effectiveness of the compositions and methods of the present technology may also be demonstrated by a culture of the bacterial infection indicating a reduction in the number of bacteria subsequent to administering a compound and/or composition of the present technology and/or the symptoms of the bacterial infection decrease (e.g., as indicated by the patient).

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

As indicated earlier in this disclosure, the compounds of the present technology can also be administered to a patient along with other conventional therapeutic agents that may be useful in the treatment of a bacterial infection, such as a fluoroquinolone antibiotic, an aminoglycoside antibiotic, and/or a polymyxin antibiotic. In any embodiment herein, a compound and/or composition of the present technology may be administered along with an effective amount of a fluoroquinolone antibiotic, an effective amount of a aminoglycoside antibiotic, and/or a polymyxin antibiotic. The administration may include oral administration, parenteral administration, nasal administration, or topical administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent (e.g., an antiviral drug or drug for treating COVID-19 (e.g. remdesivir)) in an amount that can potentially or synergistically be effective for the treatment of a viral disease or disorder (e.g., COVID-19).

In an aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

A compound of the present technology can also be modified, for example, by the covalent attachment of an organic moiety or conjugate to improve pharmacokinetic properties, toxicity or bioavailability (e.g., increased in vivo half-life). The conjugate can be a linear or branched hydrophilic polymeric group, fatty acid group or fatty acid ester group. A polymeric group can comprise a molecular weight that can be adjusted by one of ordinary skill in the art to improve, for example, pharmacokinetic properties, toxicity or bioavailability. Exemplary conjugates can include a polyalkane glycol (e.g., polyethylene glycol (PEG), polypropylene glycol (PPG)), carbohydrate polymer, amino acid polymer or polyvinyl pyrolidone and a fatty acid or fatty acid ester group, each of which can independently comprise from about eight to about seventy carbon atoms. Conjugates for use with a compound of the present technology can also serve as linkers to, for example, any suitable substituents or groups, radiolabels (marker or tags), halogens, proteins, enzymes, polypeptides, other therapeutic agents (for example, a pharmaceutical or drug), nucleosides, dyes, oligonucleotides, lipids, phospholipids and/or liposomes. In one aspect, conjugates can include polyethylene amine (PEI), polyglycine, hybrids of PEI and polyglycine, polyethylene glycol (PEG) or methoxypolyethylene glycol (mPEG). A conjugate can also link a compound of the present technology to, for example, a label (fluorescent or luminescent) or marker (radionuclide, radioisotope and/or isotope) to comprise a probe of the present technology. Conjugates for use with a compound of the present technology can, in one aspect, improve in vivo half-life. Other exemplary conjugates for use with a compound of the present technology as well as applications thereof and related techniques include those generally described by U.S. Pat. No. 5,672,662, which is hereby incorporated by reference herein.

The examples herein are provided to illustrate advantages of the present technology and to further assist a person of ordinary skill in the art with preparing or using the compounds of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### EXAMPLES

### Example 1: Recognition of Single-Stranded Nucleic Acids by Small-Molecule Splicing Modulators

Spinal muscular atrophy (SMA) is one of the most common lethal genetic diseases in new-borns (1, 2). In the most severe type of SMA (type I), infants usually cannot survive beyond their first two years of life due to progressive hypotonia and respiratory failure (3). The cause of SMA in most type I patients is a recessive homozygous deletion within the survival of motor neuron (SMN) 1 gene in chromosome 5 (1, 2). There are two nearly identical SMN genes in humans: SMN1 and SMN2. However, the protein produced by SMN2 cannot fully compensate for the loss of SMN1 in type I SMA patients. Earlier studies demonstrated that a single C-to-T nucleotide (nt) substitution at the +6 position in exon 7 of SMN2 leads to this exon being skipped ˜85% of the time (4), resulting in an inactive SMN isoform. This C-to-T substitution facilitates the preferential binding of a splicing inhibitor, heterogeneous nuclear ribonucleoproteins (hnRNP) A1 (5), over a splicing activator, serine and arginine rich splicing factor 1 (SRSF1)(6). Importantly, the binding of hnRNP A1 shifts the splicing pattern from exon 7 inclusion to a skipped phenotype. With reduced levels of functional SMN protein, the size of motor neurons in the patients' spine is significantly smaller than those in healthy individuals, eventually causing muscle weakness (1).

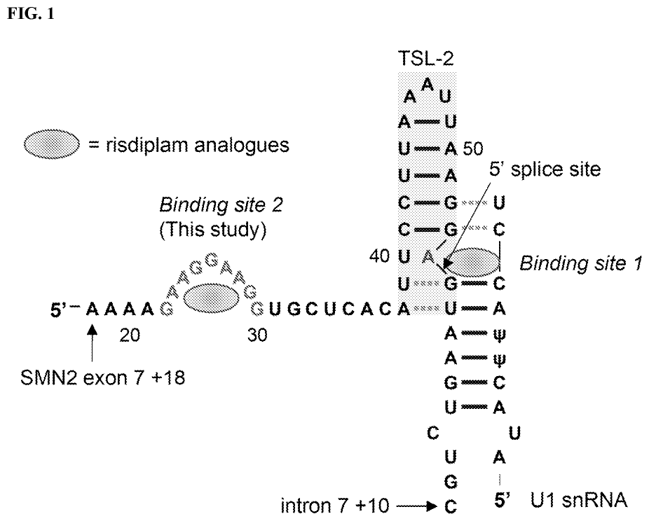

A promising therapeutic strategy is to restore proper splicing of the SMN2 exon 7 to compensate for the loss of the SMN1 gene in SMA patients (2). Following this strategy, there are two existing FDA-approved therapeutics for SMA, namely nusinersen (7) and risdiplam (8). Nusinersen is an antisense oligonucleotide (ASO), which acts by direct binding to the SMN2 pre-mRNA with Watson-Crick base-pairing. The binding site for nusinersen (intron 7+10 to +27 in SMN2) was identified as an intronic splicing silencer (ISS)(9). Blocking this ISS by nusinersen promotes inclusion of exon 7 (10). On the other hand, risdiplam is a first-in-class small-molecule splicing modifier that increases the production of full-length SMN2 mRNA upon oral administration in the SMA mouse model (11, 12) and in humans (13). It was demonstrated that analogues of risdiplam bind directly to exon 7 of the SMN2 pre-mRNA at two separate locations: binding site 1 is located at the 5′ splice site (14, 15) and binding site 2 is a GA-rich sequence located ˜24 nts upstream of the 5′ splice site (FIG. 1). There is a stable splicing-inhibitory RNA element, terminal stem-loop (TSL) 2 between the two putative risdiplam-binding sequences (14, 16). The existence of TSL2 likely makes the two putative binding sites closer in space (FIG. 1).

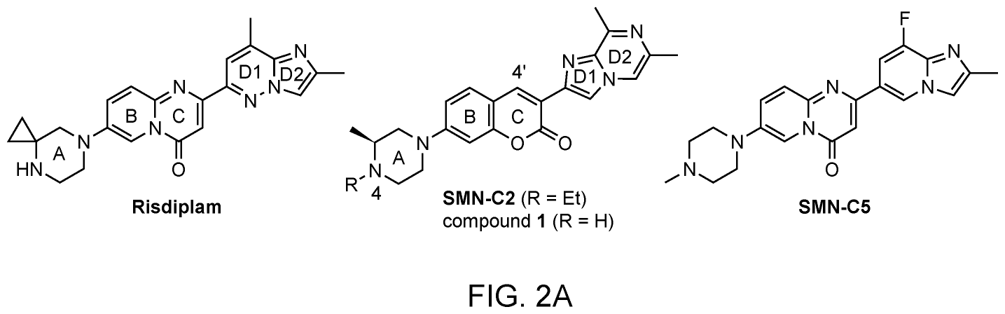

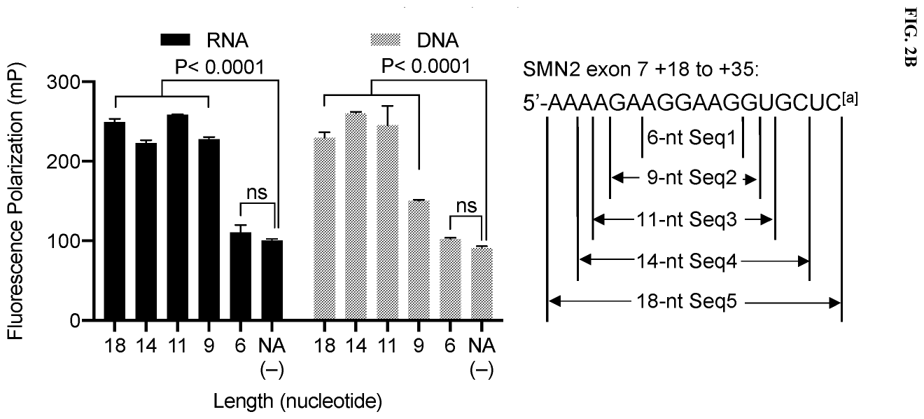

At binding site 1, one of the risdiplam analogues, SMN-C5 (FIG. 2A), was found to stabilize the formation of a ternary complex of the 5′ splice site of the SMN2 exon 7 and the U1 small nuclear ribonucleoprotein (snRNP) via binding to a bulged A (17) (FIG. 1). Without SMN-C5, the 5′ splice site-U1 snRNA duplex is not stable, and the formation of TSL2 is more favourable (17).

At binding site 2, we previously demonstrated that a 15-nt synthetic single-stranded (ss) RNA that contains the GA-rich sequence selectively binds to SMN-C2 (14). Binding site 2 was also identified as part of the exonic splicing enhancer (ESE) 2 (15, 17). The biological consequences for risdiplam analogue binding to binding site 2 include alterations in binding of trans-acting splicing regulatory proteins. Treatment with a risdiplam analogue, SMN-C3 (12), enhanced binding between a 500-nt pre-mRNA sequence containing the GA-rich sequences and a splicing regulatory protein, far upstream element-binding protein 1 (FUBP1) (14). In surface plasmon resonance (SPR) assays, the presence of SMN-C5 prevents the binding of hnRNP G with ESE2 (15). Reverse-genetic studies showed that deletion of the entire ESE2 sequence in a cell-based minigene system reduces the effectiveness of SMN-C5 at restoring proper splicing of SMN2 (15). The effective concentration at 50% potency (EC₅₀) of SMN-C5 significantly increases in ΔESE2 minigene compared to the wildtype sequences, and SMN-C5 failed to induce the complete inclusion of exon7 even at a high concentration (10 μM)(15). However, if only the GA-rich sequence is removed, SMN-C5 can still weakly modulate splicing of the SMN2, indicating that the GA-rich sequence is not strictly required for drug action (15). These findings indicated that the GA-rich sequence plays a role in enhancing the drug potency. The GA-rich sequence is also found in some other risdiplam-sensitive genes (e.g., STRN3 (15, 18)).

In view of this, it was hypothesized that the 5′ splice site is the primary target for the modulatory activity, and that ESE2 facilitates the modulator binding through a cooperative tertiary RNA structure with the 5′ splice site (15). However, because biophysical evidence supporting the existence of such tertiary RNA structure has not been found (14), and because it is not uncommon that small molecules are able to bind to several different sites in large RNAs (19-21), we, therefore, suggest that the two small-molecule binding sites should probably be treated separately rather than as parts of a larger more complicated binding site.

In this study, we interrogated the affinity and selectivity of risdiplam analogues that bind to the GA-rich sequence (binding site 2) in SMN2 exon 7 using a range of biochemical and biophysical tools. Instead of deleting the whole ESE2 sequence, we made single-point mutations within the GA-rich sequence that could disrupt the binding between risdiplam analogues and this GA-rich sequence. Molecular dynamics (MD) has proven useful in simulations of the structural dynamics of nucleic acid (22, 23), notably nucleic acid-ligand interactions (24-27). We performed all-atom simulations using a robust Gaussian accelerated MD (GaMD)(28, 29) method to obtain new insights into the mechanism of risdiplam analogue binding to the target nucleic acids.

### Material and Methods

### Reagents

All synthetic DNA and RNA oligomers were purchased from Integrated DNA Technologies and reconstituted in nuclease-free water (Invitrogen #AM9932) at 1 mM. The concentration of the oligonucleotides at ˜0.1 mM was calibrated with the A260 value measured by a NanoDrop ND-1000 (Thermo, Waltham, MA, USA) and the predicted extinction coefficient (http://www.oligoevaluator.com) using Lamber-Beer's Law. The risdiplam analogues (1 mM in DMSO) were diluted in 2× assay buffer (40 mM HEPES, 200 mM NaCl for RNA; 40 mM HEPES, 200 mM NaCl, 1 mM MgCl₂ for DNA) to make a 1 μM 2× working solution.

### Fluorescence Polarization Binding Assay

A 1:2 dilution series (8 points) of each oligonucleotide was prepared in 20-30 μL water to desired concentrations (i.e., 1-128 μM). 20-30 μL 2× working solution containing the 2× assay buffer and the small-molecule ligand was added to each oligonucleotide sample in 1:1 (v/v) and mixed by pipetting for 4 times. For fluorescence polarization measurement, 20 μL of the above 1× working solution was transferred into a 384-well, black, F-bottom microplates (Greiner Bio-One) in duplicates or triplicates. The plate was equilibrated at room temperature for 5 min before being read using a microplate reader (SYNERGY H1, BioTek; Excitation/Emission=360/460 nm) at 25° C. For compound SMN-C5, a competitive binding assay was employed to measure an apparent K_{d}. SMN-C5 was 1:2 serial diluted for 10 concentration points starting at 750 μM in DMSO. A mixture of SMN-C2 (0.5 μM) and DNA Seq6 (25 μM) in 1× assay buffer was prepared and incubated at room temperature for 10 minutes. 3 μL of SMN-C5 solution at each concentration was added to a 42 μL SMN-C2/DNA Seq6 mixture and incubated at room temperature for 10 minutes. The mixture was then transferred into a microplate (20 μL each well) in duplicates for fluorescence polarization readout. The experimental data were analyzed using Prism 8 software (Graphpad Software, San Diego, CA, USA). The dissociation constant (K_{d}) was calculated with 95% confidence interval after nonlinear curve fitting (Sigmoidal, 4 parameters).

### Gaussian Accelerated Molecular Dynamics (GaMD) Simulations

GaMD works by adding a harmonic boost potential to smooth the potential energy surface and reduce system energy barriers. GaMD provides unconstrained enhanced sampling without the requirement of pre-defined collective variables. Moreover, because the boost potential exhibits a Gaussian distribution, biomolecular free energy profiles can be properly recovered through cumulant expansion to the second order (29). GaMD has been demonstrated to accelerate biomolecular simulations by orders of magnitude, especially for ligand binding to proteins (30, 31). Here, we have applied GaMD to explore the binding of the risdiplam analogue to the putative target nucleic acids with GA-rich sequence. The simulation structures of nucleic acids were built using NAB in the AmberTools package (32). The nucleotide sequence was used to construct the Arnott B-Right handed DNA and RNA duplexes. One of the strands from each duplex was extracted to generate the starting nucleic acid structure (Seq6) of DNA and RNA. A ligand molecule of compound 1 was placed randomly at >15 Å away from the nucleic acid. Each simulation system was then prepared using the solution builder plug-in with the CHARIMM-GUI web server (33). Each system was solvated in 0.1M NaCl solution at 298.15 K. The AMBER force field, BSC1 was used for DNA, OL3 for the RNA, GAFF2 for ligand and TIP3P for water in the system.

Initial energy minimization, equilibration, and conventional molecular dynamics (cMD) of compound 1 binding to the DNA and RNA Seq6 were performed using the output files from CHARMM-GUI. Specifically, the system was energy minimized using the steepest descent for 2500 steps and conjugate gradient for another 2500 steps. After minimization, the system was heated from 0 to 298.15 K in 125 ps simulation by applying 1.0 kcal/(mol·Å²) harmonic position restraints to DNA/RNA and ligand heavy atoms with a constant number, volume and temperature (NVT) ensemble. The system was further equilibrated for 125 ps at 298.15 K using constant number, pressure and temperature (NPT) ensemble with the same restraints as in the NVT run. Then cMD without any constrains was performed to further relax the system for 1 ns at 1 atm pressure and 298.15 K temperature.

The simulations proceeded with GaMD (see method details in SI), which was implemented in the GPU version of AMBER18 (34). A short cMD run of 2 ns was performed to collect potential statistics (including the maximum, minimum, average and standard deviation (SD)). Then 40 ns GaMD equilibration after applying the boost potential was performed. All GaMD simulations were run at the “dual-boost” level by setting the reference energy to the lower bound; i.e., E=Vₘₐₓ (28, 29). One boost potential is applied to the dihedral energetic term and another to the total potential energetic term. The average and SD of the system potential energies were calculated every 200,000 steps (400 ps) for both systems. The upper limit of the boost potential SD, σ0 was set to 6.0 kcal/mol for both the dihedral and the total potential energetic terms for DNA-ligand binding system. While the σ0 was set to 3.0 kcal/mol and 6.0 kcal/mol for the total and dihedral potential energetic terms in the RNA-ligand binding system, respectively. Finally, five independent 500 ns dual-boost GaMD production simulation runs were conducted with randomized initial atomic velocities. The boost potential was applied to the total and dihedral potential energies of the system. The GaMD production simulations are summarized in Table 5. In all simulations, the hydrogen-heavy atom bonds were constrained using the SHAKE algorithm and the simulation time step was set to 2.0 fs. The particle mesh Ewald (PME) method was employed to compute the long-range electrostatic interactions and a cutoff value of 9.0 Å was applied to treat the non-bonded atomic interactions. The temperature was controlled using the Langevin thermostat with a collision frequency of 1.0 ps⁻¹.

Trajectory analysis was carried out using VMD (35) and CPPTRAJ (36). Hierarchical Agglomerative clustering in CPPTRAJ was performed on the whole system consisting of both the ssDNA/ssRNA and ligand. Representative conformations of each system were identified from the top 10 structural clusters. The centre-of-mass distance between the ligand and DNA and the DNA radius of gyration (Rg) were calculated using CPPTRAJ. They were also used as reaction coordinates for calculating a 2D PMF free energy profile by reweighting all five GaMD simulations combined (see method details for energetic reweighting of GaMD simulations in SI). The PyReweighting toolkit (37) was used for reweighting the GaMD simulations. A bin size of 1 Å was used for distance and Rg and cutoff set to 500 frames in a bin or cluster for reweighting.

### NMR Studies

NMR spectra were acquired on a Bruker 600 MHz Avance III HD spectrometer equipped with a TCI cryoprobe, and a Varian INOVA 600 MHz spectrometer equipped with a cryogenic HCN probe. To a 1 mL Eppendorf tube was added 4 μL or 40 μL (for 10 μM or 100 μM final concentration) 500 μM compound 1 solution, followed by 20 μL 10×PBS pH 5.0 buffer, 10 μL D₂O, 2 μL DMSO-d6 and H₂O. The solution was then heated at 60° C. for 1 min before 1 mM DNA Seq4 solution was added to form a 200 μL small molecule-DNA solution. The amount of the DNA Seq4 solution and H₂O were varied to form solutions of different ratios of DNA Seq4: compound 1. The series of solutions with 2.5 mol %, 5 mol %, 10 mol %, 20 mol %, 50 mol %, 100 mol %, 200 mol %, 400 mol %, 1000 mol %, 2000 mol %, 5000 mol % and 8200 mol % DNA Seq4 were then transferred to 3 mm NMR tubes for analysis. The ¹H NMR spectra were acquired at 40° C. with wet1D or excitation-sculpture (ES) to suppress the water signals. The STD experiment was performed with selective saturation at 5.80 ppm with a selective bandwidth of 125 Hz, 2 s mixing time, 16 k scans.

We previously reported that a risdiplam analogue, SMN-C2 (FIG. 2A), binds to a 15-nt GA-rich sequence in SMN2 exon 7 (14). SMN-C2 was observed to bind to GA-rich DNAs similarly to RNAs (FIG. 2B). Because DNA oligonucleotides are more readily accessible than RNA (the unit price for synthetic RNA is more than 10 times of DNA), we extensively used synthetic DNA sequences to probe small-molecule-nucleic acid interactions throughout this study, and RNA sequences were used to validate important findings. Here, we first investigated the minimum length of sequence required for SMN-C2 binding. Fortunately, the fluorescent coumarin moiety of SMN-C2 (ring B/C) permits the use of a fluorescence polarization (FP) assay to interrogate said binding. Applying different-length RNAs and DNAs at 50 μM, we found that a 9-nt sequence (i.e., GAAGGAAGG) is the minimum length required for SMN-C2 binding (FIG. 2B). To explore the tolerable and preferential binding sequences for SMN-C2 binding, we added a single overhang nucleotide at both the 5′ and the 3′ ends of a cohort of 9-nt RNA/DNA sequences (Table 1), because the overhang nucleotides may provide extra stability to the nucleic acid-SMN-C2 complex (Table 6). The U/T was chosen as the overhang nucleotide because we previously reported that SMN-C2 has the least binding affinity to the pyrimidines (14). Replacing either end of the 9-nt core sequence with one or two “inactive” Us in RNA (or Ts in DNA), significantly weakened the SMN-C2-binding affinities (Table 1, Seq6 vs Seq7-9), thus validating that the 9-nt GA-rich sequence is the minimum length required for SMN-C2 binding.

TABLE 1
Binding affinitiesª of SMN-C2 and
ssDNAs^{b} or ssRNAs, which harbour
a 9-nucleotide GA-rich sequence.

| | | RNA | DNA |
|---|---|---|---|
| Seq | Sequence^{b,e} | K_{d} (μM) | K_{d} (μM) |
| 6^{b} | UGAAGGAAGGU | 17.5 ± 2.2 | 3.1 ± 0.7 |
| 7^{b} | UUAAGGAAGGU | 42.3 ± 13.3 | >100 |
| 8^{b} | UGAAGGAAGUU | >100 | 14.7 ± 0.7 |
| 9^{c} | TTAAGGAAGTT | N.T. | >100 |
| 10^{b} | UAAAGGAAGGU | >100 | >100 |
| 11^{c} | TGGAGGAAGGT | N.T. | 57.3 ± 20.0 |
| 12^{b} | TGAAAGAAGGT | N.T. | 17.6 ± 3.4 |
| 13^{b} | UGAAGAAAGGU | >100 | >100 |
| 14^{c} | AGAAGGTAGGT | N.T. | >100 |
| 15^{c} | TGAAGGATGGT | N.T. | 41.1 ± 4.4 |
| 16^{c} | TGAAGGAAAGT | N.T. | 27.4 ± 6.0 |
| 17^{c} | TGAAGGAAGAT | N.T. | 56.4 ± 10.4 |
| 18^{d} | Binding | 60 ± 36 | N.T. |
| | site 1 | | |
| | sequence | | |

^{a}Determined by the fluorescence polarization assay with 0.5 μM SMN-C2. The K_{d} range is the 95% confidence interval of the calculated 50% response concentration from the Sigmoidal interpolation. All dose titrations were reproduced in three replicates.
^{b}In DNA sequences, deoxyribonucleotides, dA, dT, dG, and dC, were used in substitution of A, U, G, and C for RNA.
N.T. = not tested.
^{c}Only DNA sequences were investigated.
^{d}For comparison, a 1:1 annealed mixture of the U1 snRNA binding sequence, 5′-AUACΨΨACCU (Ψ = pseudouridine), and the 5′ splice site, 5′-GGAGGUAAGUCU to resemble binding site 1 (17).
^{e}The underlined positions indicate the single-point nucleotide differences from the 9-nt putative binding sequence.

Next, we mutated the 9-nt core nucleotides to investigate the sequence specificity of SMN-C2 binding. Importantly, any single-point mutation of the core sequence as shown in Table 1 resulted in at least a 5-fold decrease in binding affinity (Seq10-17) even by merely replacing some single G into A or vice versa, highlighting the specificity of SMN-C2 binding (Table 1, Seq10-13, 16, 17). After investigating the binding of SMN-C2 with an additional 25 sequences in the context of 18-nt DNA, we identified the complete consensus sequence for SMN-C2 as GARGGARGG (R=A/G) for DNA (Table 7). Double-stranded (ds) RNA Seg18 represents the duplex formed between the 5′ splice site of SMN2 exon 7 and U1 snRNA, and was previously used in NMR studies for binding site 1 (17). Comparing the two risdiplam putative binding sites side-by-side, the binding affinity (K_{d}) of SMN-C2-RNA Seq6 (binding site 2) is 3.4-fold stronger than that observed with RNA Seg18 (Table 1). The binding affinity of SMN-C2-dsRNA Seg18 determined by the FP assay (K_{d}=60±36, Table 1) is generally consistent with the literature, where the affinity of this dsRNA to another risdiplam analogue, SMN-C5, was determined as K_{d}=28±9 μM in an NMR chemical shift assay (17).

### Mutations in the GA-Rich Sequence Reduced the Drug Effect in Cells

To evaluate if the GA-rich sequence facilitates the activity of risdiplam analogues, two single-point mutations from Table 1 were individually introduced in the cell-based minigene system (modified from pCI-SMN2 plasmid (4), see Supporting Information): (i) exon 7+22G>T (M1, mutation in Seq7) and (ii)+22G>A (M2, mutation in Seq10). In 293T cells that were transfected with the mutated minigenes, SMN-C2 can still significantly rescue exon 7 inclusion (>90%) at 1 μM (FIG. S6). However, the EC₅₀ was ˜2-3-fold higher than that observed with the minigene with a wildtype exon 7 sequence, indicating that GA-rich sequence is relevant to the mechanism of SMN-C2 and is pivotal to maintaining the potency of SMN-C2. Compared to the previous reverse genetic studies that deleted the whole ΔESE2 sequence (15), these results demonstrated that even a single-point mutation in the GA-rich sequence is sufficient to lessen the drug effect. This is consistent with the hypothesis that the GA-rich sequence serves as a secondary RNA target that facilitates SMN2 exon 7 splicing (17).

SMN-C2 and ssDNA Formed a 1:1 Complex

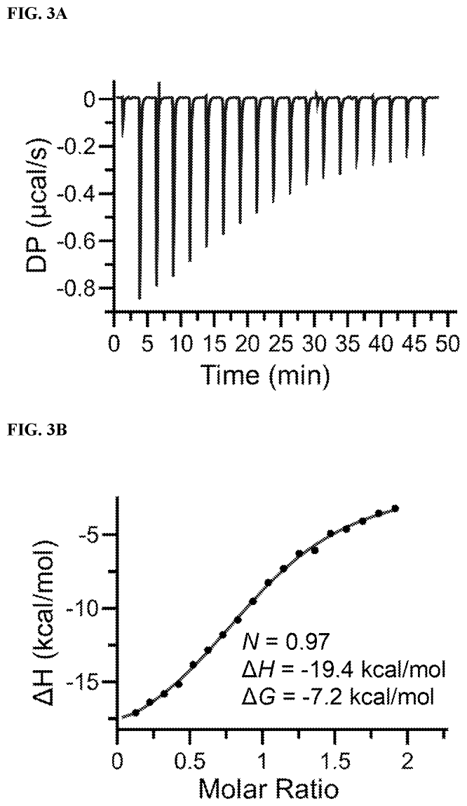

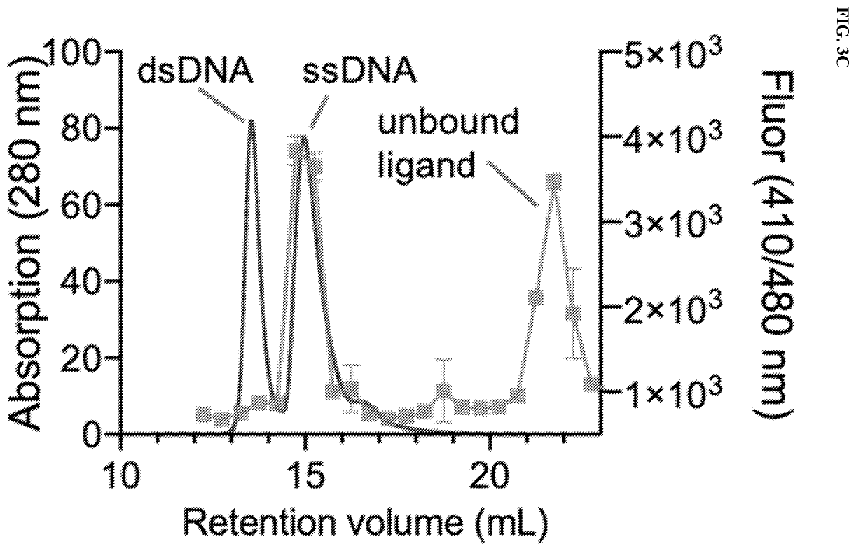

We first measured the stoichiometry and reconfirmed the binding affinity of DNA Seq6 and SMN-C2 by isothermal calorimetry (ITC). They showed a 1:1 binding stoichiometry with a calculated K_{d} of 4.8 μM (FIGS. 3A and 3B), which is close to the K_{d} observed with the FP assay (Table 1, DNA Seq6). Interestingly, a dsDNA formed by annealing Seq6 and its reverse complement, Seq6_RC, abrogated most of the binding affinity (FIG. S7). In the same experimental settings, RNA Seq6 generated significant solvation heat, which made us unable to obtain an accurate ITC measurement. Second, the possibility of DNA oligomerization (e.g., G-quadruplex formation) was ruled out by size-exclusion chromatography. The retention volume for the 14-nt DNA (Seq4) was consistent with its reverse complement (Seq4_RC) and was well resolved from the annealed dsDNA (Seq4+Seq4_RC), in which the molecular weight almost doubles (FIG. S8). In addition, SMN-C2 did not significantly change the retention volume of the DNA Seq4 (FIG. S8). The results suggested that although DNA Seq4 contains six Gs, it cannot form a G-quadruplex at 100 μM in the presence or absence of SMN-C2. An 11-nt DNA sequence that contains two GGG segments (Seq19=TGAGGGAGGGT) can however form an intermolecular G-quadruplex at 100 μM as the retention volume in size-exclusion chromatography was similar to that observed with a random 45-nt ssDNA (FIG. S9). Importantly, in the presence of dsDNA Seq4 (annealed Seq4 and Seq4_RC), the elution of SMN-C2 was concurrent with the ssDNA but not the dsDNA peak (FIG. 3C), reconfirming that SMN-C2 preferentially binds to the ssDNA over the dsDNA Seq4. Collectively, our results suggested that SMN-C2 binds to the monomeric single-stranded GA-rich sequences in a 1:1 ratio.

### GaMD Simulations Suggested a Mechanism for Small-Molecule-Nucleic Acid Binding

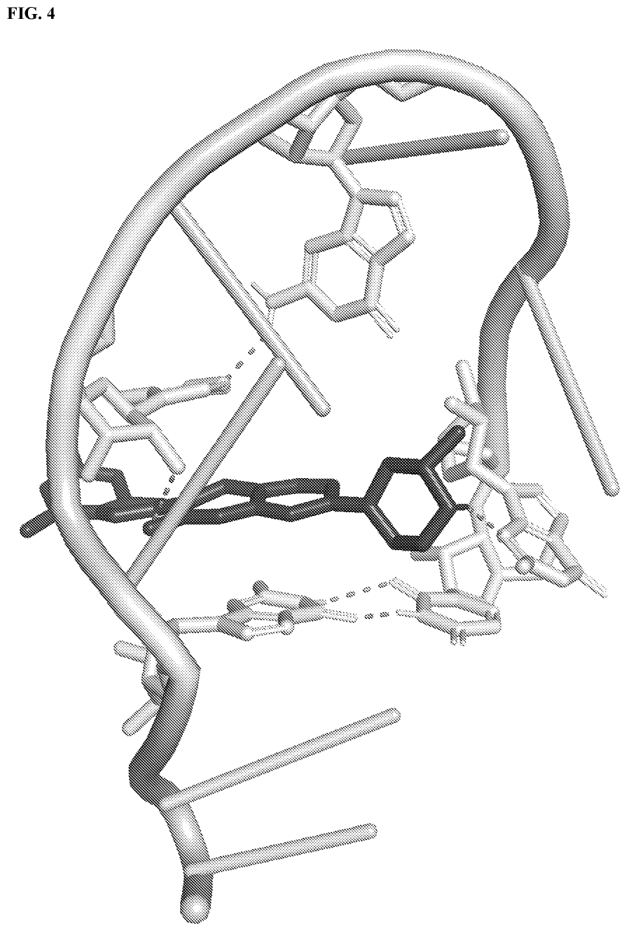

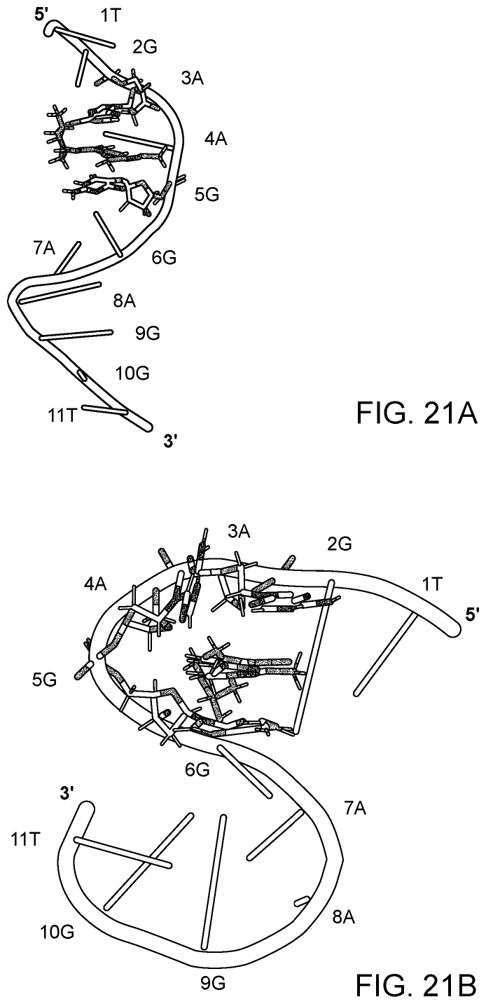

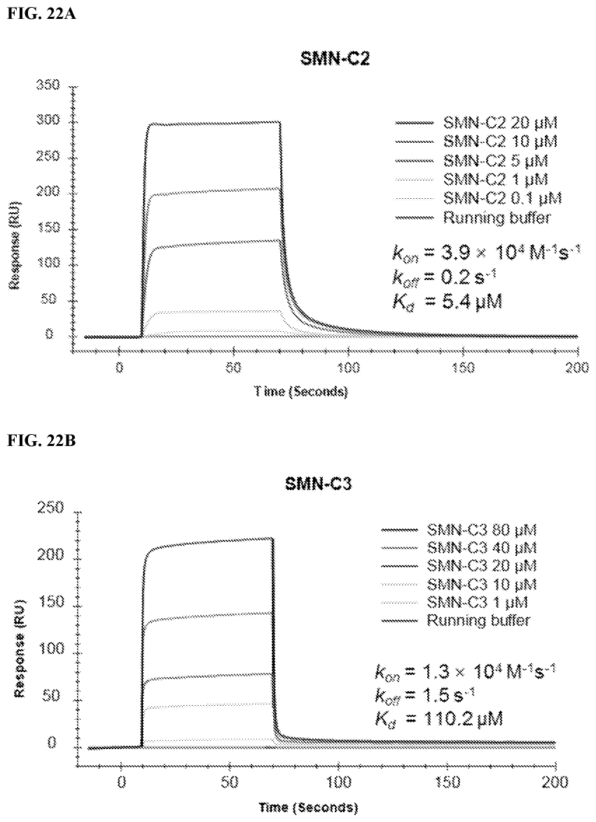

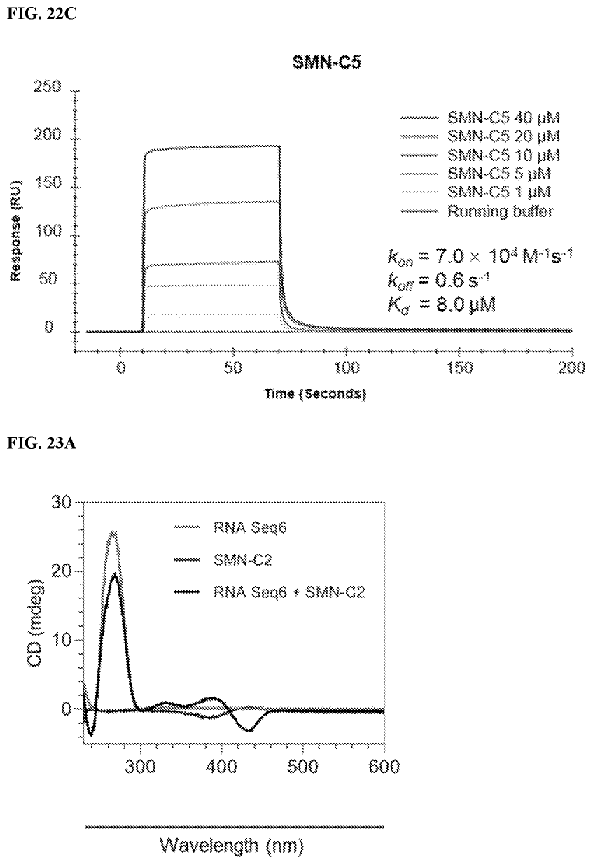

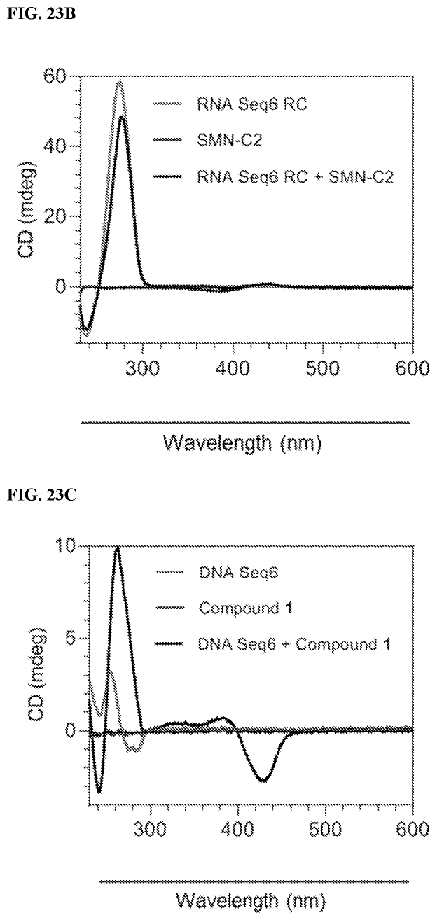

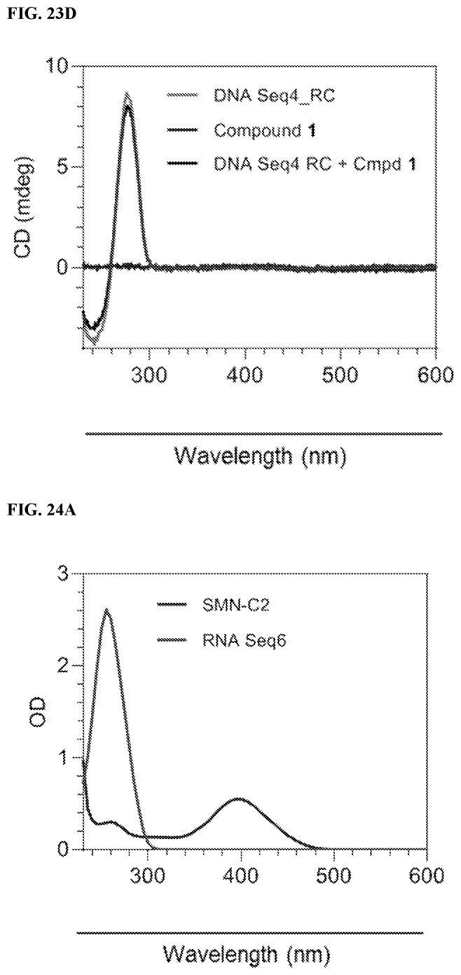

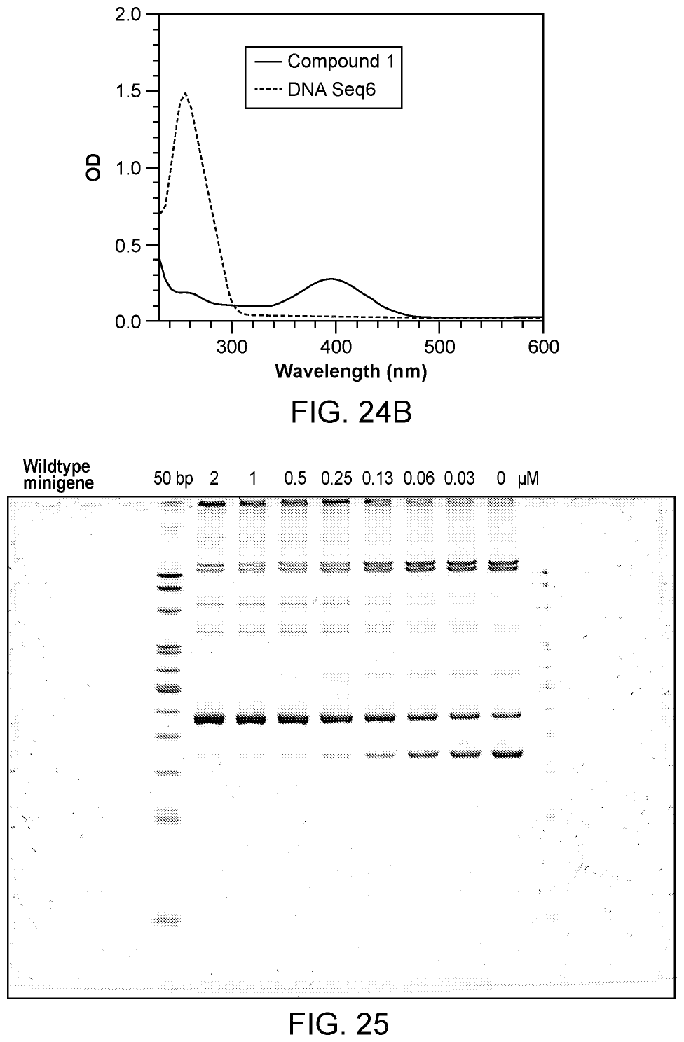

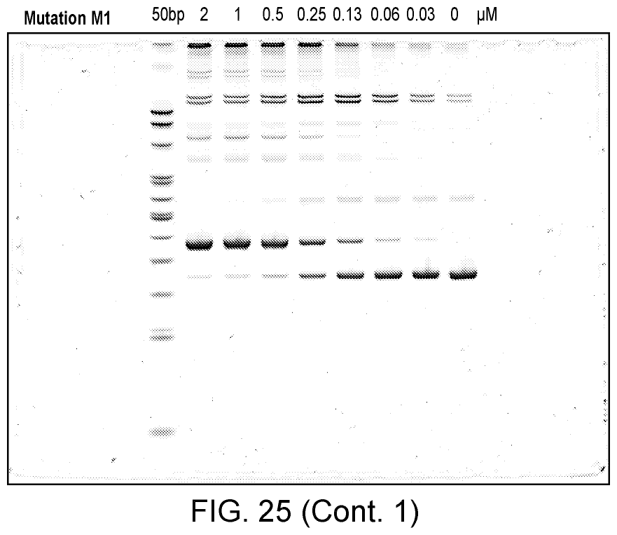

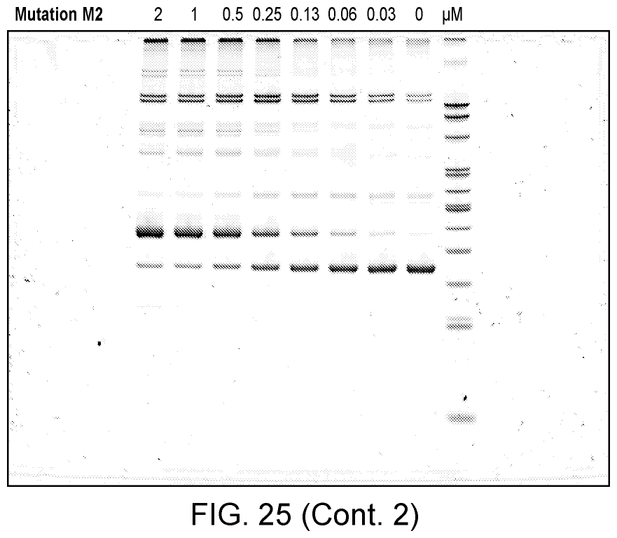

All-atom GaMD simulations were used to study the molecular interactions responsible for the binding of compound 1 (FIG. 2A) to the putative nucleic acid target sequence. DNA and RNA Seq6 were chosen because they represent one of the best nucleic acid binding receptors for risdiplam analogues (Table 1). Compound 1, a more water-soluble analogue of SMN-C2, is used in simulations for being consistent with the NMR experiments (see below). During five independent 500 ns GaMD production simulations, compound 1 appeared to spontaneously bind to both DNA and RNA Seq6. When bound to DNA Seq6, the centre-of-mass distance between DNA and ligand reduced to ˜4.5 Å (FIG. 4A) and DNA radius of gyration (R_{g}) reduced to ˜8.0 Å (FIG. 4B). A 2D free energy profile was then calculated through reweighting of the five GaMD simulations combined. Three low-energy conformational states of the system were identified (FIG. 4C), for which structural clustering was performed to obtain the representative system conformations, including the “Bound/Unfolded” (FIG. 21A), “Bound/Intermediate” (FIG. 21B) and “Bound/Folded” states. Compound 1 was able to interact with DNA Seq6 in all three states. In the Bound/Folded state, compound 1 bound to folded DNA, forming a compact structure (FIG. 4). Two successive GAAG loop-like structures (38, 39) were identified in the folded DNA for binding of the compound 1.

Three low-energy conformational states of the RNA-ligand system were also identified for RNA Seq6, including the “Unbound/Unfolded” (FIG. S20b), “Intermediate” (FIG. S10A) and “Bound/Folded” states (FIG. 4). In the Bound/Folded state, a similar binding mode of compound 1 was observed in RNA as in the DNA with subtle differences. In DNA, the coumarin core (ring B/C) of compound 1 intercalated between the 2^{nd} and 4^{th} bases of the first GAAG motif (FIG. 4), whereas in RNA, the intercalation occurred between the 2^{nd} and 4^{th} bases of the second GAAG motif. In both DNA and RNA, it appears that the ARG trinucleotide in the GAAG motif is thus important for binding of compound 1 through π-stacking interactions. Even though both DNA and RNA Seq6 contained two GAAG motifs, the nucleic acids formed compact sequential loop-like structures (FIG. 4), which could accommodate only one small molecule. In the simulations for RNA Seq6, the unfolded states appeared to be less stable than the unfolded states in DNA, and compound 1 did not spontaneously bind to this unfolded state (FIG. S20B).

NMR Studies of Compound 1 Binding to ssDNA

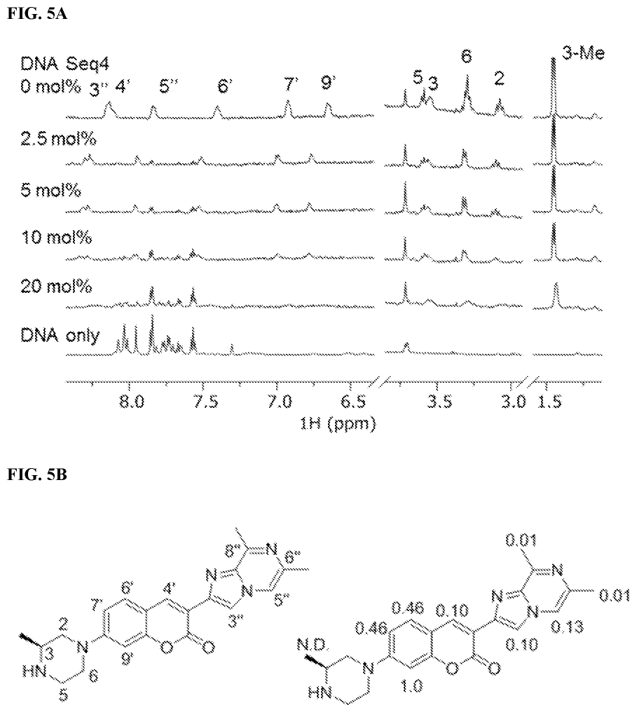

We performed a series of NMR experiments to validate the simulated binding modes of compound 1 to the target nucleic acid. First, DNA Seq4 was titrated into a solution containing 100 μM compound 1 and the ¹H NMR spectra were measured. The peaks from compound 1 shift upon the addition of 2.5 mol % and broaden as the concentration of DNA increases to 20 mol % (FIG. 5A). The line-broadening effect is similar to the reported observation for the binding of SMN-C5 and the dsRNA of the 5′-splice site of SMN2 exon 7 and U1 snRNA (Seq18)(17). Specifically, aromatic signals of compound 1 (6.6-8.5 ppm) are below the detection limit when 20 mol % of the DNA Seq4 is present, whereas the aliphatic signals are still observed (1.3-4.1 ppm, FIG. 5A). The peak width at half maximum plus the J coupling constant (FWHM+J) of the doublet for 3-CH₃ of compound 1 (˜1.4 ppm) only increases from 10.4 Hz at 0 mol % DNA to 13.5 Hz at 20 mol % DNA (FIG. 5A). The reduced attenuation of the aliphatic signals relative to aromatic signals upon addition of DNA Seq4 suggested that the piperazine ring (ring A) retains dynamics similar to the free ligand, whereas the rings B/C and D1/D2 exhibit behavior associated with nucleic acid binding. The NMR titration also suggested that a heterogeneous binding conformation exists, because there were no bound-form compound 1 peak reappearing even if an 82-fold excess DNA Seq4 was added.

Next, saturation transfer difference (STD) experiments were carried out to further interrogate the interaction between compound 1 and DNA Seq4 (FIG. 5B)(40). The anomeric proton resonances of the deoxyriboses in DNA Seq4 were selectively saturated at 5.80 ppm with a bandwidth of 125 Hz, and STD signals of compound 1 were measured (FIGS. 5A and 5B/b). The aromatic rings B/C and D1/D2 in compound 1 demonstrated strong STD, while the piperazine moiety yielded no observable STD peaks. The 6″- and 8″-methyl groups (ring D2) also showed small STD signals compared to those observed with the aromatic protons. Thus, both the ¹H NMR titration and the STD experiments strongly indicated that the interaction between compound 1 and the DNA Seq4 is mainly driven by the aromatic moieties, specifically rings B, C, and D1, of this small molecule. This result is also consistent with the fact that SMN-C2 prefers to bind with purine-rich sequences since the latter engage in 71-interactions more effectively than pyrimidine-rich sequences (41).

### Structure-Affinity-Relationship Studies of Risdiplam Analogues

To further probe the nucleic acid interactions with risdiplam analogues, we set out to alter the small-molecule structures. Specifically, we compared two known active risdiplam analogues, SMN-C2 and -C5 (FIG. 2A)(11), and synthesized additional 11 risdiplam analogues containing the coumarin core (Scheme 1). In this cohort of compounds, only SMN-C5 contains a pyridopyrimidinone core (ring B/C), instead of a coumarin core, which is not fluorescent. Utilizing competitive FP and surface plasmon resonance (SPR) assays, we observed that SMN-C5 demonstrated similar binding affinities to those observed with SMN-C2 (Table 2, entries 2 and 3). In the newly synthesized collection of risdiplam analogues (Table 2, Scheme 1), either removing the substituents on the N4 position of the piperazine ring (compound 1, ring A) or extending N4 with a bulky butyloxycarbonyl (Boc) group (compound 2) does not substantially alter the binding affinity with DNA Seq6 (Table 2). This is consistent with the NMR studies which showed that the piperidine portion of the molecule does not interact with DNA (FIG. 5). Interestingly, the Boc group reduced the binding of compound 2 to RNA Seq6 by ˜3 fold, suggesting that ligand binding mode between DNA and RNA Seq6 may have some differences (Table 1, RNA Seq6 vs Table 2, entry 6).

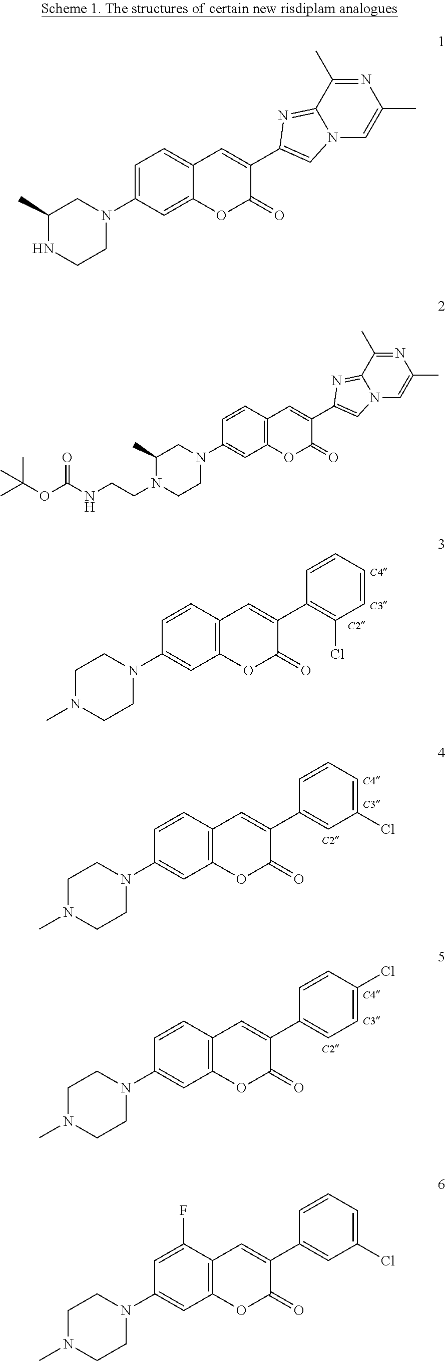

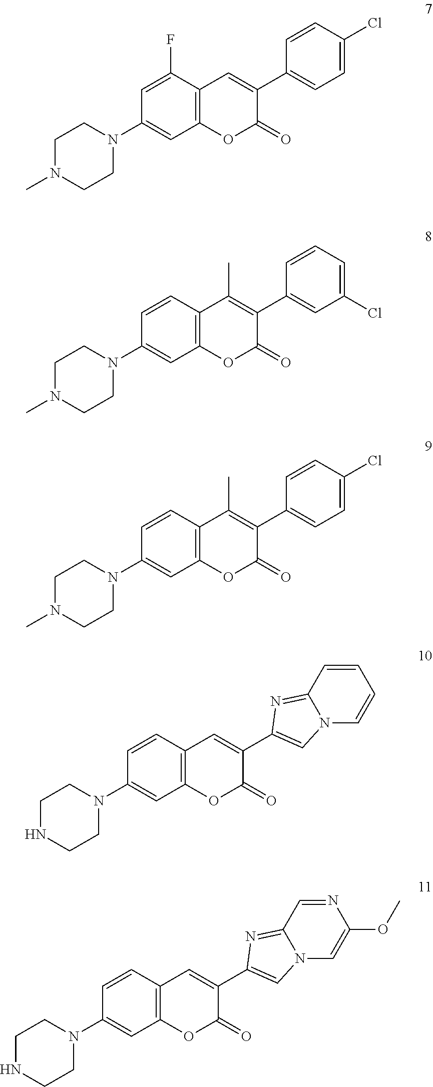 [SMILES: CN1CCN(c2cc(F)c3cc(-c4ccc(Cl)cc4)c(=O)oc3c2)CC1 ; Cc1c(-c2cccc(Cl)c2)c(=O)oc2cc(N3CCN(C)CC3)ccc12 ; Cc1c(-c2ccc(Cl)cc2)c(=O)oc2cc(N3CCN(C)CC3)ccc12 ; O=c1oc2cc(N3CCNCC3)ccc2cc1-c1cn2ccccc2n1 ; COc1cn2cc(-c3cc4ccc(N5CCNCC5)cc4oc3=O)nc2cn1]

TABLE 2
Binding affinities of the risdiplam
analogues^{a} and the GA-rich DNA and/or RNA sequences.

| | | | SMN2 EC₅₀ |
|---|---|---|---|
| # | Compounds | K_{d} (μM) | (μM)^{e} |
| 1 | SMN-C2 | 5.4^{b} | 0.26 ± 0.13 |
| 2 | SMN-C5 | 8.0^{b} | 0.059 ± 0.009 |
| 3 | SMN-C5 | 5.8 ± 2.5^{c} | |
| 4 | compound 1 | 2.9 ± 0.3^{c} | 0.014 ± 0.003 |
| 5 | compound 2 | 4.0 ± 0.3^{c} | |
| 6 | compound 2 | 51.2 ± 4.9^{d} | >8 |
| 7 | compound 3 | >100^{c} | >8 |
| 8 | compound 4 | 13.9 ± 1.1^{c} | >8 |
| 9 | compound 5 | 8.2 ± 0.7^{c} | >8 |
| 10 | compound 6 | 18.7 ± 4.0^{c} | >8 |
| 11 | compound 7 | 9.9 ± 0.9^{c} | >8 |
| 12 | compound 8 | >100^{c} | >8 |
| 13 | compound 9 | >100^{c} | >8 |
| 14 | compound 10 | 2.8 ± 0.5^{c} | >8 |
| 15 | compound 11 | 1.4 ± 0.2^{c} | >8 |

^{a}For structures of entries 4-15, see Scheme 1. For FP experiments, the K_{d} range is the 95% confidence interval of the calculated 50% response concentration from the Sigmoidal interpolation. All dose titrations were reproduced in three replicates. The K_{d} values were measured using the
^{b}SPR assay for RNA Seq4,
^{c}FP assay for DNA Seq6, or
^{d}FP assay for RNA Seq6.
^{e}The EC₅₀ of the cell-based SMN2 splicing assay in 293T cells (56).

We also truncated the bicyclic ring D1/D2 (see FIG. 2A for assignment) in SMN-C2 into monocyclic structures with various substituents. The position of the Cl substituent on the monocyclic ring D is crucial for the binding affinity (compounds 3-5). Changing the Cl position from C4″ into C2″ reduces the binding affinity by more than 12-fold (compounds 3-5, Table 2), probably because the 2″-Cl forces ring D1 out of coplanarity. In general, the analogues with a monocyclic ring D have weaker binding affinities to DNA Seq6 than that observed with SMN-C2 (Table 1, DNA Seq6 vs Table 2, compounds 3-9). Unlike these compounds with a monocyclic ring D, the new risdiplam analogues with a bicyclic ring D1/D2 (compounds 10 and 11) both showed high binding affinity to the GA-rich sequence DNA Seq6 when the ring D1 is unchanged from SMN-C2 (compounds 10 and 11, Table 2).

### The Secondary Structures Enhanced the Loop-Like Conformation and SMN-C2 Binding

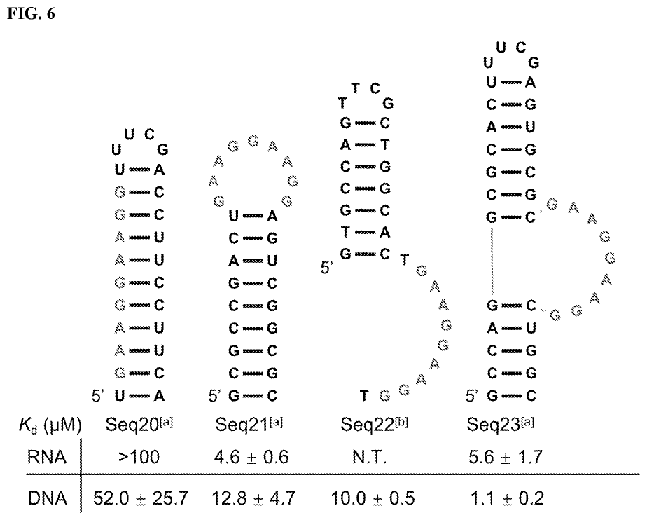

To further validate the double loop-like conformation in SMN-C2 binding, we synthesized several oligonucleotides that can stabilize or destabilize the folded conformation of the core sequence by base-pairing (FIG. 6). As expected, when additional nucleotides complementary to the core sequence are appended to the 3′-end (DNA and RNA Seq20), the putative binding sequence in the stem region cannot form the double loop-like conformation and, therefore, Seq20 has the lowest binding affinity for SMN-C2. On the contrary, the binding affinity is much higher when the core sequence is contained within a single-stranded RNA loop (Seq21) or DNA bulge (Seq23). The conformationally constrained sequences arising from the complementary base-pairing interactions at the ends of the GA-rich sequences in RNA Seq21 and DNA Seq23 likely stabilize the double loop-like structures, resulting in more favourable ligand binding. These results further support the simulation models where the distance between the 5′ and 3′ ends of the GA-rich sequences is quite short in the Bound/Folded states (FIG. 4).

### DISCUSSION

We previously demonstrated that the GA-rich sequence in exon 7 is duplexed with the 3′-end of intron 6 and forms a bulged stem-loop structure (TSL1) in vitro and in cells (14). The binding of SMN-C2 only makes subtle changes in the conformation judged by selective 2′-hydroxyl acylation analysed by primer extension (SHAPE) experiments (14). However, the TSL1 structure must be linearized to ssRNA to be recognizable by some splicing regulatory proteins, such as hnRNP A1 (5) and Tra2β1 (42). The results in this report show unambiguously that SMN-C2 binds to the single-stranded GA-rich sequence, indicating that the single-stranded conformation in this GA-rich region is functionally relevant for both the potency of SMN-C2 and trans-acting regulatory proteins.

Unlike antisense oligonucleotides that bind to specific RNA sequences, small molecules usually recognize RNA secondary or tertiary structures (43-45). These structures can be simple internal bulges that contain 2-6 unpaired nucleotides (e.g., ref (46)) or complex riboswitches that contain a small-molecule binding cavity, which cannot be discerned from primary sequences (e.g., ref (47)). In the past few years, RNA-small molecule interaction databases have been built based on RNA structural patterns, including Inforna (48, 49) and R-BIND (50). It is difficult, however, to discern likely secondary structures within the short 9-nt primary GA-rich sequence. Nevertheless, the simulation results predicted a plausible and novel aptamer conformation, i.e., a double loop-like structure. GNRA (R=A or G, N=A, U, G, or C) is a common tetraloop turning sequence in RNA stem-loops (51). GAAG tetraloop is a naturally occurring variation of the GNRA tetraloop (38, 52). In single GAAG tetraloops, a closing base pair (e.g., G-C) is often located at the 5′ and 3′ ends to conformationally constrain and thus stabilize the tetraloop structure (38, 51). In the 9-nt GA-rich sequence that consists of two loop-like structures, the compact structure is probably stabilized by the intercalation of compound 1. As an analogy of GNRA tetraloop in RNA, GNNA and GNAB (B=C, G, or T) tetraloops can also stably form in DNA (53), which is consistent with the simulation findings that the double GAAG loop-like structures formed similarly in both DNA and RNA in the presence of compound 1. Constraining the ends of the GA-rich sequence using complementary base-pairs (Seq21 and Seq23, FIG. 6) strengthened the small-molecule binding, generally supporting the Bound/Folded simulation states where the distance between the ends of the GA-rich sequence was predicted to be short (FIG. 4).

The MD simulation results are highly consistent with the NMR and structure-affinity-relationship studies. In the NMR titration experiment (FIG. 5A), aromatic NMR peaks in compound 1 almost all disappeared when the DNA Seq4 concentration reached 20 mol %, suggesting a fast equilibrium among heterogenous binding states. This is consistent with the free energy surfaces arising from the GaMD simulations, which depict at least three ligand-bound states separated by relatively shallow barriers (FIG. 4C). Compound 1 formed a key π-π stacking interaction between ring D1 and the 2nd and 4th nucleobases in the 5′ GAAG loop-like structure in DNA Seq6 (3A and 5G in Seq6). Since the dipole moments of aromatic rings are important for π-stacking (54), this binding model was consistent with experimental findings that the ligand-binding was sensitive to the A-to-G or G-to-A substitutions in 3A and 5G (Table 1, Seq11 and 12). The simulated binding mode is also consistent with the relatively strong NMR STD in ring B/C of compound 1, which, in simulations, formed π-π and lone pair-π stacking interactions with 5G and 3A in DNA Seq6, respectively (FIGS. 4 and 5B). In addition, simulations demonstrated that the 3-methyl in the piperazine ring (ring A) and 6″- and 8″-methyl groups in ring D2 were solvent accessible, consistent with the low STD in NMR in both rings (FIGS. 4 and 5B).

In RNA Seq6, simulation revealed that the piperazine ring in the compound interacted with the RNA aptamer via a polar bond between N4 of compound 1 and N3 of the 2G nucleobase (FIG. 4). This is consistent with the finding that the GA-rich RNA was more sensitive to N4 alkylation in the SMN-C2 scaffold than that observed with DNA. A bulky Boc group reduced the binding affinity for RNA Seq6 by 3-fold (Table 1, RNA Seq6 vs Table 2, entry 6). It was also shown in simulation results that the double loop-like ligand-binding pocket was confined by 2G and 5G in DNA Seq6 (FIG. 4). Methyl substitution at C4′ of the coumarin (compounds 8-9, Table 2) likely resulted in a steric clash with the 5G nucleobase and therefore the binding affinity was reduced, while a smaller F group at C6′ retained favourable binding (compound 7, Table 2).

All-atom GaMD simulations successfully captured spontaneous binding of the risdiplam analogues to the GA-rich DNA and RNA sequences. However, it is important to note that the GaMD free energy profiles were not fully converged, because still only few ligand binding events (insufficient sampling) were observed in the GaMD simulations. Nevertheless, relatively low-energy conformational states of each system could be identified from the simulations, which uncovered a folded double loop-like conformation induced by small-molecule binding in both DNA and RNA.

We also discovered that the binding to the GA-rich sequence is not sufficient to correct SMN2 splicing in cellular assays. Several risdiplam analogues retain the ssDNA or ssRNA-binding ability without showing any observable splicing modulation in cells (e.g., compounds 2, 5, 10, 11; Table 8). This is not completely unexpected because the GA-rich sequence is not the primary target for splicing modulation (17).

It is important to note that the dissociation constants (K_{d}) for the binding of the small molecules to either the GA-rich sequence in this study or to the 5′ splice site-U1 snRNP complex in the previous report (17) are in micromolar range, orders of magnitude higher than the EC₅₀ values of some of the active splicing modifiers in cell-based splicing assay (e.g., Table 2, entries 1-4). The discrepancy between the high cellular activity and relatively low binding affinity is actually quite common in this type of action-dependent drugs. Compared to the traditional occupation-dependent drugs (e.g., kinase inhibitors), risdiplam analogues are not required to remain bound to the mRNA once exon 7 splicing is complete. Therefore, the effective dose of risdiplam analogues in cells can be much smaller than the K_{d}. An example of this type of behavior was demonstrated by a recently optimized proteolysis targeting chimaera (PROTAC), namely ARD-266, which selectively targets and degrades androgen receptor (55). The K_{d} between ARD-266 and von Hippel-Lindau (VHL) E3 ligase is at micromolar (55). However, in the cell-based assay, ARD-266 causes 50% reduction of the androgen receptor protein level at less than 1 nM in VCaP cells, in other words, EC₅₀<0.1% K_{d} (55).

Our studies demonstrated that SMN-C2, in general, binds with higher affinity to ssDNA sequences relative to ssRNA (Table 1). In cells, most of the DNA is doubly stranded in the genome. However, ssDNAs transiently form during DNA replication. Therefore, there is a concern that the DNA-binding ability may associate with genotoxicity (11). Although one of the strongest GA-rich sequence ligands, SMN-C5, is negative in the Ames test (11), further studies are required to correlate genotoxicity and DNA-binding. In the presence of a stable TSL2 in SMN2 exon 7, the two binding sites of risdiplam analogues on SMN2 pre-mRNA exon 7, while not close together in sequence, are probably close together in space (FIG. 1). Although the GA-rich sequence is crucial in maintaining the drugs' potency for regulating splicing, binding to the GA-rich alone is not sufficient to induce SMN2 exon 7 inclusion. It is, therefore, possible that the GA-rich sequence serves as an auxiliary binding site and facilitates ligand binding to the 5′ splice site, i.e., a small-molecule delivery relay.

As previously hypothesized (15), binding to both the GA-rich sequence and to the 5′ splice site contributes to the selectivity for the risdiplam analogues. Compared to another structurally unrelated splicing modulator for SMN2 exon 7, branaplam, which only acts through binding to the 5′ splice site of the exon (56) without detectable binding to the GA-rich sequence, risdiplam analogue SMN-C3 only significantly affects splicing in 13 genes, while branaplam affects the splicing of 36 genes (15). The forkhead box M1 (FoxM1) gene is one of the 13 risdiplam-sensitive genes but lacks a GA-rich sequence (18). Compared to FoxM1, the SMN2 gene is ˜10 times more sensitive to a risdiplam analogue, RG-7800 (8), consistent with the hypothesis that the GA-rich sequence enhances the drug potency. Recently, it was discovered that another risdiplam analogue, TEC-1, with a modified ring B/C (57), has even fewer off-target effects. By changing the risdiplam's pyridopyrimidinone core in ring B/C into a quinazolinone, the FoxM1 gene splicing becomes even less sensitive to TEC-1 than risdiplam. This result underscored the possibility that sequence recognition of the risdiplam analogue can be changed by modification of the ring B/C.

### CONCLUSION

Our results reveal a new type of small molecule-RNA recognition mechanism that is relevant to the mechanism of action of a recently approved drug, risdiplam. Through molecular dynamic simulations, we revealed a new drug-inducible GAAG double loop-like structure for both DNAs and RNAs, which can be simply represented by a consecutive primary sequence of 9 nts, i.e., GAAGGAAGG. In the literature, long primary RNA sequence recognition by the small molecules is often associated with G-quadruplex formation (e.g., (GGGGCC) repeats (58)). To our knowledge, the GA-rich sequence is the first example of a consecutive RNA primary sequence of 9 or more nts that a small molecule can selectively recognize in the absence of G-quadruplexes (50).

### Example 2: RNA Binding Compounds

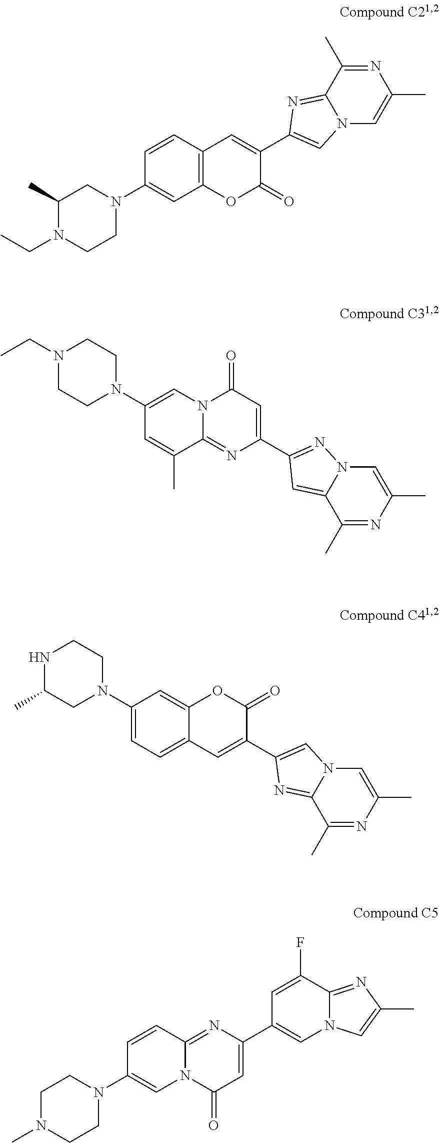

### Compound C6—(S)-tert-butyl(2-(4-(3-(6,8-dimethylimidazo[1,2-a]pyrazin-2-yl)-2-oxo-2H-chromen-7-yl)-2-methylpiperazin-1-yl)ethyl)carbamate

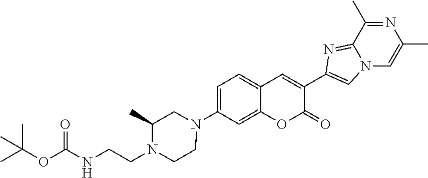 [SMILES: Cc1cn2cc(-c3cc4ccc(N5CCN(CCNC(=O)OC(C)(C)C)[C@@H](C)C5)cc4oc3=O)nc2c(C)n1]

¹H NMR (400 MHz, CDCl₃): δ 8.74 (s, 1H), 8.45 (s, 1H), 7.77 (s, 1H), 7.51 (d, J=8.8 Hz, 1H), 6.86 (dd, J=8.8, 2.4 Hz, 1H), 6.75 (d, J=2.4 Hz, 1H), 4.96 (brs, 1H), 3.59 (t, J=12 Hz, 2H), 3.31-3.20 (m, 3H), 3.03-2.92 (m, 6H), 2.67 (brs, 1H), 2.49-2.33 (m, 5H), 1.48 (s, 9H), 1.17 (d, J=6.4 Hz, 3H); ¹³C NMR (100 MHz, CDCl₃): δ 160.7, 156.0, 155.5, 153.4, 151.2, 140.1, 139.3, 138.9, 137.1, 129.3, 115.4, 114.4, 113.6, 111.9, 111.1, 100.3, 79.3, 54.8, 54.3, 52.4, 49.9, 47.5, 28.5, 20.8, 20.5, 15.8. MS-ESI (m/z) [M+H]⁺ 533.29.

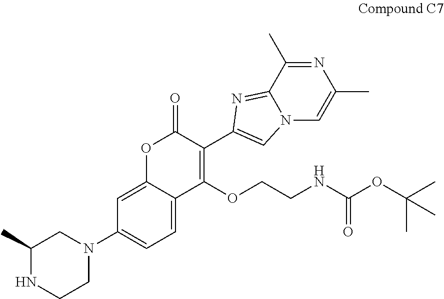 [SMILES: Cc1cn2cc(-c3c(OCCNC(=O)OC(C)(C)C)c4ccc(N5CCN[C@@H](C)C5)cc4oc3=O)nc2c(C)n1]

### Compound C8—(S)-7-(4-acetyl-3-methylpiperazin-1-yl)-3-(6,8-dimethylimidazo[1,2-a]pyrazin-2-yl)-2H-chromen-2-one

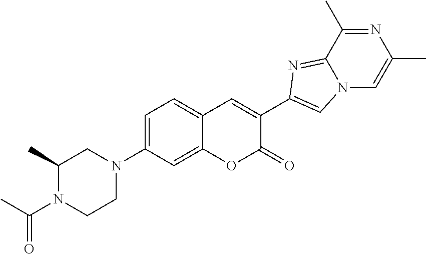 [SMILES: CC(=O)N1CCN(c2ccc3cc(-c4cn5cc(C)nc(C)c5n4)c(=O)oc3c2)C[C@@H]1C]

¹H NMR (400 MHz, CDCl₃): δ 8.77 (s, 1H), 8.47 (s, 1H), 7.79 (s, 1H), 7.55 (d, J=7.2 Hz, 1H), 6.85 (dd, J=7.4, 2.0 Hz, 1H), 6.75 (s, 1H), 3.80-3.60 (m, 4H), 3.29-3.11 (m, 3H), 2.94 (s, 3H), 2.51 (s, 3H), 2.17 (d, J=9.2 Hz, 3H), 1.27 (s, 3H). MS-ESI (m/z) [M+H]⁺ 432.27.

### Compound C10—(S)-3-(6,8-dimethylimidazo[1,2-a]pyrazin-2-yl)-7-(3-methyl-4-(prop-2-yn-1-yl)piperazin-1-yl)-2H-chromen-2-one

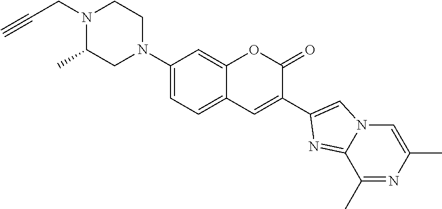 [SMILES: C#CCN1CCN(c2ccc3cc(-c4cn5cc(C)nc(C)c5n4)c(=O)oc3c2)C[C@@H]1C]

¹H NMR (400 MHz, DMSO-_{d6}): δ 8.75 (s, 1H), 8.53 (s, 1H), 8.34 (s, 1H), 7.77 (d, J=8.8 Hz, 1H), 7.06 (dd, J=9.2, 2.4 Hz, 1H), 6.93 (d, J=2.0 Hz, 1H), 3.91 (t, J=12.4 Hz, 2H), 3.61 (dd, J=17.6, 2.4 Hz, 1H), 3.43 (dd, J=17.6, 2.4 Hz, 1H), 3.16 (t, J=2.4 Hz, 1H), 3.01-2.96 (m, 1H), 2.82-2.79 (m, 1H), 2.77 (s, 3H), 2.63-2.56 (m, 2H), 2.38 (d, J=0.8 Hz, 3H), 1.07 (d, J=6.0 Hz, 3H). MS-ESI (m/z) [M+H]⁺ 428.23.

### Compound C11—(S)-3-(6,8-dimethylimidazo[1,2-a]pyrazin-2-yl)-7-(3-methyl-4-(3,6,9,12-tetraoxapentadec-14-yn-1-yl)piperazin-1-yl)-2H-chromen-2-one

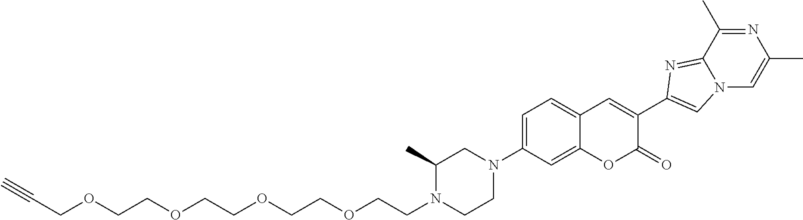 [SMILES: C#CCOCCOCCOCCOCCN1CCN(c2ccc3cc(-c4cn5cc(C)nc(C)c5n4)c(=O)oc3c2)C[C@@H]1C]

¹H NMR (400 MHz, DMSO-_{d6}): δ 8.75 (s, 1H), 8.53 (s, 1H), 8.34 (s, 1H), 7.76 (d, J=9.2 Hz, 1H), 7.05 (dd, J=9.0, 2.4 Hz, 1H), 6.91 (brs, 1H), 4.15 (d, J=2.4 Hz, 2H), 3.74 (d, J=12.4 Hz, 2H), 3.55-3.53 (m, 12H), 3.43 (t, J=2.4 Hz, 1H), 3.09-3.04 (m, 2H), 2.96-2.84 (m, 3H), 2.77 (s, 3H), 2.50-2.45 (m, 2H), 2.38 (d, J=0.8 Hz, 3H), 1.08 (d, J=6.0 Hz, 3H). MS-ESI (m/z) [M+H]⁺ 604.38.

### Compound C12—(S)-7-(4-(2-(2-azidoethoxy)ethyl)-3-methylpiperazin-1-yl)-3-(6,8-dimethylimidazo[1,2-a]pyrazin-2-yl)-2H-chromen-2-one

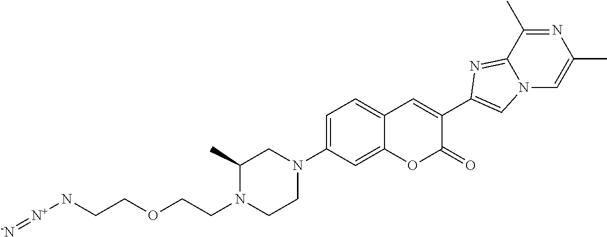

¹H NMR (400 MHz, DMSO-_{d6}): δ 8.75 (s, 1H), 8.53 (s, 1H), 8.34 (s, 1H), 7.76 (d, J=8.8 Hz, 1H), 7.05 (dd, J=9.2, 2.4 Hz, 1H), 6.91 (d, J=2.4 Hz, 1H), 3.74 (d, J=12.4 Hz, 2H), 3.63-3.57 (m, 4H), 3.42-3.40 (m, 2H), 3.09-3.04 (m, 1H), 2.98-2.87 (m, 3H), 2.79-2.73 (m, 4H), 2.48-2.45 (m, 2H), 2.38 (d, J=0.8 Hz, 3H), 1.09 (d, J=6.0 Hz, 3H). MS-ESI (m/z) [M+H]⁺ 503.20.

### Compound C13—(S)-4-bromo-3-(6,8-dimethylimidazo[1,2-a]pyrazin-2-yl)-7-(3-methylpiperazin-1-yl)-2H-chromen-2-one

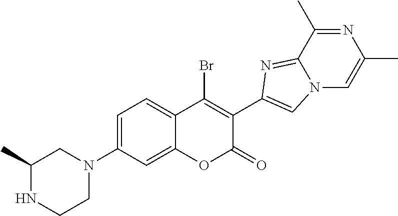

¹H NMR (400 MHz, DMSO-_{d6}): δ 8.24 (s, 1H), 8.17 (s, 1H), 7.65 (d, J=8.8 Hz, 1H), 7.04 (dd, J=9.0, 2.4 Hz, 1H), 6.91 (d, J=2.4 Hz, 1H), 3.87 (t, J=10.4 Hz, 2H), 3.02-2.99 (m, 1H), 2.83-2.76 (m, 6H), 2.49-2.45 (m, 4H), 1.07 (d, J=6.4 Hz, 3H). MS-ESI (m/z) [M+H]⁺ 470.09/468.12.

### Compound C14 (C2-dimer)—3-(6,8-dimethylimidazo[1,2-a]pyrazin-2-yl)-7-((S)-4-(2-(2-(4-(13-((S)-4-(3-(6,8-dimethylimidazo[1,2-a]pyrazin-2-yl)-2-oxo-2H-chromen-7-yl)-2-methylpiperazin-1-yl)-2,5,8,11-tetraoxatridecyl)-1H-1,2,3-triazol-1-yl)ethoxy)ethyl)-3-methylpiperazin-1-yl)-2H-chromen-2-one

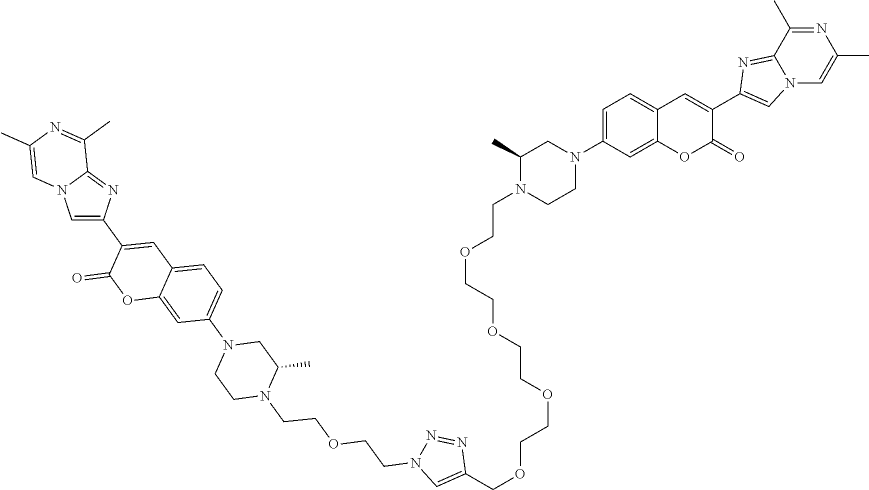

¹H NMR (400 MHz, DMSO-_{d6}): δ 8.63 (d, J=3.2 Hz, 1H), 8.45 (s, 1H), 8.26 (d, J=6.4 Hz, 2H), 8.10 (s, 1H), 7.70-7.66 (m, 3H), 6.98-6.95 (m, 3H), 6.82-6.81 (m, 2H), 4.55-4.52 (m, 4H), 3.82 (t, J=5.2 Hz, 3H), 3.68-3.65 (m, 4H), 3.56-3.49 (m, 16H), 3.01-2.96 (m, 4H), 2.92-2.81 (m, 8H), 2.73 (d, J=3.2 Hz, 3H), 2.69-2.67 (m, 2H), 2.35-2.32 (m, 7H), 1.05-1.02 (m, 6H). MS-ESI (m/z) [M+2H]⁺ (m/z=M/2+1) 552.74.

### Compound C18—3-(2-chlorophenyl)-7-(4-methylpiperazin-1-yl)-2H-chromen-2-one

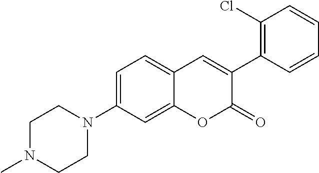 [SMILES: CN1CCN(c2ccc3cc(-c4ccccc4Cl)c(=O)oc3c2)CC1]

¹H NMR (400 MHz, CDCl₃): δ 7.62 (s, 1H), 7.48-7.46 (m, 1H), 7.42-7.40 (m, 1H), 7.35 (d, J=8.8 Hz, 1H), 7.32-7.30 (m, 2H), 6.84 (dd, J=8.8, 2.4 Hz, 1H), 6.77 (d, J=2.4 Hz, 1H), 3.40 (t, J=5.2 Hz, 4H), 2.60 (t, J=5.2 Hz, 4H), 2.38 (s, 3H); ¹³C NMR (100 MHz, CDCl₃): δ 160.7, 156.2, 153.8, 142.9, 134.6, 134.0, 131.8, 130.0, 129.7, 128.9, 126.9, 122.0, 111.9, 110.9, 101.1, 54.7, 47.6, 46.2. MS-ESI (m/z) [M+H]⁺ 355.13.

### Compound C19—3-(3-chlorophenyl)-7-(4-methylpiperazin-1-yl)-2H-chromen-2-one

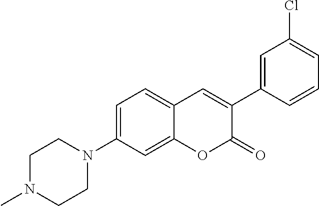 [SMILES: CN1CCN(c2ccc3cc(-c4cccc(Cl)c4)c(=O)oc3c2)CC1]

¹H NMR (500 MHz, CDCl₃): δ 7.72 (s, 1H), 7.69 (t, J=2.0 Hz, 1H), 7.60 (dt, J=7.0, 2.0 Hz, 1H), 7.38-7.33 (m, 3H), 6.84 (dd, J=9.0, 2.5 Hz, 1H), 6.75 (d, J=2.5 Hz, 1H), 3.40 (t, J=5.0 Hz, 4H), 2.60 (brs, 4H), 2.39 (s, 3H); ¹³C NMR (125 MHz, CDCl₃): δ 161.1, 155.9, 153.7, 140.8, 137.3, 134.4, 129.7, 129.0, 128.4, 128.3, 126.7, 121.8, 112.0, 111.3, 100.8, 54.7, 47.5, 46.1. MS-ESI (m/z) [M+H]355.15.

### Compound C20—3-(4-chlorophenyl)-7-(4-methylpiperazin-1-yl)-2H-chromen-2-one

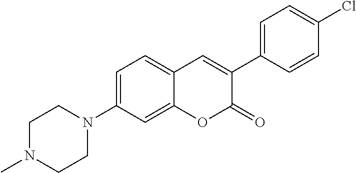 [SMILES: CN1CCN(c2ccc3cc(-c4ccc(Cl)cc4)c(=O)oc3c2)CC1]

¹H NMR (500 MHz, CDCl₃): δ 7.71 (s, 1H), 7.65 (dt, J=9.0, 2.5 Hz, 2H), 7.40-7.37 (m, 3H), 6.84 (dd, J=8.5, 2.5 Hz, 1H), 6.75 (d, J=2.5 Hz, 1H), 3.43 (brs, 4H), 2.65 (brs, 4H), 2.42 (s, 3H); ¹³C NMR (125 MHz, CDCl₃): δ 161.2, 155.8, 153.5, 140.2, 134.2, 134.0, 129.8, 128.9, 128.7, 122.2, 112.0, 111.4, 100.9, 54.6, 47.4, 46.0. MS-ESI (m/z) [M+H]⁺ 355.14.

### Compound C21—3-(2-chlorophenyl)-4-methyl-7-(4-methylpiperazin-1-yl)-2H-chromen-2-one

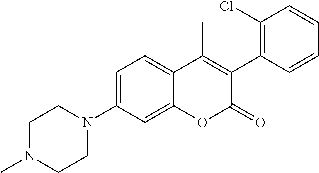 [SMILES: Cc1c(-c2ccccc2Cl)c(=O)oc2cc(N3CCN(C)CC3)ccc12]

¹H NMR (400 MHz, DMSO-_{d6}): δ 7.66 (d, J=8.8 Hz, 1H), 7.60-7.58 (m, 1H), 7.48-7.42 (m, 2H), 7.39-7.36 (m, 1H), 7.04 (dd, J=9.0, 2.4 Hz, 1H), 6.89 (d, J=2.4 Hz, 1H), 3.38 (t, J=4.8 Hz, 4H), 2.46 (t, J=4.8 Hz, 4H), 2.24 (s, 3H), 2.12 (s, 3H). MS-ESI (m/z) [M+H]⁺ 369.13.

### Compound C22—3-(3-chlorophenyl)-4-methyl-7-(4-methylpiperazin-1-yl)-2H-chromen-2-one

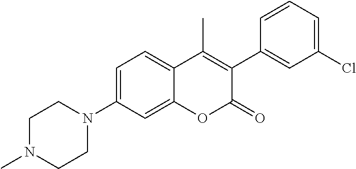 [SMILES: Cc1c(-c2cccc(Cl)c2)c(=O)oc2cc(N3CCN(C)CC3)ccc12]

¹H NMR (500 MHz, CDCl₃): δ 7.51 (d, J=9.0 Hz, 1H), 7.39-7.34 (m, 2H), 7.30 (t, J=2.0 Hz, 1H), 7.19 (dt, J=6.5, 2.0 Hz, 1H), 6.86 (dd, J=9.0, 2.5 Hz, 1H), 6.76 (d, J=2.5 Hz, 1H), 3.41 (t, J=5.0 Hz, 4H), 2.64 (brs, 4H), 2.41 (s, 3H), 2.25 (s, 3H); ¹³C NMR (125 MHz, CDCl₃): δ 161.5, 154.7, 153.5, 148.6, 136.9, 134.3, 130.6, 129.7, 128.8, 128.2, 126.1, 121.9, 112.0, 111.7, 101.3, 54.7, 47.5, 46.1, 16.6. MS-ESI (m/z) [M+H]⁺ 369.17.

### Compound C23—3-(4-chlorophenyl)-4-methyl-7-(4-methylpiperazin-1-yl)-2H-chromen-2-one

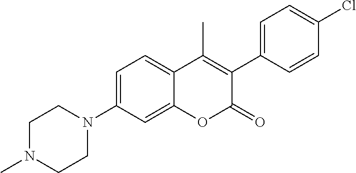 [SMILES: Cc1c(-c2ccc(Cl)cc2)c(=O)oc2cc(N3CCN(C)CC3)ccc12]

¹H NMR (500 MHz, CDCl₃): δ 7.50 (d, J=8.5 Hz, 1H), 7.41 (dt, J=8.5, 2.0 Hz, 2H), 7.24 (dt, J=8.5, 2.0 Hz, 2H), 6.86 (dd, J=9.0, 2.5 Hz, 1H), 6.76 (d, J=2.0 Hz, 1H), 3.39 (t, J=5.0 Hz, 4H), 2.61 (brs, 4H), 2.39 (s, 3H), 2.25 (s, 3H); ¹³C NMR (125 MHz, CDCl₃): δ 161.6, 154.7, 153.4, 148.4, 134.0, 133.5, 132.0, 128.7, 126.1, 122.0, 112.1, 111.7, 101.3, 54.7, 47.6, 46.1, 16.5. MS-ESI (m/z) [M+H]⁺ 369.15.

### Compound C24—3-(2-chlorophenyl)-5-fluoro-7-(4-methylpiperazin-1-yl)-2H-chromen-2-one

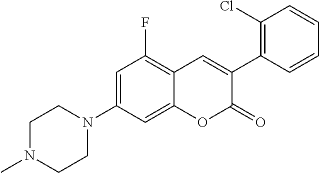 [SMILES: CN1CCN(c2cc(F)c3cc(-c4ccccc4Cl)c(=O)oc3c2)CC1]

¹H NMR (400 MHz, DMSO-_{d6}): δ 7.90 (s, 1H), 7.57-7.55 (m, 1H), 7.52-7.49 (m, 1H), 7.48-7.40 (m, 2H), 6.74 (dd, J=13.8, 2.4 Hz, 1H), 6.79 (d, J=2.4 Hz, 1H), 3.42 (t, J=5.2 Hz, 4H), 2.43 (t, J=5.2 Hz, 4H), 2.23 (s, 3H). MS-ESI (m/z) [M+H]⁺ 373.11.

### Compound C25—3-(3-chlorophenyl)-5-fluoro-7-(4-methylpiperazin-1-yl)-2H-chromen-2-one

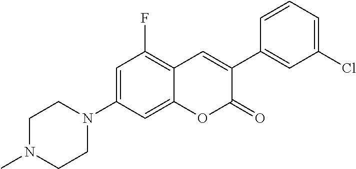 [SMILES: CN1CCN(c2cc(F)c3cc(-c4cccc(Cl)c4)c(=O)oc3c2)CC1]

¹H NMR (500 MHz, CDCl₃): δ 7.90 (s, 1H), 7.69 (t, J=2.0 Hz, 1H), 7.59 (dt, J=6.5, 2.0 Hz, 1H), 7.37-7.34 (m, 2H), 6.54-6.51 (m, 2H), 3.37 (t, J=5.0 Hz, 4H), 2.56 (t, J=5.0 Hz, 4H), 2.37 (s, 3H); ¹³C NMR (125 MHz, CDCl₃): δ 161.0, 160.5, 159.0, 156.0, 153.7, 137.0, 134.5, 133.9, 129.8, 128.5, 126.7, 121.5, 101.3, 97.5, 96.4, 54.6, 47.4, 46.2. MS-ESI (m/z) [M+H]⁺ 373.15.

### Compound C26—3-(4-chlorophenyl)-5-fluoro-7-(4-methylpiperazin-1-yl)-2H-chromen-2-one

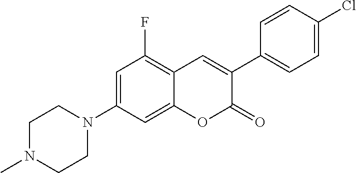 [SMILES: CN1CCN(c2cc(F)c3cc(-c4ccc(Cl)cc4)c(=O)oc3c2)CC1]

¹H NMR (500 MHz, CDCl₃): δ 7.87 (s, 1H), 7.64 (dt, J=9.0, 2.5 Hz, 2H), 7.39 (dt, J=9.0, 2.5 Hz, 2H), 6.53-6.50 (m, 1H), 3.37 (t, J=5.0 Hz, 4H), 2.56 (t, J=5.0 Hz, 4H), 2.37 (s, 3H); ¹³C NMR (125 MHz, CDCl₃): δ 160.9, 160.7, 159.0, 155.9, 153.6, 134.4, 133.7, 133.3, 129.8, 128.7, 121.7, 101.4, 97.5, 96.4, 54.6, 47.4, 46.2. MS-ESI (m/z) [M+H]⁺ 373.13.

### Compound C27—3-(3,4-dimethoxyphenyl)-7-(4-methylpiperazin-1-yl)-2H-chromen-2-one

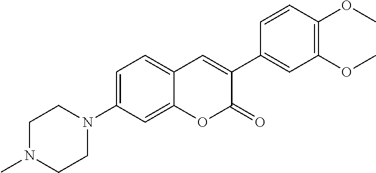

¹H NMR (400 MHz, DMSO-_{d6}): δ 8.10 (s, 1H), 7.56 (d, J=8.8 Hz, 1H), 7.33-7.30 (m, 2H), 7.02 (dd, J=8.6, 2.0 Hz, 1H), 6.87 (d, J=2.4 Hz, 1H), 3.81 (s, 3H), 3.80 (s, 3H), 3.37 (t, J=5.2 Hz, 4H), 2.45 (t, J=5.2 Hz, 4H), 2.24 (s, 3H). MS-ESI (m/z) [M+H]381.22.

### Compound C28—3-(6-methoxyimidazo[1,2-a]pyrazin-2-yl)-7-(piperazin-1-yl)-2H-chromen-2-one

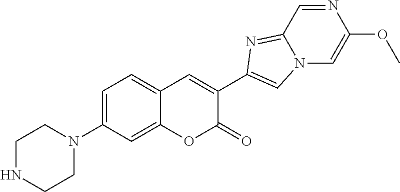 [SMILES: COc1cn2cc(-c3cc4ccc(N5CCNCC5)cc4oc3=O)nc2cn1]

¹H NMR (500 MHz, DMSO-_{d6}): δ 8.82 (s, 1H), 8.76 (s, 1H), 8.60 (s, 1H), 8.31 (d, J=1.5 Hz, 1H), 7.70 (d, J=9.0 Hz, 1H), 7.02 (dd, J=9.0, 2.5 Hz, 1H), 6.85 (d, J=2 Hz, 1H), 3.87 (s, 3H), 3.32 (t, J=5 Hz, 4H), 2.83 (t, J=5 Hz, 4H); ¹³C NMR (125 MHz, DMSO-_{d6}): δ 159.7, 155.3, 154.0, 152.7, 141.6, 139.7, 138.8, 138.3, 129.9, 113.7, 111.7, 109.9, 102.4, 99.1, 55.0, 47.7, 45.3. MS-ESI (m/z) [M+H]378.17.

### Compound C29—3-(imidazo[1,2-a]pyridin-2-yl)-7-(piperazin-1-yl)-2H-chromen-2-one

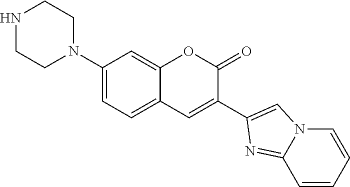 [SMILES: O=c1oc2cc(N3CCNCC3)ccc2cc1-c1cn2ccccc2n1]

¹H NMR (500 MHz, DMSO-_{d6}): δ 8.72 (s, 1H), 8.62 (d, J=7.0 Hz, 1H), 8.52 (s, 1H), 7.69 (d, J=9.0 Hz, 1H), 7.54 (d, J=9.0 Hz, 1H), 7.29-7.25 (m, 1H), 7.01 (dd, J=9.0, 2.5 Hz, 1H), 6.90-6.85 (m, 2H), 3.29 (t, J=5 Hz, 4H), 2.82 (t, J=5 Hz, 4H); ¹³C NMR (125 MHz, DMSO-_{d6}): δ 159.7, 155.0, 153.8, 144.2, 138.6, 138.5, 129.6, 127.3, 125.6, 116.1, 114.6, 112.2, 111.7, 110.2, 99.2, 48.0, 45.4. MS-ESI (m/z) [M+H]⁺ 347.14.

### Compound C30—3-(7-fluoroimidazo[1,2-a]pyridin-2-yl)-7-(piperazin-1-yl)-2H-chromen-2-one

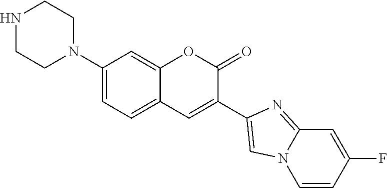 [SMILES: O=c1oc2cc(N3CCNCC3)ccc2cc1-c1cn2ccc(F)cc2n1]

¹H NMR (400 MHz, DMSO-_{d6}): δ 8.73-8.69 (m, 2H), 8.54 (s, 1H), 7.73 (d, J=8.8 Hz, 1H), 7.40 (dd, J=10.0, 2.8 Hz, 1H), 7.06-6.97 (m, 2H), 6.91 (d, J=2.4 Hz, 1H), 3.40 (t, J=5.2 Hz, 4H), 2.96 (t, J=5.2 Hz, 4H). MS-ESI (m/z) [M+H]⁺ 365.17.

### Compound C31—3-(6-fluoroimidazo[1,2-a]pyridin-2-yl)-7-(piperazin-1-yl)-2H-chromen-2-one

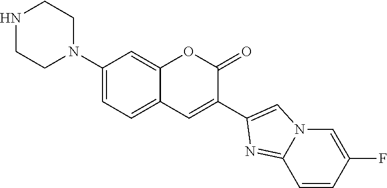 [SMILES: O=c1oc2cc(N3CCNCC3)ccc2cc1-c1cn2cc(F)ccc2n1]

¹H NMR (400 MHz, DMSO-_{d6}): δ 8.86-8.45 (m, 1H), 8.72 (s, 1H), 8.55 (s, 1H), 7.71 (d, J=9.2 Hz, 1H), 7.62 (q, J=5.2 Hz, 1H), 7.41-7.35 (m, 1H), 7.03 (dd, J=8.8, 2.4 Hz, 1H), 6.87 (d, J=2.4 Hz, 1H), 3.32 (t, J=5.2 Hz, 4H), 2.85 (t, J=5.2 Hz, 4H). MS-ESI (m/z) [M+H]⁺ 365.19.

### Compound C32—3-(7-chloroimidazo[1,2-a]pyridin-2-yl)-7-(piperazin-1-yl)-2H-chromen-2-one

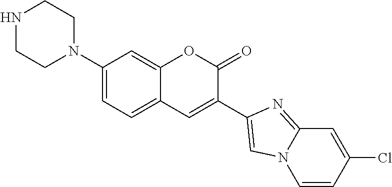 [SMILES: O=c1oc2cc(N3CCNCC3)ccc2cc1-c1cn2ccc(Cl)cc2n1]

¹H NMR (400 MHz, DMSO-_{d6}): δ 8.71 (s, 1H), 8.67 (d, J=7.6 Hz, 1H), 8.57 (s, 1H), 7.72-7.70 (m, 2H), 7.04-6.99 (m, 2H), 6.87 (d, J=2.4 Hz, 1H), 3.32 (t, J=5.2 Hz, 4H), 2.85 (t, J=5.2 Hz, 4H). MS-ESI (m/z) [M+H]381.12.

### Compound C33—3-(6-chloroimidazo[1,2-a]pyridin-2-yl)-7-(piperazin-1-yl)-2H-chromen-2-one

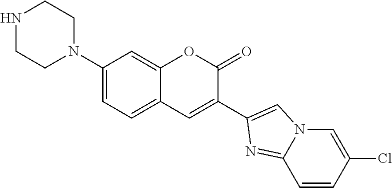 [SMILES: O=c1oc2cc(N3CCNCC3)ccc2cc1-c1cn2cc(Cl)ccc2n1]

¹H NMR (400 MHz, DMSO-_{d6}): δ 8.93 (dd, J=2.0, 0.8 Hz, 1H), 8.75 (s, 1H), 8.55 (s, 1H), 7.73 (d, J=9.2 Hz, 1H), 7.61 (d, J=9.6 Hz, 1H), 7.34 (dd, J=9.4, 2.4 Hz, 1H), 7.05 (dd, J=8.8, 2.4 Hz, 1H), 6.92 (d, J=2.4 Hz, 1H), 3.41 (t, J=5.2 Hz, 4H), 2.97 (t, J=5.2 Hz, 4H). MS-ESI (m/z) [M+H]⁺ 381.15.

### Compound C34—3-(7-methoxyimidazo[1,2-a]pyridin-2-yl)-7-(piperazin-1-yl)-2H-chromen-2-one

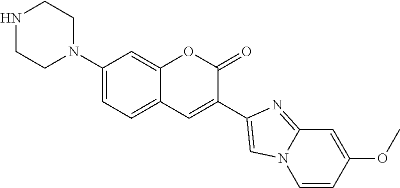 [SMILES: COc1ccn2cc(-c3cc4ccc(N5CCNCC5)cc4oc3=O)nc2c1]

¹H NMR (400 MHz, DMSO-_{d6}): δ 8.63 (s, 1H), 8.47 (dd, J=7.4, 0.8 Hz, 1H), 8.35 (s, 1H), 7.68 (d, J=8.8 Hz, 1H), 7.02 (dd, J=8.8, 2.4 Hz, 1H), 6.88 (dd, J=8.2, 2.4 Hz, 1H), 6.62 (dd, J=7.4, 2.4 Hz, 1H), 3.86 (s, 3H), 3.34 (t, J=5.2 Hz, 4H), 2.89 (t, J=5.2 Hz, 4H). MS-ESI (m/z) [M+H]⁺ 377.21.

### Compound C35—3-(6-methoxyimidazo[1,2-a]pyridin-2-yl)-7-(piperazin-1-yl)-2H-chromen-2-one

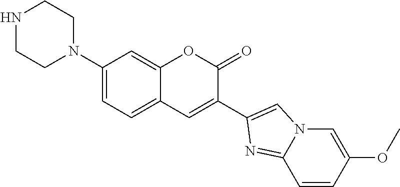 [SMILES: COc1ccc2nc(-c3cc4ccc(N5CCNCC5)cc4oc3=O)cn2c1]

¹H NMR (400 MHz, DMSO-_{d6}): δ 8.68 (s, 1H), 8.46 (s, 1H), 8.37 (dd, J=2.4, 0.8 Hz, 1H), 7.69 (d, J=9.2 Hz, 1H), 7.47 (d, J=9.6 Hz, 1H), 7.08 (dd, J=10.0, 2.4 Hz, 1H), 7.02 (dd, J=9.2, 2.4 Hz, 1H), 6.87 (d, J=2.4 Hz, 1H), 3.80 (s, 3H), 3.32 (t, J=5.2 Hz, 4H), 2.87 (t, J=5.2 Hz, 4H). MS-ESI (m/z) [M+H]⁺ 377.21.

### Compound C36—7-(piperazin-1-yl)-3-(7-(trifluoromethyl)imidazo[1,2-a]pyridin-2-yl)-2H-chromen-2-one

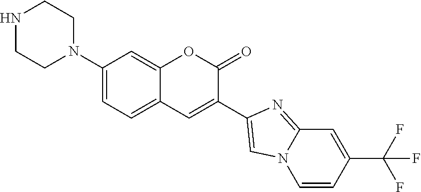 [SMILES: O=c1oc2cc(N3CCNCC3)ccc2cc1-c1cn2ccc(C(F)(F)F)cc2n1]

¹H NMR (400 MHz, DMSO-_{d6}): δ 8.86 (d, J=7.2 Hz, 1H), 8.77 (s, 1H), 8.75 (s, 1H), 8.02 (s, 1H), 7.74 (d, J=9.2 Hz, 1H), 7.20 (dd, J=7.2, 2.0 Hz, 1H), 7.04 (dd, J=8.8, 2.4 Hz, 1H), 6.89 (d, J=2.4 Hz, 1H), 2.88 (t, J=5.2 Hz, 4H). MS-ESI (m/z) [M+H]⁺ 415.17.

### Compound C37—7-(piperazin-1-yl)-3-(6-(trifluoromethyl)imidazo[1,2-a]pyridin-2-yl)-2H-chromen-2-one

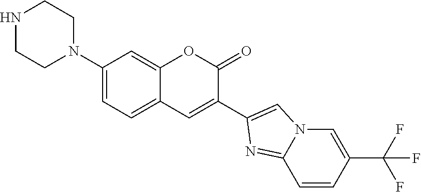 [SMILES: O=c1oc2cc(N3CCNCC3)ccc2cc1-c1cn2cc(C(F)(F)F)ccc2n1]

¹H NMR (400 MHz, DMSO-_{d6}): δ 9.33-9.32 (m, 1H), 8.77 (s, 1H), 8.69 (s, 1H), 7.76-7.72 (m, 2H), 7.51 (dd, J=9.6, 2.0 Hz, 1H), 7.04 (dd, J=8.8, 2.4 Hz, 1H), 6.89 (d, J=2.4 Hz, 1H), 2.89 (t, J=5.2 Hz, 4H). MS-ESI (m/z) [M+H]⁺ 415.18.

### Compound C38

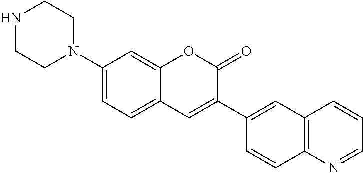 [SMILES: O=c1oc2cc(N3CCNCC3)ccc2cc1-c1ccc2ncccc2c1]

¹H NMR (400 MHz, DMSO-d6) δ 8.92 (dd, J=4.2, 1.7 Hz, 1H), 8.46-8.39 (m, 1H), 8.38 (d, J=2.0 Hz, 1H), 8.34 (s, 1H), 8.16-8.03 (m, 2H), 7.03 (dd, J=8.9, 2.4 Hz, 1H), 6.89 (d, J=2.3 Hz, 1H), 3.31-3.21 (m, 4H), 2.89-2.80 (m, 4H). MS-ESI (m/z) [M+2H]⁺/2 179.65.

### Compound C39

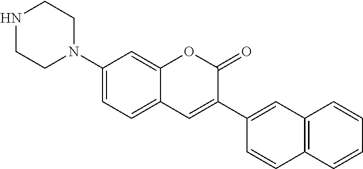 [SMILES: O=c1oc2cc(N3CCNCC3)ccc2cc1-c1ccc2ccccc2c1]

¹H NMR (400 MHz, DMSO-d6) δ 8.30 (d, J=1.8 Hz, 1H), 8.28 (s, 1H), 7.98-7.91 (m, 3H), 7.85 (dd, J=8.6, 1.8 Hz, 1H), 7.60 (d, J=8.9 Hz, 1H), 7.58-7.51 (m, 2H), 7.01 (dd, J=8.9, 2.4 Hz, 1H), 6.87 (d, J=2.3 Hz, 1H), 3.30-3.22 (m, 4H), 2.87-2.78 (m, 4H). MS-ESI (m/z) [M+H]⁺ 357.17.

### Compound C40—3-(6,8-dimethylimidazo[1,2-a]pyrazin-2-yl)-7-(piperazin-1-yl)-2H-chromen-2-one

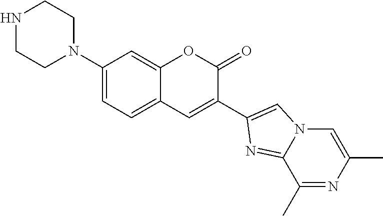 [SMILES: Cc1cn2cc(-c3cc4ccc(N5CCNCC5)cc4oc3=O)nc2c(C)n1]

¹H NMR (400 MHz, DMSO-_{d6}): δ 8.74 (s, 1H), 8.53 (s, 1H), 8.33 (s, 1H), 7.75 (d, J=8.8 Hz, 1H), 7.02 (dd, J=9.0, 2.4 Hz, 1H), 6.87 (d, J=2.4 Hz, 1H), 3.32 (t, J=5.2 Hz, 4H), 2.84 (t, J=5.2 Hz, 4H), 2.76 (s, 3H), 2.38 (d, J=0.8 Hz, 3H).

### Compound C41—3-(6,8-dimethylimidazo[1,2-a]pyrazin-2-yl)-7-(4-methylpiperazin-1-yl)-2H-chromen-2-one

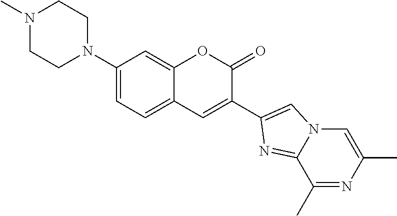

¹H NMR (400 MHz, DMSO-_{d6}): δ 8.75 (s, 1H), 8.53 (s, 1H), 8.33 (s, 1H), 7.77 (d, J=8.8 Hz, 1H), 7.05 (dd, J=8.8, 2.4 Hz, 1H), 6.91 (d, J=2.4 Hz, 1H), 3.41 (t, J=5.2 Hz, 4H), 2.77 (s, 3H), 2.45 (t, J=5.2 Hz, 4H), 2.38 (d, J=1.2 Hz, 3H), 2.24 (s, 3H).

### Compound C42—(S)-3-(6-methoxy-8-methylimidazo[1,2-a]pyrazin-2-yl)-7-(3-methylpiperazin-1-yl)-2H-chromen-2-one

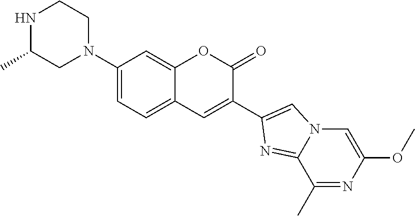

¹H NMR (400 MHz, DMSO-_{d6}): δ 8.73 (s, 1H), 8.57 (s, 1H), 8.15 (d, J=0.8 Hz, 1H), 7.75 (d, J=9.2 Hz, 1H), 7.03 (dd, J=9.2, 2.4 Hz, 1H), 6.88 (d, J=2.0 Hz, 1H), 3.85 (s, 3H), 3.83-3.81 (m, 2H), 2.99-2.96 (m, 1H), 2.79-2.74 (m, 5H), 2.45-2.39 (m, 2H), 1.05 (d, J=6.4 Hz, 3H).

### Compound C43—(S)-3-(8-methylimidazo[1,2-a]pyrazin-2-yl)-7-(3-methylpiperazin-1-yl)-2H-chromen-2-one

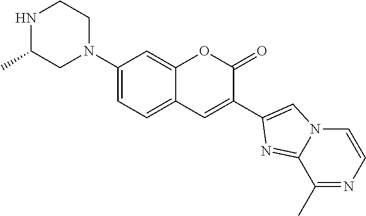 [SMILES: Cc1nccn2cc(-c3cc4ccc(N5CCN[C@@H](C)C5)cc4oc3=O)nc12]

¹H NMR (400 MHz, DMSO-_{d6}): δ 8.76 (s, 1H), 8.63 (s, 1H), 8.51 (dd, J=4.8, 0.8 Hz, 1H), 7.77-7.73 (m, 2H), 7.03 (dd, J=8.8, 2.4 Hz, 1H), 6.88 (d, J=2.4 Hz, 1H), 3.83 (t, J=9.6 Hz, 2H), 2.99-2.96 (m, 1H), 2.79 (s, 3H), 2.78-2.74 (m, 2H), 2.45-2.39 (m, 2H), 1.05 (d, J=6.4 Hz, 3H).

### Compound C44—2-(2-methylimidazo[1,2-a]pyridin-6-yl)-7-(4-methylpiperazin-1-yl)-4H-pyrido[1,2-a]pyrimidin-4-one

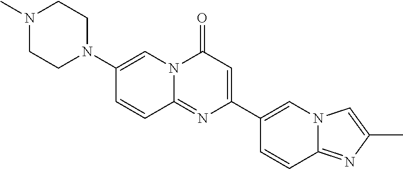

¹H NMR (400 MHz, CDCl₃): δ 9.00 (q, J=0.8 Hz, 1H), 8.47 (t, J=2.0 Hz, 1H), 7.79 (dd, J=9.4, 2.0 Hz, 1H), 7.70-7.66 (m, 3H), 7.46 (t, J=0.8 Hz, 1H), 6.83 (s, 1H), 3.50 (brs, 4H), 2.94 (brs, 4H), 2.60 (s, 3H), 2.52 (d, J=0.8 Hz, 3H).

### Compound C45—2-(2,8-dimethylimidazo[1,2-a]pyridin-6-yl)-7-(4-methylpiperazin-1-yl)-4H-pyrido[1,2-a]pyrimidin-4-one

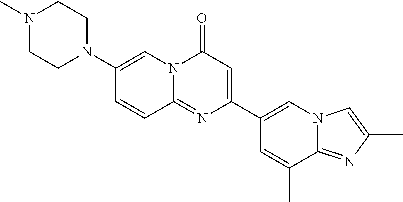

¹H NMR (400 MHz, CDCl₃): δ 8.85 (q, J=0.8 Hz, 1H), 8.47 (d, J=2.4 Hz, 1H), 7.73-7.66 (m, 2H), 7.57 (brs, 1H), 7.46 (d, J=1.2 Hz, 1H), 6.84 (s, 1H), 3.36 (t, J=5.6 Hz, 4H), 2.71 (brs, 7H), 2.54 (d, J=0.8 Hz, 3H), 2.46 (s, 3H).

### Compound C46—2-(2-methylimidazo[1,2-b]pyridazin-6-yl)-7-(4,7-diazaspiro[2.5]octan-7-yl)-4H-pyrido[1,2-a]pyrimidin-4-one

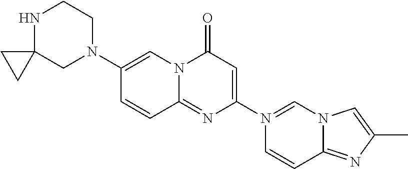

¹H NMR (400 MHz, CDCl₃): δ 8.47 (d, J=2.8 Hz, 1H), 8.12 (d, J=9.2 Hz, 1H), 7.93 (dd, J=9.2 Hz, 0.8 Hz, 1H), 7.84 (t, J=0.8 Hz, 1H), 7.76-7.66 (m, 2H), 7.40 (s, 1H), 3.40-3.38 (m, 2H), 3.30-3.27 (m, 2H), 3.18 (s, 3H), 2.55 (d, J=0.8 Hz, 3H), 0.98 (t, J=6.0 Hz, 2H), 0.76 (t, J=6.0 Hz, 2H).

### Example 3: Bifunctional Compounds

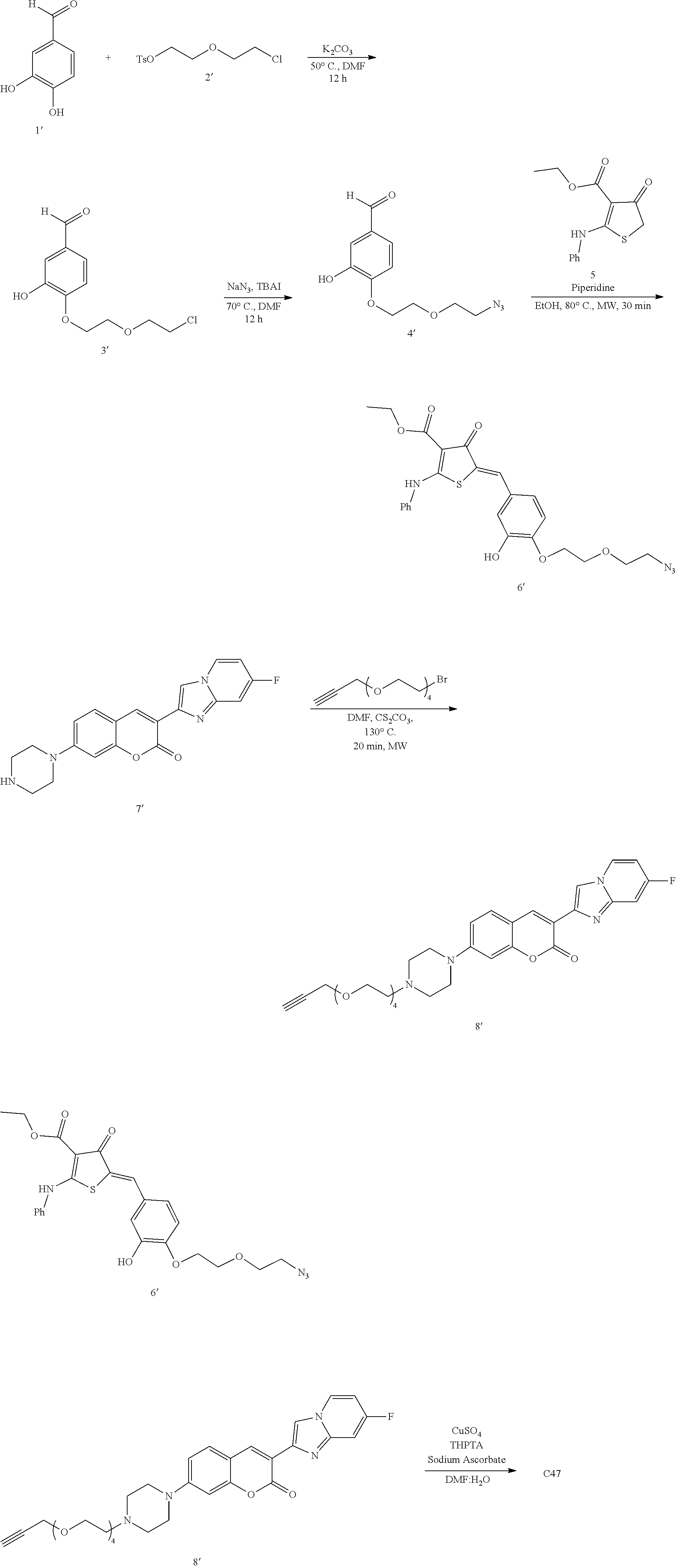

Experimental procedure for the preparation of C47: Aldehyde 1′ (0.1 g, 0.724 mmol) in DMF (2 mL) was added chloro compound (0.19 g, 0.724 mmol) and potassium carbonate (0.1 g, 0.724 mmol). The reaction mixture is stirred at 50° C. for overnight. Added water to the reaction mixture, extracted with diethyl ether. The organic phase was washed with brine solution, dried (Na₂SO₄) and concentrated in vacuo. The compound 2′ is pure enough for the next step (Colorless oil, 0.15 g, 85%). Compound 2′ (0.15 g, 0.559 mmol) in DMF (2 mL) was added Sodium azide (0.06 g, 0.832 mmol) and Tetrabutyl ammonium iodide (0.228 g, 0.11 mmol). The reaction mixture is stirred at 70° C. for overnight. Added water to the reaction mixture, extracted with diethyl ether. The organic phase was washed with brine solution, dried (Na₂SO₄) and concentrated in vacuo. The compound 3′ is purified using 18% EtOAc/Hexane system, Yellow oil, 0.082 g, 57%. Compound 3′ (0.05 g, 0.199 mmol) in ethanol was added Ethyl 4-oxo-2-(phenylamino)-4,5-dihydro thiophene-3-carboxylate 5′ (0.057 g, 0.218 mmol) and piperidine (3 uL, 0.029 mmol). The reaction mixture is heated under microwave irradiation (100° C. for 45 min). Evaporated the solvent and added ethanol, heated to 50° C. and filtered and washed with cold ethanol. Yellow solid, 0.055 g (57%). Fluoro Compound 7′ (0.05 g, 0.137 mmol) in DMF was added Cs₂CO₃ and Propargyl-PEG-4-Br and the reaction mixture is heated at 130° C. for 20 min under microwave irradiation. Yellow solids were formed, added ice water, filtered, and washed again with water and dried. The compound 8′ is purified using 5% MeOH/DCM system, Yellow solid, 0.047 g, 72%. Alkyne (1.25 mg, 0.002 mmol) in DMSO was added azide (1.0 mg, 0.002 mmol) and formed the clear solution. Sodium ascorbate (100 mM, 20 μL, 0.002 mmol) THPTA (100 mM, 20 μL, 0.002 mmol) and CuSO4 (100 mM, 20 μL, 0.002 mmol) was added. Close the vial, evacuated, and refilled with N2 three times and stirred for overnight. Evaporated the solvent and purified by 3% MeOH/DCM system, Yellow solid, 1.1 mg, 72%

### Compound C47

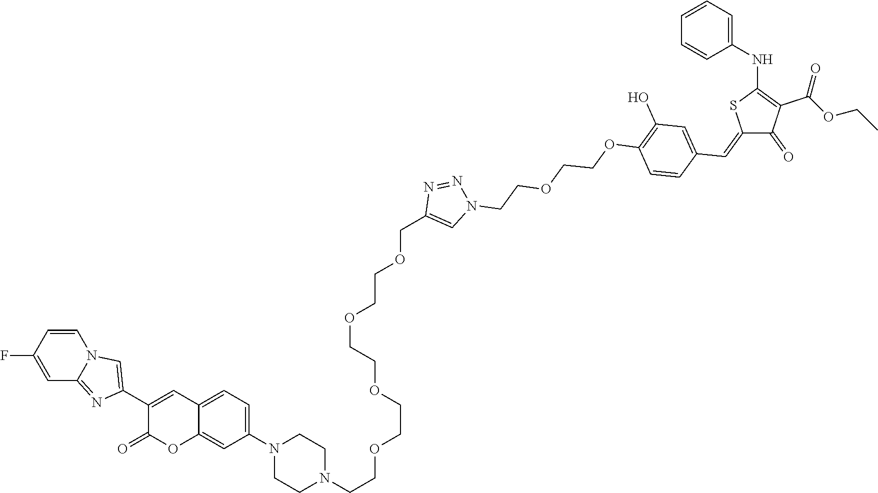 [SMILES: CCOC(=O)C1=C(Nc2ccccc2)S/C(=C\c2ccc(OCCOCCn3cc(COCCOCCOCCOCCN4CCN(c5ccc6cc(-c7cn8ccc(F)cc8n7)c(=O)oc6c5)CC4)nn3)c(O)c2)C1=O]

¹H NMR (400 MHz, Chloroform-d) δ 11.51 (s, 1H), 8.67 (s, 1H), 8.47 (s, 1H), 8.12 (t, J=6.5 Hz, 2H), 7.87 (s, 1H), 7.72 (s, 1H), 7.56-7.46 (m, 4H), 7.41 (dd, J=7.9, 3.5 Hz, 4H), 7.17 (d, J=2.2 Hz, 1H), 7.06-6.99 (m, 2H), 6.88 (d, J=8.4 Hz, 1H), 6.84-6.79 (m, 2H), 6.76 (d, J=2.4 Hz, 1H), 6.75-6.69 (m, 2H), 4.66-4.54 (m, 4H), 4.48-4.37 (m, 4H), 4.18 (t, J=4.4 Hz, 4H), 3.94 (t, J=4.9 Hz, 4H), 3.84 (t, J=4.4 Hz, 4H), 3.73-3.59 (m, 4H), 2.38 (t, J=7.6 Hz, 2H), 1.47 (t, J=7.1 Hz, 2H), 0.97-0.86 (m, 3H).

### Compound C9—(S,Z)-ethyl 5-(4-(2-(2-(4-(13-(4-(3-(6,8-dimethylimidazo[1,2-a]pyrazin-2-yl)-2-oxo-2H-chromen-7-yl)-2-methylpiperazin-1-yl)-2,5,8,11-tetraoxatridecyl)-1H-1,2,3-triazol-1-yl)ethoxy)ethoxy)-3-hydroxybenzylidene)-4-oxo-2-(phenylamino)-4,5-dihydrothiophene-3-carboxylate

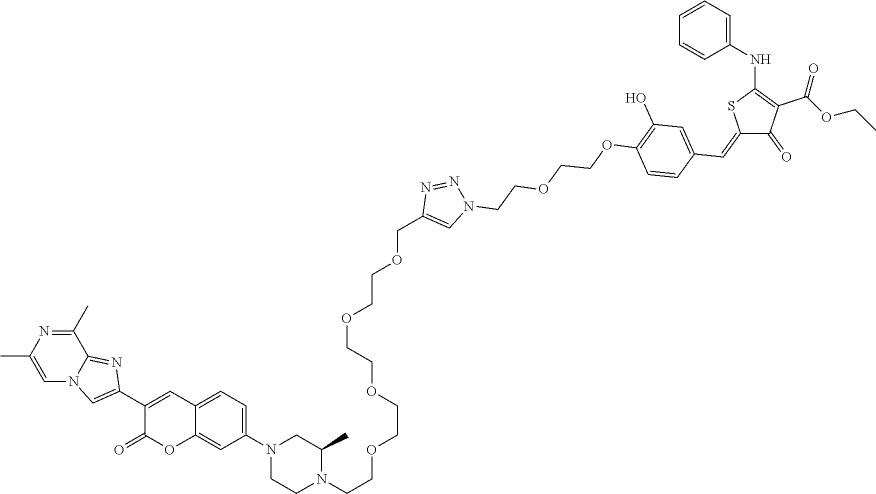 [SMILES: CCOC(=O)C1=C(Nc2ccccc2)S/C(=C\c2ccc(OCCOCCn3cc(COCCOCCOCCOCCN4CCN(c5ccc6cc(-c7cn8cc(C)nc(C)c8n7)c(=O)oc6c5)C[C@H]4C)nn3)c(O)c2)C1=O]

¹H NMR (400 MHz, DMSO-_{d6}): δ 8.74 (s, 1H), 8.53 (s, 1H), 8.33 (s, 1H), 8.06 (s, 1H), 7.75 (d, J=8.8 Hz, 1H), 7.35 (brs, 4H), 7.03 (d, J=9.2, Hz, 2H), 6.92-6.86 (m, 5H), 4.55-4.48 (m, 4H), 4.05 (brs, 2H), 3.88 (t, J=5.2 Hz, 3H), 3.74-3.73 (m, 4H), 3.53-3.48 (m, 16H), 2.94-2.83 (m, 4H), 2.77 (s, 3H), 2.69-2.67 (m, 1H), 2.38 (d, J=1.2 Hz, 3H), 2.35-2.33 (m, 2H), 1.06 (d, J=6.0 Hz, 3H). MS-ESI (m/z) [M+2H]⁺ (m/z=M/2+1) 550.94.

### Preparation of Compound C64

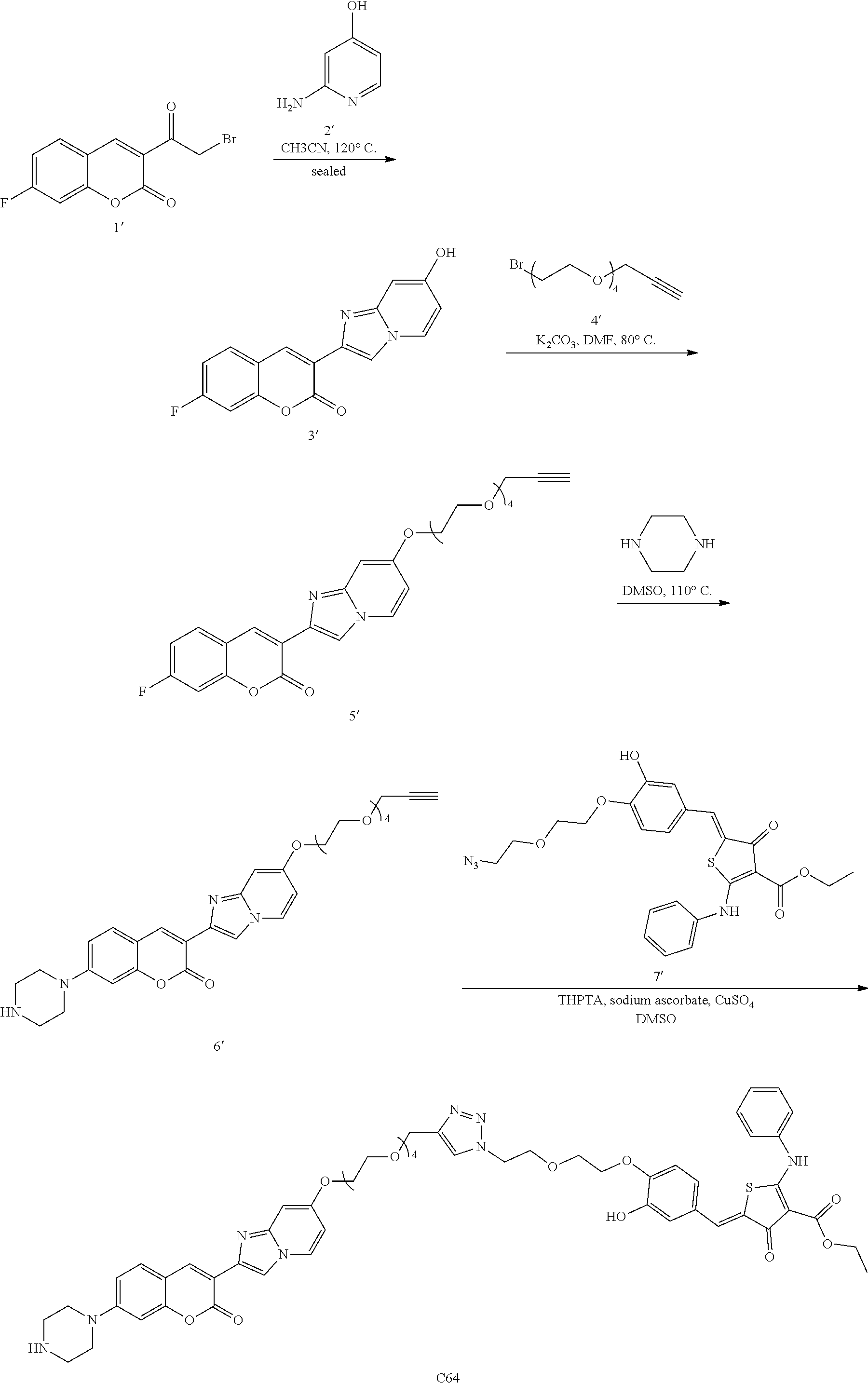

Step 1: Compounds 1′ (285 mg, 1 mmol) and 2′ (120 mg, 1.1 mmol) were dissolved in CH₃CN in a sealed tube, the mixture was heated at 120° C. for 30 min. Precipitate was filtered and the solid was washed with CH₃CN to give the pure product as a yellow solid (100 mg).

Step 2: Compounds 3′ (45 mg, 0.15 mmol) and 4′ (45 mg, 0.15 mmol) were dissolved in DMF and K₂CO₃ was added. The mixture was heated at 100° C. for 3 h. DMF was removed under vacuum. The residue was purified by flash column with 0-5% MeOH in DCM to give 32 mg oily product.

Step 3: Compound 5′ (32 mg, 0.06 mmol) and piperazine (15 mg, 0.18 mmol) were dissolved in DMSO. The mixture was heated at 120° C. for 2 h. DMSO was removed under vacuum. The residue was purified by falsh column with 0-15% MeOH in DCM to give 16 mg product.

Step 4: Compounds 6′ (6 mg, 0.01 mmol) and 7′ (5 mg, 0.01 mmol) were dissolved in DMSO and sodium ascorbate (2 mg, 0.01 mmol), THPTA (4 mg, 0.01 mmol), CuSO4 (2 mg, 0.01 mmol) were added. The mixture was stirred under nitrogen at room temperature overnight. DMSO was removed under vacuum. The residue was purified by column with 0-20% MeOH in DCM. The yellow product was further purified by HPLC to give 1 mg pure product.

¹H NMR (400 MHz, DMSO-_{d6}) δ 8.62 (s, 1H), 8.45 (d, J=8 Hz, 1H), 8.34 (s, 1H), 8.04 (s, 1H), 7.67 (d, J=8 Hz, 1H), 7.53-7.38 (m, 6H), 7.02 (d, J=8 Hz, 1H), 6.97-6.88 (m, 5H), 6.62 (dd, J=4, 8 Hz, 1H), 4.53 (t, J=4 Hz, 2H), 4.47 (s, 2H), 4.25 (q, J=8 Hz, 2H), 4.16 (t, J=4 Hz, 2H), 4.07 (t, J=4 Hz, 2H), 3.87 (t, J=4 Hz, 2H), 3.79-3.73 (m, 6H), 3.60-3.48 (m, 18H), 1.29 (t, J=8 Hz, 3H).

### Example 4: Efficacy Data

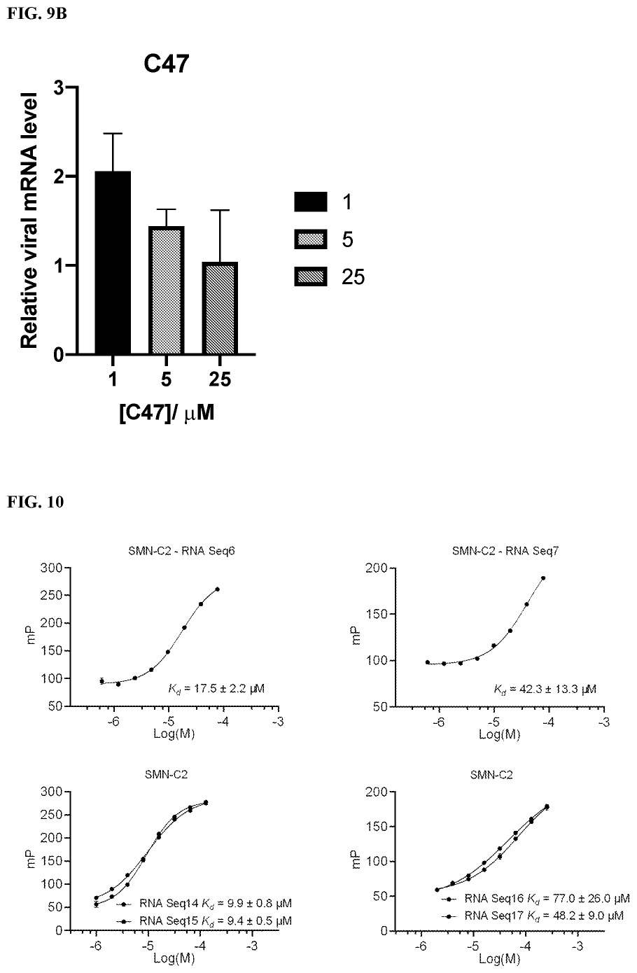

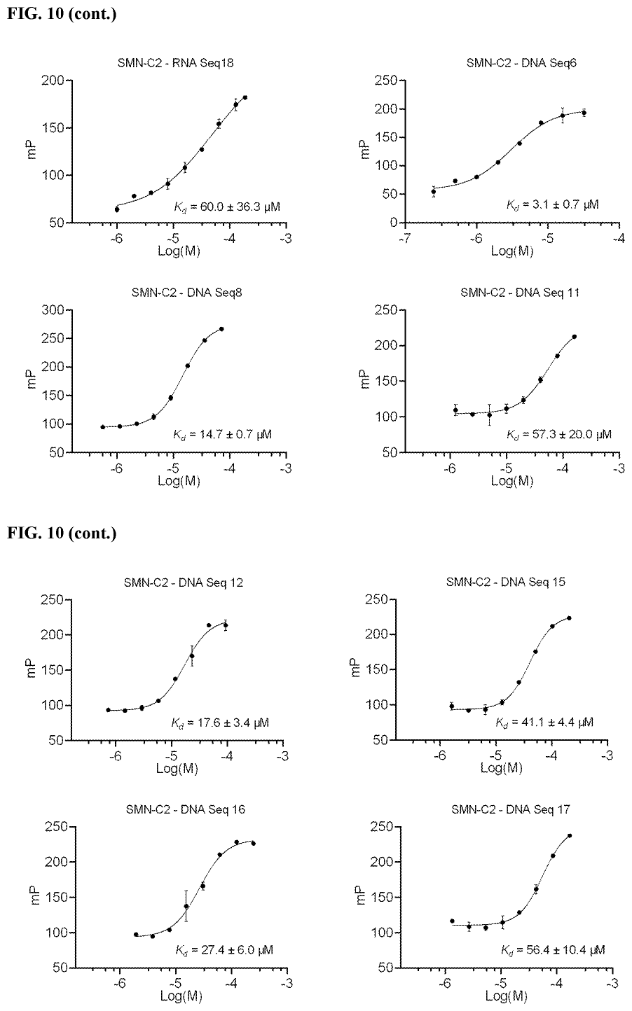

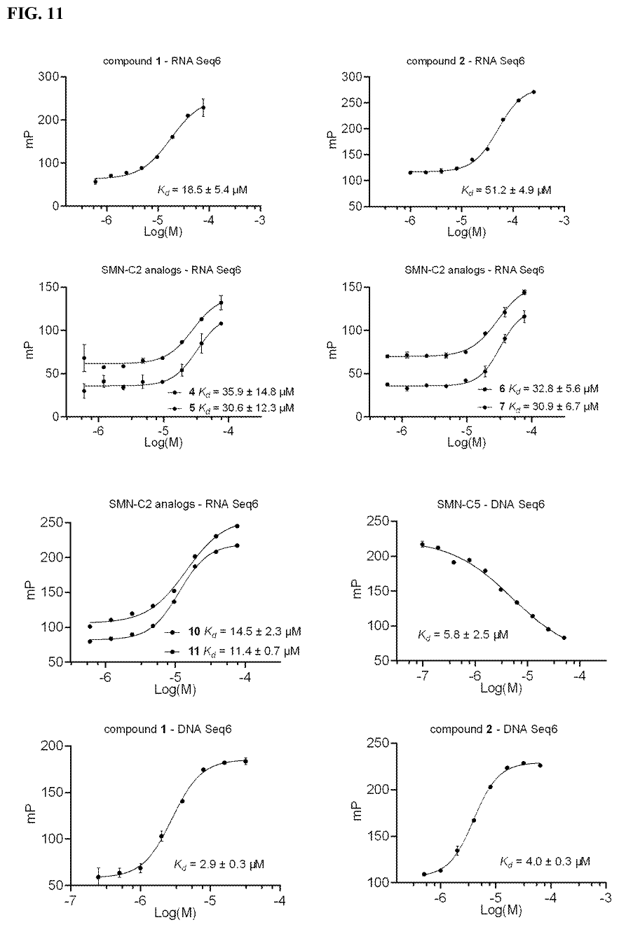

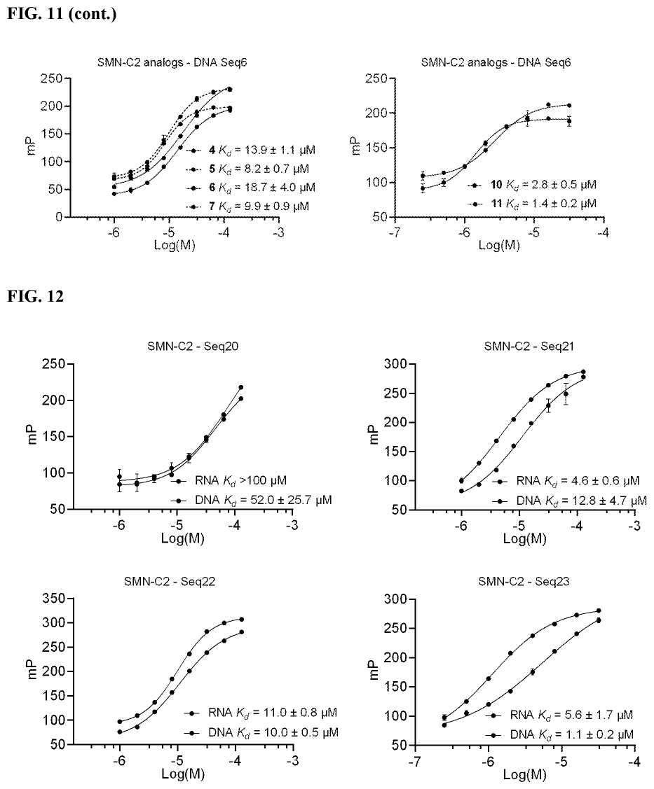

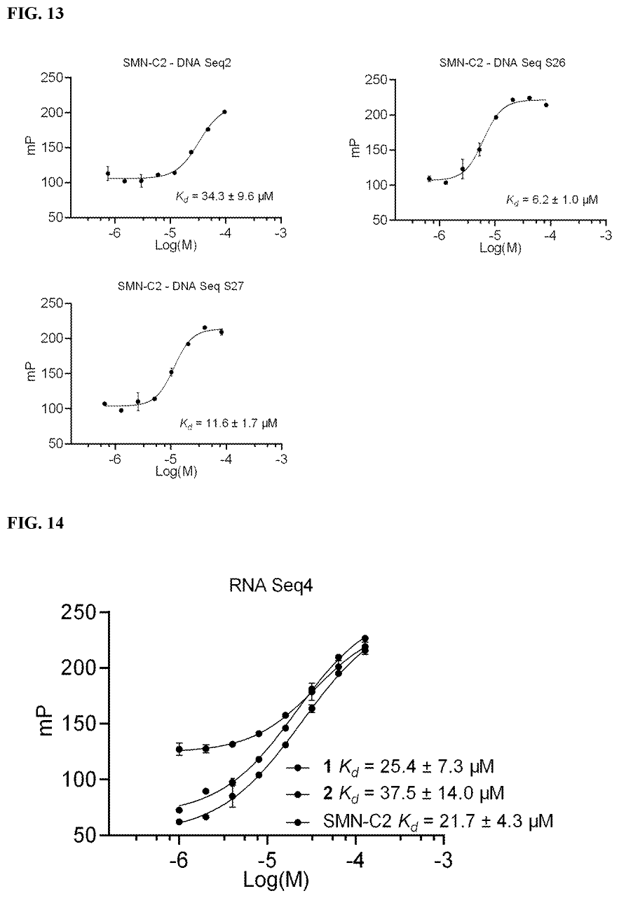

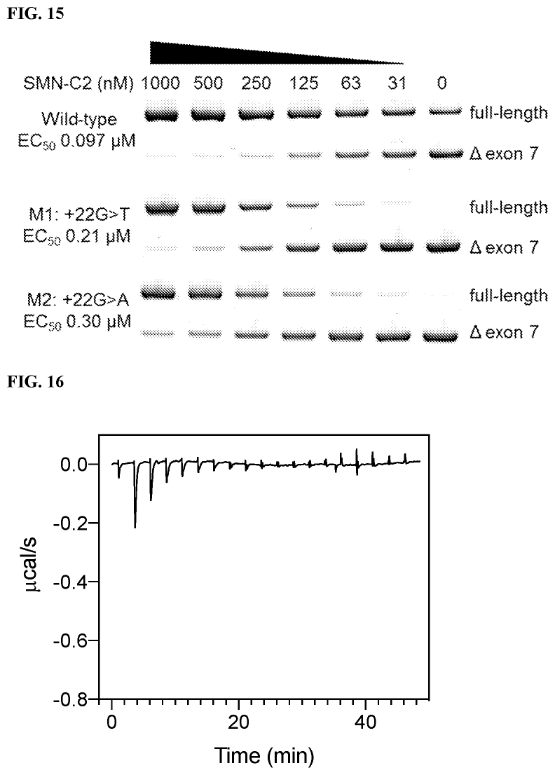

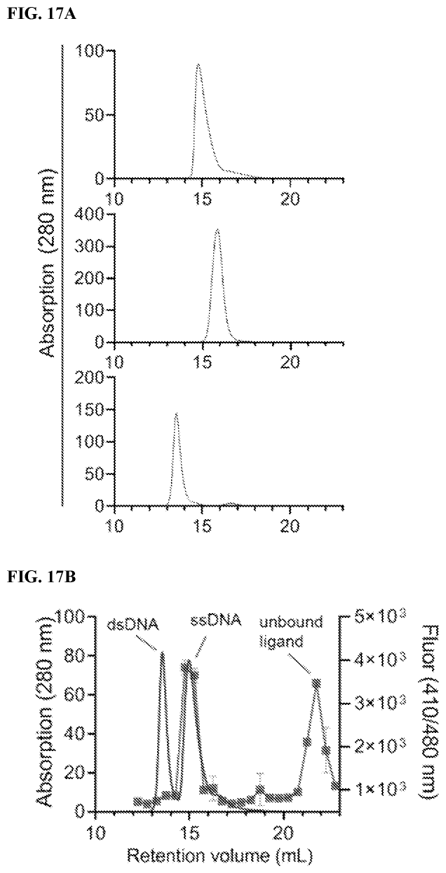

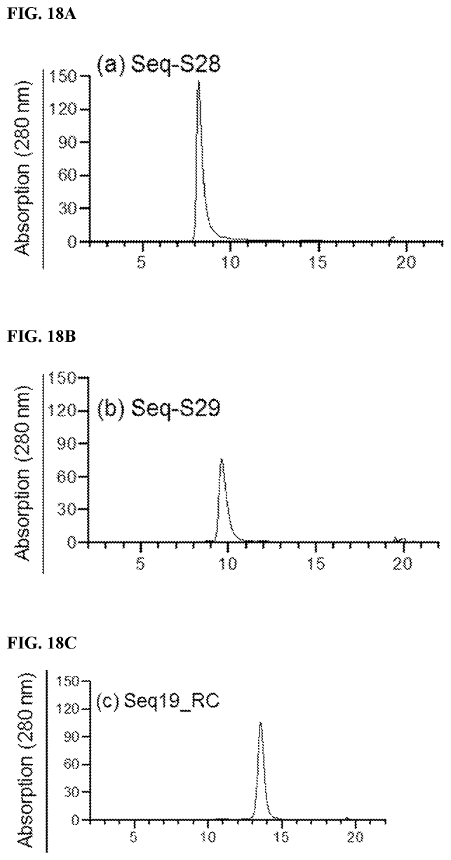

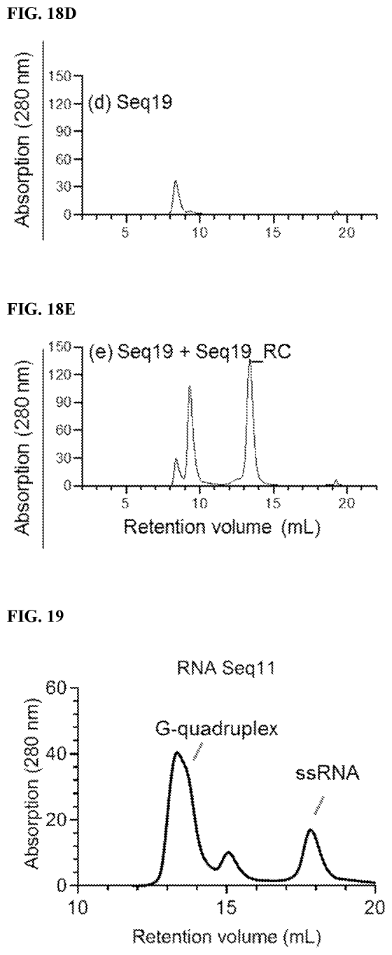

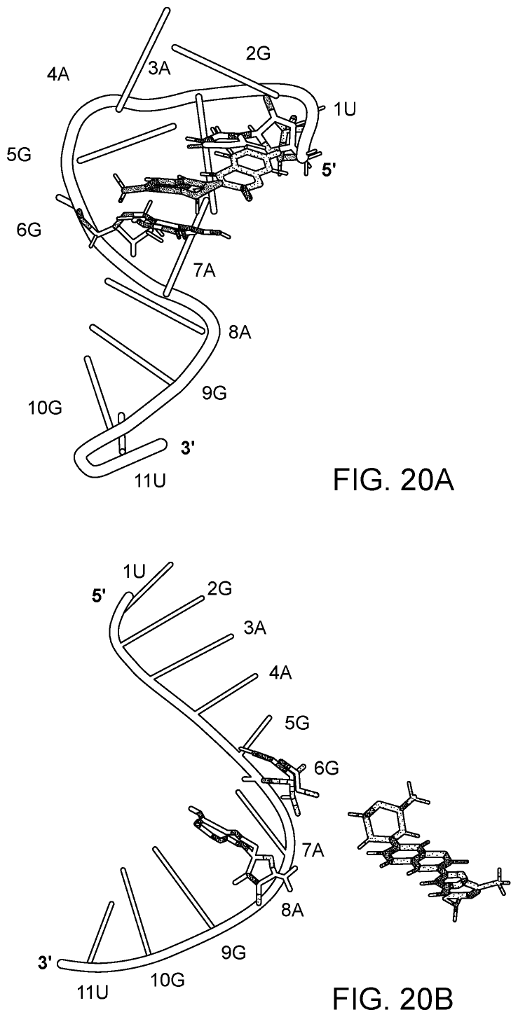

We have tested C47 in a SARS-CoV-2 cell model. Details about the cell model include: a tetracycline-inducible SARS-CoV-2 minigene HEK293 cell-line (ThermoFisher, 293 Flp-In™ T-REx™ system) was constructed. The minigene system contains the full 5′ and 3′ UTRs as well as part of nsp1 in the 5′-end of the viral coding sequence. We replaced the sequence between 5′ and 3′ UTRs with a Gaussia luciferase (FIG. 9A). We have previously validated that the structures in the 5′ UTR in the minigene-transfected cells are almost identical to the one in virus-infected cells². The minigene system can, therefore, be used as a model system for the RNA-targeting approaches.

Brief protocol: SARS-CoV-2 minigene-expressing cells were seeded in a 24-well plate. 5 μg/mL tetracycline and RNA degrader at different concentrations were added to the media (10% FBS in DMEM) simultaneously. After 24 hours, the cells were harvest for real-time (RT) quantitative PCR (qPCR) analysis. The mRNA level of the viral minigene was normalized with an endogenous gene, GAPDH. The primers for the viral gene quantification are listed below: q-hGluc-F: ACCACGGATCTCGATGCTGA; q-hGlucR: TTCATCTTGGGCGTGCACTT.

The RT-qPCR experiment showed that C47, can reduce the RNA level in a dose-dependent manner. In the presence of 25 μM C47, the viral minigene mRNA level reduced ˜50% (FIG. 9B).

### Example 5: SARS-CoV-2—C2NH Preferentially Binds to the SARS-CoV-2 Start Codon

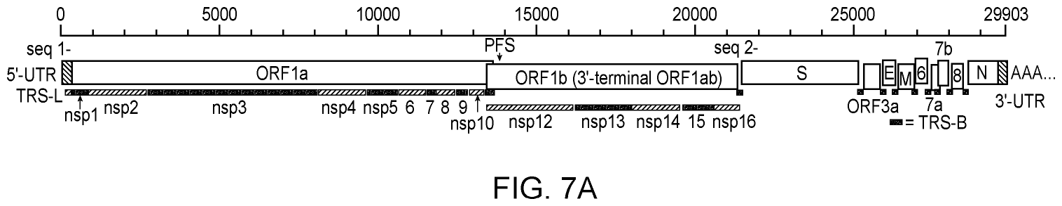

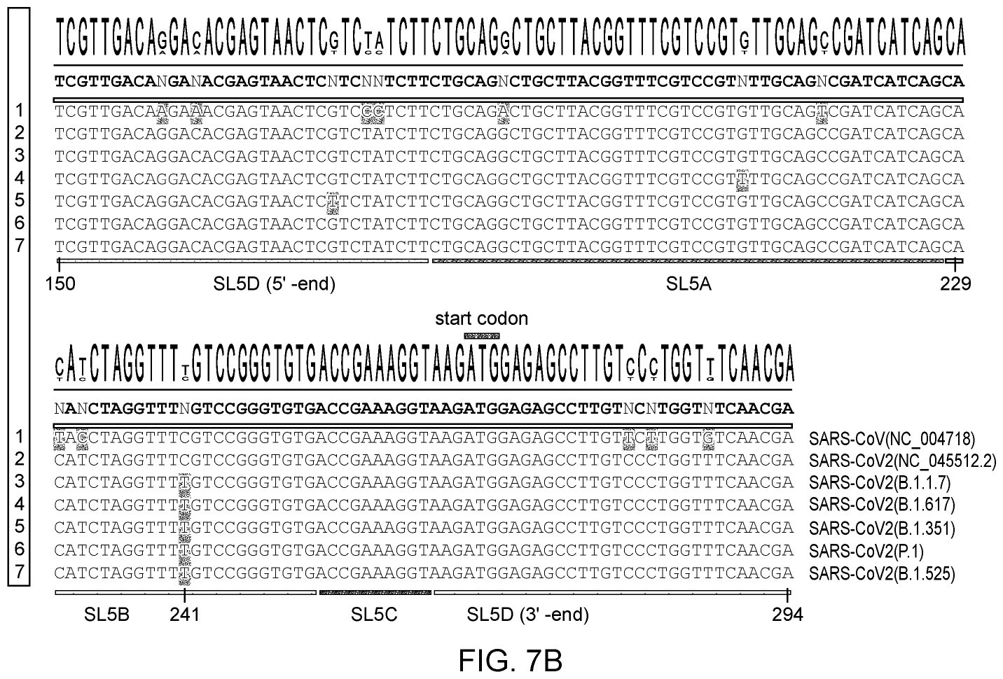

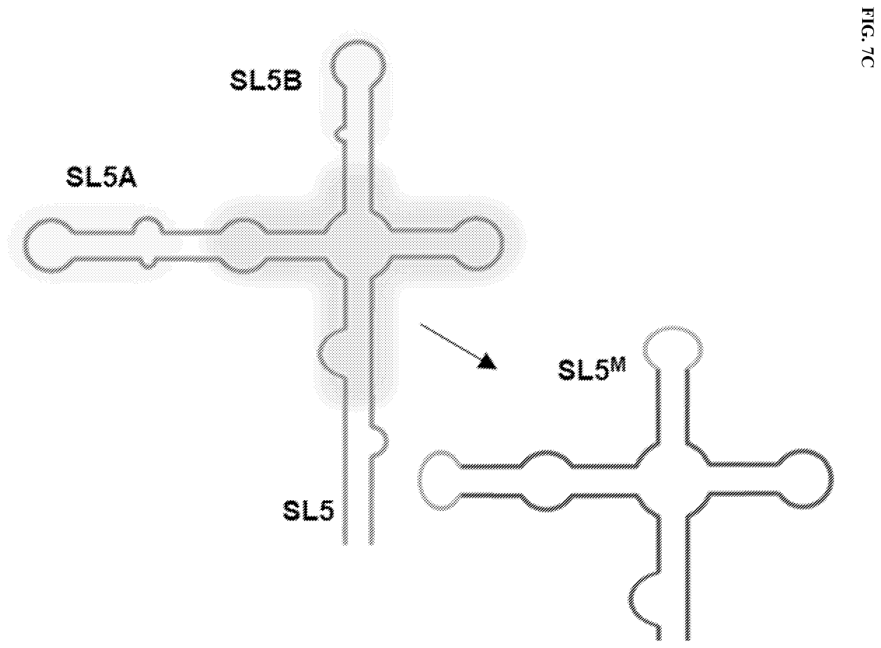

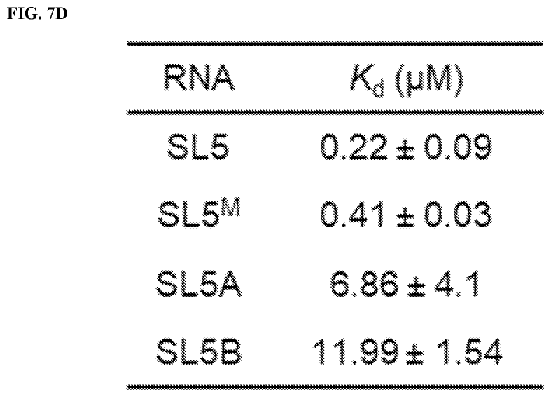

Coronavirus gene assembly and polyproteins. SARS-CoV-2 belongs to the betacoronavirus genus, and is an enveloped ssRNA(+) virus, with a genome length of about 29,903 nucleotides (nts, RefSeq NC_045512)². The viral genome is 5′ capped and 3′ polyadenylated, so that it is recognized and treated as an mRNA by the host cell ribosome. There are 12 open reading frames (ORFs) from 5′ to 3′ (FIG. 7A). The 5′-terminal two-thirds of the genome have two long ORFs, ORF1a and ORF1ab that are translated into two replicase-associated polyprotein (pp) precursors, pp1a and pp1ab. Pp1a is the N-terminal fraction of pp1ab and has an in-fame stop codon at 13,481 nt. Correct translation of C-terminal pp1ab requires a programmed-1 ribosomal frameshift (or programmed frameshift, PFS) that shifts the ORF by −1 nucleotide via a “slippery sequence” to avoid the ORF1a stop codon³. Pp1a and pp1ab are cleaved by viral proteases into 16 nonstructural proteins (nsps), some of which have essential viral functions. For example, nsp12 in pp1ab is required for viral replication, being an RNA-dependent RNA polymerase (RdRp). Nsp12 and other nsps in pp1ab collectively form the replication transcription complex (RTC)⁴. The RTC then promotes replication of the viral genome ssRNA (+), forming a double-stranded (ds) RNA located in ER membrane invaginations. This dsRNA then serves as a template for transcription of further copies of the RNA genome by RTC-mediated transcription from the 5′- to 3′-end. mRNA transcription for each coronavirus structural protein is accomplished through a “discontinuous” mechanism. The RTC binds to the 5′-untranslated region (UTR) leader transcriptional regulatory sequences (TRS-L), and then “hops” onto the body TRS (TRS-B, FIG. 7A) sequence. These TRS-Bs are located at the 5′-end of each structural gene for transcription.

The roles of conserved RNA sequences in viral protein translation. Several conserved sequences have been uncovered in beta coronaviruses^{5,6}. In the proposed study, we will only focus on the essential sequences that have a well-defined function, including the start codon and PFS regulatory sequence. To evaluate the mutation propensity of these two sequences in the SARS-CoV-2 genome, we aligned 3,559 viral sequences uploaded at PubMed (www.ncbi.gov). Our analysis showed that only 1 sequence record has a mutation at the 3′ of the start codon in the region of interest (265-273 nt, FIG. 2A). For the sequence that forms a pseudoknot structure (13,474-13,505 nt+13,538-13,542 nt), only four sites contain single-point mutations in 1-2 records (FIG. 2A). Therefore, we concluded that the target sequences in the proposed study are highly conserved. 1) Start codon function. All viral transcripts share the same start codon sequence, which locates at 3′ of the TBS-L (FIG. 7A). In viral protein translation initiation, the human preinitiation complex, containing the ribosomal 40S subunit, eukaryotic initiation factors (eIFs), and a bound methionyl initiator tRNA in the P-site, is first recruited to the 5′-UTR of the viral RNA in a cap-dependent manner. The ribosomal 40S subunit then “scans” in a 5′ to 3′ direction along the 5′-UTR to locate an AUG start codon (266 nt, FIG. 2A) using the initiator tRNA to start translation elongation⁷. If the viral RNA is cleaved at the start codon region, the transcripts will not be translated.

The mechanism of PFS. PFS is essential for translation of C-terminal pp1ab, which contains almost all components of the RTC. PFS is governed by a highly conserved RNA sequence found in all coronavirus species. This PFS RNA regulatory element contains a slippery sequence (U_UUA_AAC motif) followed by an RNA pseudoknot structure (FIG. 2B)³. Once the ribosome recognizes the pseudoknot, tRNAs in the ribosomal P- and A-sites re-bind to the −1 reading frame, and the ribosome starts to translate within the new reading frame. Without PFS, translation halts at a stop codon (13,481-13,483 nt) within the pseudoknot scaffold. It has been demonstrated that the viral RNA sequence alone (the RNA sequence in FIG. 2B) can recapitulate the PSF activity without a viral protein cofactor in SARS-CoV⁸.

RNA-binding drugs are a validated pharmacological modality as antivirals. RNA viruses use RNA sequences and structures to hijack host cell functions or promote viral life cycle progression, such as the transactivation response (TAR) hairpin, internal ribosomal entry site (IRES), and Rev responsive element (RRE) in HIV-1⁹. The HIV-1 trans-activator protein (Tat) binds to TAR to enhance the transcription of the viral genome. Peptoid inhibitors targeting the TAR-Tat interaction have been shown to inhibit HIV-1 replication in vitro and in vivo¹⁰. In hepatitis C viral (HCV) genome, an IRES in the 5′-UTR recruits the host translation machinery in a 5′-cap-independent manner¹¹. More than six small-molecule IRES-binding scaffolds have been shown to be active in vivo¹²⁻¹⁵ In short, essential RNA sequences have in the past been targeted to interfere with other viruses.

Oligoadenylate synthetase (OAS)-RNase L pathway. In general, OAS-RNase L pathway is activated by double-stranded (ds) RNA, which is produced in RNA virus life cycle. OAS synthesizes a signaling molecule, 2′,5′-linked oligoadenylates (2-5A), that activate RNase L by dimerization. RNase L then cleaves single-stranded (ss) RNA leading to degradation of viral genomes, arrest of protein synthesis, and apoptosis. Coronavirus can inactivate this pathway by destroying the signaling molecule, 2-5A. In a group 2a betacoronavirus, mouse hepatitis virus (MHV), accessory protein ns2 is a 2′,5′-phosphodiesterase (PDE) that cleaves 2-5A, thereby preventing RNase L activation¹⁶. Similarly, MERS also uses PDE activity of a viral gene, ns4b, to enzymatically degrade 2-5A¹⁷. The specific gene that degrades 2-5A has not been fully validated in SARS-CoV-2. The expression of RNase L is not inhibited by SARS-CoV-2 in Calu-3 and A549 cell-lines¹⁸. A small-molecule RNase L dimerizer (i.e. activator) was previously discovered (K_{d}=18 μM to RNase L monomer) and demonstrated a modest antiviral effect as a single agent against human parainfluenza virus in cells¹⁹. Recently, the structure of this RNase L dimerizer was further modify as a recruiter and used as a fragment in RIBOTAC, which precisely degrades a microRNA precursor in cancer cells in vivo, without causing systemic type I interferon upregulation²⁰.

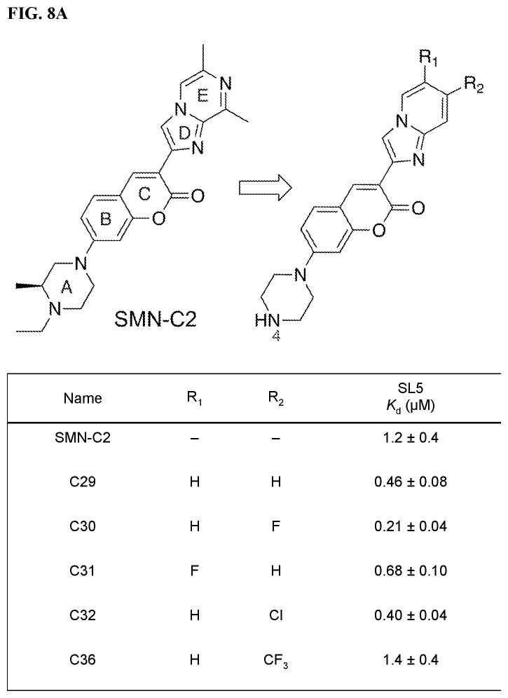

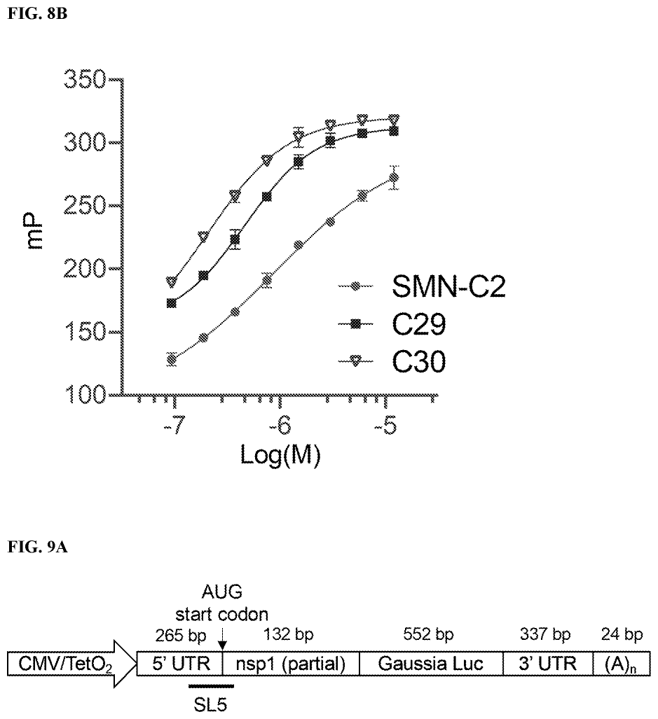

C2NH (FIG. 8A) is a dealkylated analog of a known RNA-binding molecule, SMN-C2²¹, and a close analog of risdiplam (FIG. 8A), an FDA-approved RNA-binding molecule for an unrelated disease, spinal muscular atrophy (SMA, approved on Aug. 7, 2020). The major drug effect of risdiplam is to increase the level of a splice variant of survival of motor neuron (SMN) 2, which should not adversely impact human. Through mutagenesis, we elucidated that C2NH binds to a 9-nt consensus sequence: GRNGGANRG (R=A/G). In the SARS-CoV-2 RNA genome, there are two putative C2NH binding sites (265-273 and 22,018-22,026 nts, FIG. 7A). Using the fluorescence polarization binding assay, we showed that C2NH preferentially binds to seq 1 (GAUGGAGAG, FIG. 8B), which contains the AUG start codon. Seq 1 is similar to the SMN-C2 binding sequence (Seq 3, GAAGGAAGG) in SMN2 gene with a weaker binding affinity. C2NH does not bind to the reverse complement sequence to seq 1 (seq 1-RC, FIG. 8B). We further validated by size-exclusion chromatography that C2NH specifically binds to ssRNA in the presence of dsRNA. We previously determined that 4′-position is a valid conjugation site for RNase L recruiter. The binding affinity of the chimeric molecule and seq 1 remains but the affinity is dampened ˜2-fold (K_{d}=29 μM) likely due to unoptimized conjugation site, spacer, or C2NH fragment.

### Example 6: Biological Data

TABLE 3

| | SMN2 Splicing EC50 | Kd for COVID |
|---|---|---|
| Compound | (nM) | RNA(μM) |
| Risdiplam | | |
| C2 | 63.5 ± 29.6 | 1.6 ± 0.4 |
| C4 | 13.9 ± 2.6 | 1.3 ± 0.3 |
| C6 | | 3.0 ± 0.4 |
| C7 | >8000 | |
| C8 | >8000 | |
| C9 | >8000 | |
| C12 | 180 ± 40 | 2.6 ± 0.8 |
| C13 | 450 ± 180 | |
| C18 | >8000 | |
| C19 | >8000 | |
| C20 | >8000 | |
| C21 | >8000 | |
| C22 | >8000 | |
| C23 | >8000 | |
| C24 | >8000 | |
| C25 | >8000 | |
| C26 | >8000 | |
| C28 | >8000 | 0.47 ± 0.08 |
| C29 | >8000 | 0.46 ± 0.08 |
| C30 | | 0.21 ± 0.04 |
| C31 | | 0.68 ± 0.10 |
| C32 | >8000 | 0.40 ± 0.04 |
| C36 | | 1.4 ± 0.4 |
| C38 | >8000 | |
| C39 | >8000 | |

TABLE 4

| | | CC50 in | CC50 in | CC50 in |
|---|---|---|---|---|
| | Kd for COVID | 293T cells | HepG2 cells | A549 cells |
| Compound | RNA(μM) | (μM) | (μM) | (μM) |
| C30 | 0.21 ± 0.4 | 1.8 | 1 | 2.5 |
| C32 | 0.40 ± 0.4 | 1.5 | 1 | 3.7 |
| C29 | 0.46 ± 0.8 | | | 5.4 |
| C28 | 0.47 ± 0.8 | 1.1 | 1.1 | 7.2 |
| C31 | 0.68 ± 0.1 | | | 4.7 |
| C4 | 1.3 ± 0.3 | 11.5 | 12.2 | >13 |
| C36 | 1.39 ± 0.4 | 4.5 | 2.1 | 6.2 |
| C2 | 1.6 ± 0.4 | >30 | >30 | >30 |
| | (0.82 ± 0.4) | | | |
| C12 | 2.6 ± 0.8 | | | |
| C6 | 3.03 ± 0.4 | 5.6 | 10.4 | 13.1 |
| C13 | | 11.5 | 8.4 | >30 |
| C19 | | >30 | >30 | >30 |
| C22 | | >30 | >30 | >30 |
| C25 | | 14.7 | 4.8 | >30 |
| C26 | | >30 | >30 | >30 |
| C3 | | >30 | >30 | >30 |
| C33 | | | | 11.6 |
| C34 | | | | 3.6 |
| C35 | | | | 3 |
| C37 | | | | 14.1 |
| C38 | | | | 1.8 |
| C39 | | | | 7.9 |

### Example 7

### Minigene SMN2 Splicing Assays in 293T Cells

These in vitro findings were validated in SARS-CoV-2 5′ UTR expressing 293T cells. In this cell model, the SARS-CoV-2 5′ UTR sequence was fused to a CMV promoter-controlled Gaussia luciferase expression cassette. Consistent with the RNase L degradation assay result, the maximum potency of C64 (i.e., RNA reduction level) was significantly better than C65. The activities of C47 and C48 in this cell model are similar, between those of C64 and C65.

The Gaussia luciferase minigenes for SMN2 exon 7 skipping were transfected into 293T cells following the Lipofectamine 2000 protocol (Thermo) in a 6-well plate. After 6 h of incubation, cells were disassociated with 0.5 mL TrypLE (Gibco, #12605036) for 5 min. The trypsinization was stopped by adding 1.5 mL of full growth medium (DIVEM+1000 FBS) to the wells. The cell number was counted using Countess II Automated Cell Counter (Thermo, #AMQAX1000) and were diluted in low serum medium (DMEM+3% FBS). For PAGE, the cells were seeded into a 24-well plate (0.2 million per well) in 0.35 mL medium and incubate for 2 h at 37° C. The cells were then treated with SMN-C2 or nusinersen at various concentrations. In the wells containing nusinersen, 1 μL Endo-Porter delivery reagent (GeneTools, Philomath, OR, USA; ordered through Fisher Scientific, #NC1501848) was added and mixed by gently swirling the plate. The cells were incubated for another 24 h at 37° C. before being harvested by aspirating the medium and adding 300 μL RLT buffer (RNeasy mini kit) in each well. The total RNA was extracted using the RNeasy mini kit (Qiagen, Hilden, Germany #74104) according to the manufacturer's manual. The RNA was reversely transcribed using M-MLV reverse transcriptase (Promega, #M1701) and a poly(dT) primer. The spliced products were amplified by PCR using the primer set, pCI-FW: 5′ GGCTAGAGTACTTAATACGACTCAC, and GLuc-RV: 5′-CAGCGATGCAGATCAGG-GC. The PAGE is performed with 8% TBE gels (180 V, 30 min). The gels were stained with 0.003% SYBR Safe DNA Gel Stain (Thermo, #S33102) in 0.5×TBE buffer for 10 min (for full gel images for FIG. 3).

For luciferase readout, the transfected cells were transferred into a 384-well plate (Greiner #784075) at 15,000 cells per well in 27 μL medium. The compounds were 1:2 serial diluted for 10 concentration points starting at 1 μM (final concentration) and added into the wells in triplicates (3 μL). The plate was incubated for 48 h at 37° C. with 5% CO₂ after the addition of the compounds. The Gaussia luciferase reading agent was prepared by diluting coelenterazine Gaussia luciferase substrate (Thermo #1862575) at 1:500 ratio into the Gaussia luciferase buffer containing 50 mM Tris-C1 pH 7.5, 10 mM MgCl₂, 1 mM DTT, 1 mM ATP, and 0.2% BSA in M-PER Mammalian Protein Extraction Reagent (Thermo #78501). 15 μL of the Gaussia luciferase reading reagent was added to each well and incubated for 5 min at room temperature followed by luminescence measurements (Cytation 5, BioTek). A non-linear regression dose response curve (four parameters) was used for curve-fitting and the EC₅₀ values were determined using GraphPad Prism 8.

The minigene reporter plasmid was constructed by inserting the below sequence between T7 promoter and SV40 poly(A) sequences in pCI vector:

CTAGCCTCGAGATGGCTTTGGGAAGTATGTTAATTTCATGGTACATGAGT
GGCTATCATACTGGCTATTATATGGTAAGTAATCACTCAGCATCTTTTCC
TGACAATTTTTTTGTAGTTATGTGACTTTGTTTTGTAAATTTATAAAATA
CTACTTGCTTCTCTCTTTATATTACTAAAAAATAAAAATAAAAAAATACA
ACTGTCTGAGGCTTAAATTACTCTTGCATTGTCCCTAAGTATAATTTTAG
TTAATTTTAAAAAGCTTTCATGCTATTGTTAGATTATTTTGATTATACAC
TTTTGAATTGAAATTATACTTTTTCTAAATAATGTTTTAATCTCTGATTT
GAAATTGATTGTAGGGAATGGAAAAGATGGGATAATTTTTCATAAATGAA
AAATGAAATTCTTTTTTTTTTTTTTTTTTTTGAGACGGAGTCTTGCTCTG
TTGCCCAGGCTGGAGTGCAATGGCGTGATCTTGGCTCACAGCAAGCTCTG
CCTCCTGGATTCACGCCATTCTCCTGCCTCAACCTCCCAAGTAGCTGGGA
TTAGAGGTCCCCACCACCATGCCTGGCTAATTTTTTGTACTTTCAGTAGA
AACGGGGTTTTGCCATGTTGGCCAGGCTGTTCTCGAACTCCTGAGCTCAG
GTGATCCAACTGTCTCGGCCTCCCAAAGTGCTGGGATTACAGGCGTGAGC
CACTGTGCCTAGCATGAGCCACCACGCCGGCCTAATTTTTAAATTTTTTG
TAGAGACAGGGTCTCATTATGTTGCCCAGGGTGGTGTCAAGCTCCAGGTC
TCAAGTGATCCCCCTACCTCCGCCTCCCAAAGTIGTGGGATTGTAGGCAT
GAGCCACTGCAAGAAAACCTTAACTGCAGCCTAATAATTGTTTTCTTTGG
GATAACTTTTAAAGTACATTAAAAGACTATCAACTTAATTTCTGATCATA
TTTTGTTGAATAAAATAAGTAAAATGTCTTGTGAAACAAAATGCTTTTTA
ACATCCATATAAAGCTATCTATATATAGCTATCTATATCTATATAGCTAT
TTTTTTTAACTTCCTTTATTTTCCTTACAGGGTTTCAGACAAAATCAAAA
AGAAGGAAGGTGCTCACATTCCTTAAATCTAAGGAGTAAGTCTGCCAGCA
TTATGAAAGTGAATCTTACTTTTGTAAAACTTTATGGTTTGTGGAAAACA
AATGTTTTTGAACATTTAAAAAGTTCAGATGTTAGAAAGTTGAAAGGTTA
ATGTAAAACAATCAATATTAAAGAATTTTGATGCCAAAACTATTAGATAA
AAGGTTAATCTACATCCCTACTAGAATTCTCATACTTAACTGGTTGGTTG
TGTGGAAGAAACATACTTTCACAATAAAGAGCTTTAGGATATGATGCCAT
TTTATATCACTAGTAGGCAGACCAGCAGACTTTTTTTTATTGTGATATGG
GATAACCTAGGCATACTGCACTGTACACTCTGACATATGAAGTGCTCTAG
TCAAGTTTAACTGGTGTCCACAGAGGACATGGTTTAACTGGAATTCGTCA
AGCCTCTGGTTCTAATTTCTCATTTGCAGGAAATGCTGGCAAGAGCAGCA
CTAAAGGAGTCAAAGTTCTGTTTGCCCTGATCTGCATCGCTGTGGCCGAG
GCCAAGCCCACCGAGAACAACGAAGACTTCAACATCGTGGCCGTGGCCAG
CAACTTCGCGACCACGGATCTCGATGCTGACCGCGGGAAGTTGCCCGGCA
AGAAGCTGCCGCTGGAGGTGCTCAAAGAGATGGAAGCCAATGCCCGGAAA
GCTGGCTGCACCAGGGGCTGTCTGATCTGCCTGTCCCACATCAAGTGCAC
GCCCAAGATGAAGAAGTTCATCCCAGGACGCTGCCACACCTACGAAGGCG
ACAAAGAGTCCGCACAGGGCGGCATAGGCGAGGCGATCGTCGACATTCCT
GAGATTCCTGGGTTCAAGGACTTGGAGCCCATGGAGCAGTTCATCGCACA
GGTCGATCTGTGTGTGGACTGCACAACTGGCTGCCTCAAAGGGCTTGCCA
ACGTGCAGTGTTCTGACCTGCTCAAGAAGTGGCTGCCGCAACGCTGTGCG
ACCTTTGCCAGCAAGATCCAGGGCCAGGTGGACAAGATCAAGGGGGCCGG
TGGTGACTAACCCGGGCGGCCGCTTCGAGCAGACATGA

Single-point mutations in the plasmid were made in exon 7 (underlined) using Phusion Site-Directed Mutagenesis Kit (Thermo, #F541) with the following target sequences:

WT:
AAAAGAAGGAAGGTGCTC
M1:
AAAATAAGGAAGGTGCTC
M2:
AAAAGGAGGAAGGTGCTC
M3:
AAAAAAAGGAAGGTGCTC
M4:
AAAAGAAGAAAGGTGCTC

### Example 8

### Efficacy of RIBOTAC in Live Virus Infection Assay

Finally, the activity of C64 was tested in SARS-CoV-2 infected cells. The SARS-CoV-2 virus was engineered to include a Nano Luciferase (NLuc) reporter by fusing NLuc onto ORF7 of the SARS-CoV-2 genome45. In this way, the NLuc signal is proportional to the viral protein copy number in cells. A human lung epithelial carcinoma cell line A549 expressing high level of ACE2 was applied as the host cell. The cells were infected with the SARS-CoV-2-NLuc virus at a multiplicity of infection (MOI) of 2.0 at 1 h before the treatment with RIBOTACs C64 for 3 d. C64 showed >95% inhibition at 20 μM. At the same concentration, no major toxicity is observed in A549 cells.

### Example 8: Experimental Conditions

### Isothermal Titration Calorimetry (ITC) Assay

Isothermal calorimetric titrations were carried out on a Malvern Analytical MicroCal PEAQ-ITC at 25° C. The DNA (see above for the calibration procedure) and the SMN-C2 water solutions containing the desired amount of the materials were freeze-dried overnight (Labconco FreeZone 4.5 Liter Benchtop Freeze Dry System) and then re-suspended in appropriate volume of buffer containing 5% DMSO, 100 mM NaCl and 30 mM MES buffer at pH 6.0 (e.g., 350 μL for DNA Seq6, 80 μL for SMN-C2). Each ITC titration had an initial injection of 0.4 μL followed by 18 injections of SMN-C2 (e.g., 250 μM for DNA Seq6 titration) of 2 μL for 4 s at 150 s intervals into the DNA (e.g. Seq6 at 25 μM) in sample cell. The analysis was performed using the instrument's MicroCal PEAQ-ITC Analysis Software (Malvern Analytical). The ITC data was fit using the One Set of Sites mode to calculate the dissociate constant (K_{d}), binding stoichiometry, and the changes in enthalpy and Gibbs free energy. In differential power (DP) trace, the baseline was subtracted for better visualization, which has no impact on calculations.

### Size Exclusion Chromatography

The size exclusion chromatography was performed on a AKTA pure protein purification system (Cytiva, Thousand Oaks, CA, USA) equipped with a Superdex 75 Increase 10/300 GL column (Cytiva #29148721). The samples in 0.1 mL PBS buffer were injected and eluted with PBS buffer (pH. 7.4, 0.5 mL/min, 25 mL). The absorption at 280 nm was monitored in real-time and the chromatography data (A280 over the retention volume) were generated and exported using the instrument analytical software, UNICORN, and replotted with Prism 8. For the annealed mixture of SMN-C2 and DNAs, the eluent was constantly collected in 0.5 mL fractions. The SMN-C2 content in each fraction was subsequently measured using a fluorescence microplate reader (Cytation 5, BioTek, Ex/Em=410/480 nm).

### Surface Plasmon Resonance (SPR) Assay

SPR experiments were performed on a Biacore T200 (GE Healthcare) instrument at 25° C. using streptavidin pre-coated SA sensor chips (GE Healthcare). The running buffer composed of 10 mM HEPES, 100 mM NaCl, 0.05% Tween 20 (w/v), 5 mM EDTA, 0.1% (v/v) DMSO at pH 6.8 was prepared freshly, filtered through the 0.22 m PVDF membrane prior to use. 5′-biotinylated RNA Seq4 was purchased from GenScript and dissolved in nuclease-free water to a concentration of 100 μM. For immobilization of the biotinylated RNA, the sensor chip was firstly conditioned with 3 consecutive 1 min injections of high salt solution (50 mM NaOH, 1M NaCl) at a flow rate of 10 L/min. Next, the biotinylated RNA was diluted 1000× in running buffer (100 nM) and applied over the streptavidin sensor chip surface at a flow rate of 10 μL/min to achieve immobilization level of about 800 RU. Finally, alkyne-PEG-biotin (50 μM in running buffer) was injected (1 min, 10 L/min) to block remaining streptavidin surface binding sites. The kinetics analysis was performed following the BiaControl Software Wizard Kinetics protocol. The small molecules (HCl salt form, 10 mM in water) were diluted in the running buffer to six concentrations (0, 0.1, 1, 5, 10, 20 μM for SMN-C2; 0, 1, 5, 10, 20, 40 μM for SMN-C5; 0, 1, 10, 20, 40, 80 μM for SMN-C3) and titrated over the immobilized RNA Seq4 (contact time: 1 min, flow rate: 30 μL/min). The data analysis was performed using the instrument BiaEvaluation Software. All monitored resonance signals were subtracted with signals from a non-binding reference channel. Kinetic values (K_{d}, kₐ, k_{d}) were calculated using the BiaEvaluation Software Binding Affinity protocol with 1:1 fitting. Figures were plotted using TraceDrawer 1.9.1 (Generic Biophysics Version).

### Gaussian Accelerated Molecular Dynamics (GaMD)

GaMD is an enhanced sampling approach wherein a harmonic boost potential is added to smooth the potential energy surface and reduce energy barriers(1). GaMD provides efficient unconstrained enhanced sampling without the need for predefined collective variables. A brief summary of the method is described here. Consider a system with N atoms at positions r≡{ [symbol] , . . . , [symbol] }. When the system potential V(r) is lower than a reference energy E, the modified potential V*(r) of the system is calculated as:

$${V}^{*} ( \vec{r} ) = V ( \vec{r} ) + Δ V ( \vec{r} ) , \qquad ( 1 )$$

$$Δ V ( \vec{r} ) = \frac{1}{2} {k ( E - V ( \vec{r} ) )}^{2} , V ( \vec{r} ) < E , \qquad ( 2 )$$

where k is the harmonic force constant. The parameters E and k can be determined by applying three principles of enhanced sampling. The reference energy should fall in range as follows:

$${V}_{max} \leq E \leq {V}_{min} + \frac{1}{k} , \qquad ( 3 )$$

where Vₘₐₓ and Vₘᵢₙ are the system maximum and minimum potential energies. To ensure that Eq. (3) is valid, k has to satisfy:

$$k \leq \frac{1}{{V}_{max} - {V}_{min}} .$$

Let us define

$$\leq \frac{1}{{V}_{max} - {V}_{min}} ,$$

then 0<k₀≤1. The standard deviation of ΔV needs to be small enough (i.e., narrow distribution) to ensure precise reweighting using cumulant expansion to the second order: σ_{ΔV}=k(E−V_{avg})σ_{V}<σ₀, where V_{avg} and σ_{V} are the average and standard deviation of AV with as a user-specified upper limit (e.g. 10 k_{B}T) for accurate reweighting. When E is set to the lower bound E=Vₘₐₓ, k₀ can be calculated as:

$${k}_{0} = min ( 1. , {k}_{0}^{′} ) = min ( 1. , \frac{{σ}_{0}}{{σ}_{V}} \cdot \frac{{V}_{max} - {V}_{min}}{{V}_{max} - {V}_{avg}} ) \qquad ( 4 )$$

Alternatively, when the threshold energy E is set to its upper bound

$$E = {V}_{min} + \frac{1}{k} , {k}_{0}$$

is set to:

$${k}_{0} = {k}_{0}^{″} ≡ ( 1 - \frac{{σ}_{0}}{{σ}_{V}} ) \cdot \frac{{V}_{max} - {V}_{min}}{{V}_{avg} - {V}_{min}} \qquad ( 5 )$$

If k₀″ is calculated between 0 and 1. Otherwise, k₀″ is calculated using Eq. (4).

The original GaMD method provides schemes to add only the total potential boost ΔV_{P}, only dihedral potential boost ΔV_{D}, or the dual potential boost (both ΔV_{P} and ΔV_{D}). Dual-boost GaMD provides higher acceleration than the other two types of simulations. The simulation parameters comprise of the threshold energy E for applying boost potential and effective harmonic force constants, k₀ₚ and k_{0D} and for total and dihedral potential boost.

### Energetic Reweighting of GaMD Simulations

The GaMD simulations can be reweighted to calculate the original potential mean force (PMF) free energy profiles. The probability distribution along a reaction coordinate is written as p*(A). Given the boost potential ΔV(r) of each frame, p*(A) can be reweighted to recover the canonical ensemble distribution p(A), as:

$$p ( {A}_{j} ) = {p}^{*} ( {A}_{j} ) \frac{{〈 {e}^{βΔ V ( r )} 〉}_{j}}{{\sum }_{i = 1}^{M} {〈 {p}^{*} ( {A}_{i} ) {e}^{βΔ V ( r )} 〉}_{i}} , j = 1 , … , M , \qquad ( 6 )$$

where M is the number of bins, β=k_{B}T and [symbol] e^{βΔV(r)} [symbol] ⱼ is the ensemble-averaged Boltzmann factor of ΔV(r) for simulation frames found in the j^{th} bin. The ensemble-averaged reweighting factor can be approximated using cumulant expansion:

$${〈 {e}^{βΔ V ( r )} 〉}_{j} = exp \{ {\sum }_{k = 1}^{∞} \frac{{β}^{k}}{k !} {C}_{k} \} , \qquad ( 7 )$$

where first two cumulants are given by

$${C}_{1} = 〈 Δ V 〉 , \qquad ( 8 )$$

$${C}_{2} = 〈 Δ {V}^{2} 〉 - {〈 Δ V 〉}^{2} = {σ}_{V}^{2} .$$

The boost potential derived from GaMD simulations usually follows near-Gaussian distribution. Cumulant expansion to the second order thus provides a good approximation for computing the reweighting factor(1, 2). The reweighted free energy F(A)=−k_{B}T ln p(A) is calculated as:

$$F ( A ) = {F}^{*} ( A ) - {\sum }_{k = 1}^{2} \frac{{β}^{k}}{k !} {C}_{k} + {F}_{c} , \qquad ( 9 )$$

where F*(A)=−k_{B}T ln p*(A) is the modified free energy obtained from GaMD simulation and F, is a constant.

### Chemistry

Reagents and solvents were purchased from commercial sources (Fisher, Sigma-Aldrich and Combi-Blocks) and used as received. Reactions were tracked by TLC (Silica gel 60 F254, Merck) and Waters ACQUITY UPLC-MS system (ACQUITY UPLC H Class Plus in tandem with Qda Mass Detector). Intermediates and products were purified by a Teledyne ISCO Combi-Flash system using prepacked SiO₂ cartridges. NMR spectra were acquired on a Bruker AV400 instrument (400 MHz for ¹H NMR, 100 MHz for ¹³C NMR) or Bruker AV500 instrument (500 MHz for ¹H NMR, 125 MHz for ¹³C NMR). Data were recorded as follows: chemical shift (6) in ppm, coupling constant (J) in Hz, multiplicity (s=singlet, d=doublet, t=triplet, m=multiplet or overlap of nonequivalent resonances, brs=broad singlet). ¹³C shifts were obtained with ¹H decoupling. MS-ESI spectra were recorded on Waters Qda Mass Detector. HPLC was performed on Waters ACQUITY UPLC H Class Plus system using Waters BEH C18 (2.1 mm×50 mm, 1.7 m) column and peak detection at 254 nm with UV.

SMN-C2, SMN-C3, SMN-C5, compound 1 were synthesized following procedures reported in literature(3).

### (S)-tert-butyl(2-(4-(3-(6,8-dimethylimidazo[1,2-a]pyrazin-2-yl)-2-oxo-2H-chromen-7-yl)-2-methylpiperazin-1-yl)ethyl)carbamate. (Compound 2)

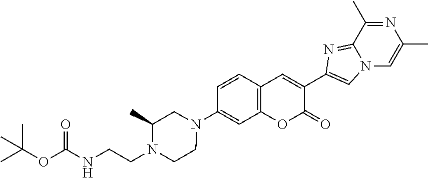 [SMILES: Cc1cn2cc(-c3cc4ccc(N5CCN(CCNC(=O)OC(C)(C)C)[C@@H](C)C5)cc4oc3=O)nc2c(C)n1]

To a solution of compound 1 (10 mg, 0.025 mmol) and tert-butyl (2-bromoethyl) carbamate (11 mg, 0.05 mmol) in acetonitrile was added Cs₂CO₃ (24 mg, 0.075 mmol), the reaction mixture was heated to 70° C. and stirred overnight. TLC and LC-MS showed completion of reaction. The reaction mixture was cooled to room temperature, filtered and concentrated under vacuum. The residue was purified by column chromatography (0-5% CH₃OH in CH₂Cl₂) to afford 6 mg yellow solid. (yield, 43.9%). ¹H NMR (400 MHz, CDCl₃): δ 8.74 (s, 1H), 8.45 (s, 1H), 7.77 (s, 1H), 7.51 (d, J=8.8 Hz, 1H), 6.86 (dd, J=8.8, 2.4 Hz, 1H), 6.75 (d, J=2.4 Hz, 1H), 4.96 (brs, 1H), 3.59 (t, J=12 Hz, 2H), 3.31-3.20 (m, 3H), 3.03-2.92 (m, 6H), 2.67 (brs, 1H), 2.49-2.33 (m, 5H), 1.48 (s, 9H), 1.17 (d, J=6.4 Hz, 3H); ¹³C NMR (100 MHz, CDCl₃): δ 160.7, 156.0, 155.5, 153.4, 151.2, 140.1, 139.3, 138.9, 137.1, 129.3, 115.4, 114.4, 113.6, 111.9, 111.1, 100.3, 79.3, 54.8, 54.3, 52.4, 49.9, 47.5, 28.5, 20.8, 20.5, 15.8. MS-ESI (m/z) [M+H]⁺ 533.29.

### General Procedures for Compounds 3 to 9

Step 1: Synthesis of Coumarin intermediate: A mixture of substituted phenyl acetic acid (1 eq), substituted 2-hydroxylbenzaldehyde (1 eq), triethylamine and acetic anhydride (1:5) was heated to 100° C. for 1 h in a Biotage Initiator⁺ microwave reactor. TLC and LC-MS showed completion of reaction. The mixture was cooled to room temperature, poured into 10 mL ice water and extracted with ethyl acetate. Organic layer was washed with brine, dried with anhydrous sodium sulphate and removed under vacuum. Acetonitrile was added to the residue and the resulted precipitate was filtered and washed with acetonitrile to afford a white or yellow solid which was used without further purification.

Step 2: Synthesis of final product: To a solution of Coumarin intermediate (1 eq) and 1-methylpiperazine (2 eq) in DMSO was added K₂CO₃ (3 eq). The reaction mixture was heated to 120° C. and stirred for 2 h. TLC and LC-MS showed completion of reaction. The mixture was cooled to room temperature, poured into ice water and extracted with ethyl acetate. Organic layer was washed with brine, dried with anhydrous sodium sulphate and removed under vacuum. The residue was purified by column chromatography (0-5% CH₃OH in CH₂Cl₂) to afford product as yellow solid.

### 3-(2-chlorophenyl)-7-(4-methylpiperazin-1-yl)-2H-chromen-2-one (compound 3)

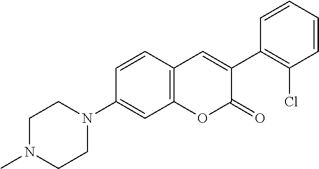 [SMILES: CN1CCN(c2ccc3cc(-c4ccccc4Cl)c(=O)oc3c2)CC1]

Following General Procedures, from 2-(2-chlorophenyl)acetic acid (85 mg, 0.5 mmol) and 4-fluoro-2-hydroxybenzaldehyde (70 mg, 0.5 mmol), 10 mg of compound 3 was obtained as yellow solid (two steps yield, 9.6%). ¹H NMR (400 MHz, CDCl₃): δ 7.62 (s, 1H), 7.48-7.46 (m, 1H), 7.42-7.40 (m, 1H), 7.35 (d, J=8.8 Hz, 1H), 7.32-7.30 (m, 2H), 6.84 (dd, J=8.8, 2.4 Hz, 1H), 6.77 (d, J=2.4 Hz, 1H), 3.40 (t, J=5.2 Hz, 4H), 2.60 (t, J=5.2 Hz, 4H), 2.38 (s, 3H); ¹³C NMR (100 MHz, CDCl₃): δ 160.7, 156.2, 153.8, 142.9, 134.6, 134.0, 131.8, 130.0, 129.7, 128.9, 126.9, 122.0, 111.9, 110.9, 101.1, 54.7, 47.6, 46.2. MS-ESI (m/z) [M+H]⁺ 355.13.

### 3-(3-chlorophenyl)-7-(4-methylpiperazin-1-yl)-2H-chromen-2-one (compound 4)

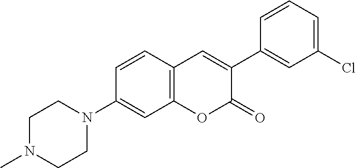 [SMILES: CN1CCN(c2ccc3cc(-c4cccc(Cl)c4)c(=O)oc3c2)CC1]

Following General Procedures, from 2-(3-chlorophenyl)acetic acid (85 mg, 0.5 mmol) and 4-fluoro-2-hydroxybenzaldehyde (70 mg, 0.5 mmol), 13 mg of compound 4 was obtained as yellow solid (two steps yield, 12.5%). ¹H NMR (500 MHz, CDCl₃): δ 7.72 (s, 1H), 7.69 (t, J=2.0 Hz, 1H), 7.60 (dt, J=7.0, 2.0 Hz, 1H), 7.38-7.33 (m, 3H), 6.84 (dd, J=9.0, 2.5 Hz, 1H), 6.75 (d, J=2.5 Hz, 1H), 3.40 (t, J=5.0 Hz, 4H), 2.60 (brs, 4H), 2.39 (s, 3H); ¹³C NMR (125 MHz, CDCl₃): δ 161.1, 155.9, 153.7, 140.8, 137.3, 134.4, 129.7, 129.0, 128.4, 128.3, 126.7, 121.8, 112.0, 111.3, 100.8, 54.7, 47.5, 46.1. MS-ESI (m/z) [M+H]⁺ 355.15.

### 3-(4-chlorophenyl)-7-(4-methylpiperazin-1-yl)-2H-chromen-2-one (compound 5)

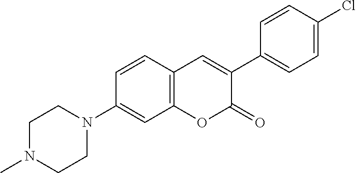 [SMILES: CN1CCN(c2ccc3cc(-c4ccc(Cl)cc4)c(=O)oc3c2)CC1]

Following General Procedures, from 2-(4-chlorophenyl)acetic acid (85 mg, 0.5 mmol) and 4-fluoro-2-hydroxybenzaldehyde (70 mg, 0.5 mmol), 11 mg of compound 5 was obtained as yellow solid (two steps yield, 10.5%). ¹H NMR (500 MHz, CDCl₃): δ 7.71 (s, 1H), 7.65 (dt, J=9.0, 2.5 Hz, 2H), 7.40-7.37 (m, 3H), 6.84 (dd, J=8.5, 2.5 Hz, 1H), 6.75 (d, J=2.5 Hz, 1H), 3.43 (brs, 4H), 2.65 (brs, 4H), 2.42 (s, 3H); ¹³C NMR (125 MHz, CDCl₃): δ 161.2, 155.8, 153.5, 140.2, 134.2, 134.0, 129.8, 128.9, 128.7, 122.2, 112.0, 111.4, 100.9, 54.6, 47.4, 46.0. MS-ESI (m/z) [M+H]⁺ 355.14.

### 3-(3-chlorophenyl)-5-fluoro-7-(4-methylpiperazin-1-yl)-2H-chromen-2-one (Compound 6)

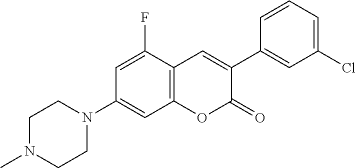 [SMILES: CN1CCN(c2cc(F)c3cc(-c4cccc(Cl)c4)c(=O)oc3c2)CC1]

Following General Procedures, from 2-(3-chlorophenyl)acetic acid (51 mg, 0.3 mmol) and 2,4-difluoro-6-hydroxybenzaldehyde (50 mg, 0.3 mmol), 9 mg of compound 6 was obtained as yellow solid (two steps yield, 13.7%). ¹H NMR (500 MHz, CDCl₃): δ 7.90 (s, 1H), 7.69 (t, J=2.0 Hz, 1H), 7.59 (dt, J=6.5, 2.0 Hz, 1H), 7.37-7.34 (m, 2H), 6.54-6.51 (m, 2H), 3.37 (t, J=5.0 Hz, 4H), 2.56 (t, J=5.0 Hz, 4H), 2.37 (s, 3H); ¹³C NMR (125 MHz, CDCl₃): δ 161.0, 160.5, 159.0, 156.0, 153.7, 137.0, 134.5, 133.9, 129.8, 128.5, 126.7, 121.5, 101.3, 97.5, 96.4, 54.6, 47.4, 46.2. MS-ESI (m/z) [M+H]⁺ 373.15.

### 3-(4-chlorophenyl)-5-fluoro-7-(4-methylpiperazin-1-yl)-2H-chromen-2-one (Compound 7)

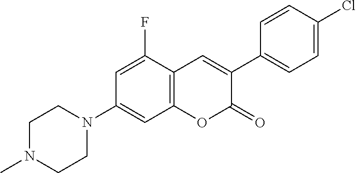 [SMILES: CN1CCN(c2cc(F)c3cc(-c4ccc(Cl)cc4)c(=O)oc3c2)CC1]

Following General Procedures, from 2-(4-chlorophenyl)acetic acid (51 mg, 0.3 mmol) and 2,4-difluoro-6-hydroxybenzaldehyde (50 mg, 0.3 mmol), 10 mg of compound 7 was obtained as yellow solid (two steps yield, 15.2%). ¹H NMR (500 MHz, CDCl₃): δ 7.87 (s, 1H), 7.64 (dt, J=9.0, 2.5 Hz, 2H), 7.39 (dt, J=9.0, 2.5 Hz, 2H), 6.53-6.50 (m, 1H), 3.37 (t, J=5.0 Hz, 4H), 2.56 (t, J=5.0 Hz, 4H), 2.37 (s, 3H); ¹³C NMR (125 MHz, CDCl₃): δ 160.9, 160.7, 159.0, 155.9, 153.6, 134.4, 133.7, 133.3, 129.8, 128.7, 121.7, 101.4, 97.5, 96.4, 54.6, 47.4, 46.2. MS-ESI (m/z) [M+H]⁺ 373.13.

### 3-(3-chlorophenyl)-4-methyl-7-(4-methylpiperazin-1-yl)-2H-chromen-2-one (Compound 8)

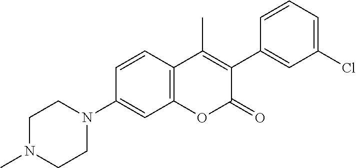 [SMILES: Cc1c(-c2cccc(Cl)c2)c(=O)oc2cc(N3CCN(C)CC3)ccc12]

Following General Procedures, from 2-(3-chlorophenyl)acetic acid (85 mg, 1 mmol) and 1-(4-fluoro-2-hydroxyphenyl)ethanone (77 mg, 1 mmol), 12 mg of compound 8 was obtained as light yellow solid (two steps yield, 11.1%). ¹H NMR (500 MHz, CDCl₃): δ 7.51 (d, J=9.0 Hz, 1H), 7.39-7.34 (m, 2H), 7.30 (t, J=2.0 Hz, 1H), 7.19 (dt, J=6.5, 2.0 Hz, 1H), 6.86 (dd, J=9.0, 2.5 Hz, 1H), 6.76 (d, J=2.5 Hz, 1H), 3.41 (t, J=5.0 Hz, 4H), 2.64 (brs, 4H), 2.41 (s, 3H), 2.25 (s, 3H); ¹³C NMR (125 MHz, CDCl₃): δ 161.5, 154.7, 153.5, 148.6, 136.9, 134.3, 130.6, 129.7, 128.8, 128.2, 126.1, 121.9, 112.0, 111.7, 101.3, 54.7, 47.5, 46.1, 16.6. MS-ESI (m/z) [M+H]⁺ 369.17.

### 3-(4-chlorophenyl)-4-methyl-7-(4-methylpiperazin-1-yl)-2H-chromen-2-one (Compound 9)

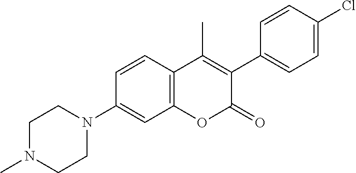 [SMILES: Cc1c(-c2ccc(Cl)cc2)c(=O)oc2cc(N3CCN(C)CC3)ccc12]

Following General Procedures, from 2-(4-chlorophenyl)acetic acid (85 mg, 0.5 mmol) and 1-(4-fluoro-2-hydroxyphenyl)ethanone (77 mg, 0.5 mmol), 11 mg of compound 9 was obtained as light yellow solid (two steps yield, 10.1%). ¹H NMR (500 MHz, CDCl₃): δ 7.50 (d, J=8.5 Hz, 1H), 7.41 (dt, J=8.5, 2.0 Hz, 2H), 7.24 (dt, J=8.5, 2.0 Hz, 2H), 6.86 (dd, J=9.0, 2.5 Hz, 1H), 6.76 (d, J=2.0 Hz, 1H), 3.39 (t, J=5.0 Hz, 4H), 2.61 (brs, 4H), 2.39 (s, 3H), 2.25 (s, 3H); ¹³C NMR (125 MHz, CDCl₃): δ 161.6, 154.7, 153.4, 148.4, 134.0, 133.5, 132.0, 128.7, 126.1, 122.0, 112.1, 111.7, 101.3, 54.7, 47.6, 46.1, 16.5. MS-ESI (m/z) [M+H]⁺ 369.15.

### 3-(imidazo[1,2-a]pyridin-2-yl)-7-(piperazin-1-yl)-2H-chromen-2-one. (compound 10)

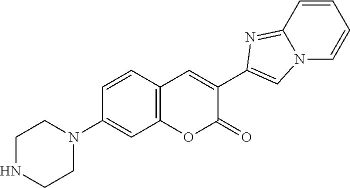 [SMILES: O=c1oc2cc(N3CCNCC3)ccc2cc1-c1cn2ccccc2n1]

A mixture of 3-(2-bromoacetyl)-7-fluoro-2H-chromen-2-one (50 mg, 0.17 mmol) and 2-aminopyridine (16 mg, 0.17 mmol) in acetonitrile was heated to 120° C. for 20 min in a Biotage Initiator⁺ microwave reactor. TLC and LC-MS showed completion of reaction. The reaction mixture was filtered and the filter cake was washed with acetonitrile to afford a yellow solid which was used in next step without further purification.

To a solution of the above intermediate (40 mg, 0.14 mmol) and piperazine (24 mg, 0.28 mmol) in DMSO was added K₂CO₃ (58 mg, 0.42 mmol). The reaction mixture was heated to 120° C. and stirred for 2 h. The mixture was cooled to room temperature and then poured into ice water. The precipitation was filtered, washed and dried. The residue was purified by column chromatography (0-10% CH₃OH in CH₂Cl₂) to afford 32 mg of compound 10 as yellow solid (two steps yield, 52.7%). ¹H NMR (500 MHz, DMSO-_{d6}): δ 8.72 (s, 1H), 8.62 (d, J=7.0 Hz, 1H), 8.52 (s, 1H), 7.69 (d, J=9.0 Hz, 1H), 7.54 (d, J=9.0 Hz, 1H), 7.29-7.25 (m, 1H), 7.01 (dd, J=9.0, 2.5 Hz, 1H), 6.90-6.85 (m, 2H), 3.29 (t, J=5 Hz, 4H), 2.82 (t, J=5 Hz, 4H); ¹³C NMR (125 MHz, DMSO-_{d6}): δ 159.7, 155.0, 153.8, 144.2, 138.6, 138.5, 129.6, 127.3, 125.6, 116.1, 114.6, 112.2, 111.7, 110.2, 99.2, 48.0, 45.4. MS-ESI (m/z) [M+H]⁺ 347.14.

### 3-(6-methoxyimidazo[1,2-a]pyrazin-2-yl)-7-(piperazin-1-yl)-2H-chromen-2-one. (Compound 11)

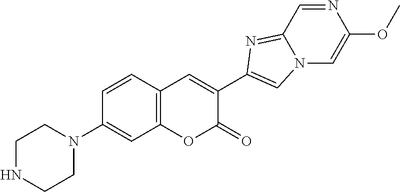 [SMILES: COc1cn2cc(-c3cc4ccc(N5CCNCC5)cc4oc3=O)nc2cn1]

Following procedures for compound 10, from 3-(2-bromoacetyl)-7-fluoro-2H-chromen-2-one (30 mg, 0.1 mmol) and 5-methoxypyrazin-2-amine (13 mg, 0.1 mmol), 8 mg of compound 11 was obtained as yellow solid (two steps yield, 20.1%). ¹H NMR (500 MHz, DMSO-_{d6}): δ 8.82 (s, 1H), 8.76 (s, 1H), 8.60 (s, 1H), 8.31 (d, J=1.5 Hz, 1H), 7.70 (d, J=9.0 Hz, 1H), 7.02 (dd, J=9.0, 2.5 Hz, 1H), 6.85 (d, J=2 Hz, 1H), 3.87 (s, 3H), 3.32 (t, J=5 Hz, 4H), 2.83 (t, J=5 Hz, 4H); ¹³C NMR (125 MHz, DMSO-_{d6}): δ 159.7, 155.3, 154.0, 152.7, 141.6, 139.7, 138.8, 138.3, 129.9, 113.7, 111.7, 109.9, 102.4, 99.1, 55.0, 47.7, 45.3. MS-ESI (m/z) [M+H]⁺ 378.17.

TABLE 5
Summary of GaMD simulations performed on the DNA and
RNA Seq6 in the presence of the drug compound 1.

| | | | | ΔV_{avg}^{[b]} | σ_{ΔV}^{[c]} |
|---|---|---|---|---|---|
| System | Nₐₜₒₘₛ^{[a]} | ID | Length(ns) | (kcal/mol) | (kcal/mol) |
| DNA: compound 1 | 38,192 | Sim 1 | 500 | 11.54 | 3.40 |
| | | Sim 2 | 500 | 10.85 | 3.29 |
| | | Sim 3 | 500 | 11.36 | 3.33 |
| | | Sim 4 | 500 | 11.12 | 3.34 |
| | | Sim 5 | 500 | 11.50 | 3.45 |
| RNA: compound 1 | 38,113 | Sim 1 | 500 | 12.39 | 3.44 |
| | | Sim 2 | 500 | 12.03 | 3.43 |
| | | Sim 3 | 500 | 11.89 | 3.40 |
| | | Sim 4 | 500 | 11.99 | 3.39 |
| | | Sim 5 | 500 | 12.20 | 3.43 |

^{[a]}Nₐₜₒₘₛ: number of atoms in the system
^{[b]}ΔV_{avg}: average of the GaMD boost potential
^{[c]}σ_{ΔV}: standard deviation of the GaMD boost potential

TABLE 6
Binding affinities between SMN-C2 and
9- and 10-nt DNAs containing the putative
binding sequences in the fluorescence
polarization (FP) assay.

| Seq# | Sequence | K_{d} (μM) |
|---|---|---|
| Seq2 | d(GAAGGAAGG) | 34.3 ± 9.6 |
| S26 | d(TGAAGGAAGG) | 6.2 ± 1.0 |
| S27 | d(AGAAGGAAGG) | 11.6 ± 1.7 |

TABLE 7
Binding affinities between SMN-C2 and 18-nt
DNA sequences determined by the fluorescence
polarization (FP) assay.

| | DNA sequence | K_{d} | Fit |
|---|---|---|---|
| Seq# | (18 nt) | (μM)^{[a]} | SeqCS^{[b]} |
| S1 | AAAAGAAGGAAGGTGCTC | 15.3 ± 0.6 | Yes |
| S2 | AAAAGAAGGAAGGAGCAC | 20.2 ± 1.7 | Yes |
| S3 | AAAAGAAGGAAGGTGATA | 21.7 ± 2.7 | Yes |
| S4 | AAAAAGAAGAAGGTGCTC | >50 | No |
| S5 | AAAAGAAUGAAGGTGCTC | 42.3 ± 4.2 | No |
| S6 | AAAAGAAGGAGGGTGCTC | 33.3 ± 2.3 | Yes |
| S7 | AATAGAAGGAAGGTGCTC | 17.3 ± 1.7 | Yes |
| S8 | AAATGAAGGAAGGTGCTC | 12.4 ± 0.4 | Yes |
| S9 | AAAATAAGGAAGGTGCTC | 56.2 ± 13.9 | No |
| S10 | AAAAGTAGGAAGGTGCTC | 26.4 ± 2.2 | No |
| S11 | AAAAGATGGAAGGTGCTC | 26.3 ± 1.2 | No |
| S12 | AAAAGAAGTAAGGTGCTC | 37.4 ± 6.7 | No |
| S13 | AAAAGAAGGTAGGTGCTC | 26.4 ± 2.2 | No |
| S14 | AAAAGAAGGATGGTGCTC | 19.5 ± 2.3 | No |
| S15 | AAAAGAAGGAATGTGCTC | 31.4 ± 4 | No |
| S16 | AAAAGAAGGAAGTTGCTC | 48.9 ± 12.2 | No |
| S17 | AAAGGAAGGAAGGTGCTC | 16.5 ± 1.1 | Yes |
| S18 | AAAAGGAGGAAGGTGCTC | >50 | No |
| S19 | AAAAGAGGGAAGGTGCTC | 21.0 ± 1.1 | Yes |
| S21 | AAAAAAAGGAAGGTGCTC | >50 | No |
| S22 | AAAAGAAAGAAGGTGCTC | 44.7 ± 7.2 | No |
| S23 | AAAAGAAGAAAGGTGCTC | >50 | No |
| S24 | AAAAGAAGGAAAGTGCTC | 36.8 ± 11.4 | No |
| S25 | AAAAGAAGGAAGATGCTC | 48 ± 7.8 | No |

^{[a]}The binding affinity was determined using florescence polarization assay (see Methods). The assay was duplicated, and the range was calculated with 95% confidence interval using Prism software after curve-fitting (Sigmoidal, 4 parameters).
^{[b]}The consensus sequence SeqCS = NGARGGARGGN (R = A or G; N = A, T, G, or C). All sequences with a K_{d} < 25 μM concur with the consensus sequence (SeqSC) except S6 in the table (K_{d} = 33.3 ± 2.3 μM). All sequences that do not match the SeqSC has K_{d} > 25 μM.

TABLE 8
Binding affinities between RNA Seq4 and
SMN-C2, compound 1, and compound 2 in
fluorescence polarization (FP) assay.

| Compound | Seq4 | K_{d} (μM) |
|---|---|---|
| SMN-C2 | AAGAAGGAAGGUGC | 21.7 ± 4.3 |
| 1 | AAGAAGGAAGGUGC | 25.4 ± 7.3 |
| 2 | AAGAAGGAAGGUGC | 37.5 ± 14.0 |

### Example 9: Live Virus Assay

The activity of additional compounds of the present technology will be tested in SARS-CoV-2-infected HAE-ALI cultures. The HAE infection assay will use polarized primary human bronchial epithelial cells that mimic the native microenvironment in the lung. Briefly, the cells will be plated onto the Transwell inserts (0.33 cm²/0.4 μM pore size, Coster #3470) at an air-liquid interface (ALI) for 4 weeks. In the basolateral chamber, the airway epithelium will be incubated with the RIBOTACs at 24 h before, concurrent, or 24 h after apical infection of SARS-CoV-2, to evaluate the specific timing of the inhibitory effect in viral replication. SARS-CoV-2 (isolate USA-WA1/2020), which will be propagated in HAE-ALI to ensure no mutations/deletions (<1%) gain in the viral genome during the propagation by RNA-seq. Viruses will be inoculated by incubation of the diluted virus in 100 μl of D-PBS at an MOI of 0.2 (high), 0.02 (medium), and 0.002 (low) in the apical chamber.

### REFERENCES CITED IN EXAMPLE 1

1. Lunn, M. R. and Wang, C. H. (2008) Spinal muscular atrophy. Lancet, 371, 2120-33.
2. Lorson, C. L., Rindt, H. and Shababi, M. (2010) Spinal muscular atrophy: mechanisms and therapeutic strategies. Hum. Mol. Genet., 19, R111-8.
3. D'Amico, A., Mercuri, E., Tiziano, F. D. and Bertini, E. (2011) Spinal muscular atrophy. Orphanet J. Rare Dis., 6, 71.
4. Lorson, C. L., Hahnen, E., Androphy, E. J. and Wirth, B. (1999) A single nucleotide in the SMN gene regulates splicing and is responsible for spinal muscular atrophy. Proc. Natl. Acad Sci. U.S.A., 96, 6307-11.
5. Kashima, T., Rao, N., David, C. J. and Manley, J. L. (2007) hnRNP A1 functions with specificity in repression of SMN2 exon 7 splicing. Hum. Mol. Genet., 16, 3149-59.
6. Cartegni, L. and Krainer, A. R. (2002) Disruption of an SF2/ASF-dependent exonic splicing enhancer in SMN2 causes spinal muscular atrophy in the absence of SMN1. Nat. Genet., 30, 377-84.
7. Finkel, R. S., Mercuri, E., Darras, B. T., Connolly, A. M., Kuntz, N. L., Kirschner, J., Chiriboga, C. A., Saito, K., Servais, L., Tizzano, E., et al. (2017) Nusinersen versus Sham Control in Infantile-Onset Spinal Muscular Atrophy. N. Engl. J. Med, 377, 1723-1732.
8. Ratni, H., Ebeling, M., Baird, J., Bendels, S., Bylund, J., Chen, K. S., Denk, N., Feng, Z., Green, L., Guerard, M., et al. (2018) Discovery of Risdiplam, a Selective Survival of Motor Neuron-2 (SMN2) Gene Splicing Modifier for the Treatment of Spinal Muscular Atrophy (SMA). J. Med Chem., 61, 6501-6517.
9. Hua, Y., Vickers, T. A., Okunola, H. L., Bennett, C. F. and Krainer, A. R. (2008) Antisense masking of an hnRNP A1/A2 intronic splicing silencer corrects SMN2 splicing in transgenic mice. Am. J. Hum. Genet., 82, 834-48.
10. Hua, Y., Vickers, T. A., Baker, B. F., Bennett, C. F. and Krainer, A. R. (2007) Enhancement of SMN2 exon 7 inclusion by antisense oligonucleotides targeting the exon. PLoS Biol., 5, e73.
11. Ratni, H., Karp, G. M., Weetall, M., Naryshkin, N. A., Paushkin, S. V, Chen, K. S., McCarthy, K. D., Qi, H., Turpoff, A., Woll, M. G., et al. (2016) Specific Correction of Alternative Survival Motor Neuron 2 Splicing by Small Molecules: Discovery of a Potential Novel Medicine To Treat Spinal Muscular Atrophy. J. Med Chem., 59, 6086-100.
12. Naryshkin, N., Weetall, M., Dakka, A., Narasimhan, J., Zhao, X., Feng, Z., Ling, K. K. Y., Karp, G. M., Qi, H., Woll, M. G., et al. (2014) Motor neuron disease. SMN2 splicing modifiers improve motor function and longevity in mice with spinal muscular atrophy. Science, 345, 688-93.
13. Sturm, S., Günther, A., Jaber, B., Jordan, P., Al Kotbi, N., Parkar, N., Cleary, Y., Frances, N., Bergauer, T., Heinig, K., et al. (2019) A phase 1 healthy male volunteer single escalating dose study of the pharmacokinetics and pharmacodynamics of risdiplam (RG7916, RO7034067), a SMN2 splicing modifier. Br. J. Clin. Pharmacol., 85, 181-193.
14. Wang, J., Schultz, P. G. and Johnson, K. A. (2018) Mechanistic studies of a small-molecule modulator of SMN2 splicing. Proc. Natl. Acad. Sci., 115, E4604-E4612.
15. Sivaramakrishnan, M., McCarthy, K. D., Campagne, S., Huber, S., Meier, S., Augustin, A., Heckel, T., Meistermann, H., Hug, M. N., Birrer, P., et al. (2017) Binding to SMN2 pre-mRNA-protein complex elicits specificity for small molecule splicing modifiers. Nat. Commun., 8.
16. Singh, N. N., Singh, R. N. and Androphy, E. J. (2007) Modulating role of RNA structure in alternative splicing of a critical exon in the spinal muscular atrophy genes. Nucleic Acids Res., 35, 371-89.
17. Campagne, S., Boigner, S., Rüdisser, S., Moursy, A., Gillioz, L., Knörlein, A., Hall, J., Ratni, H., Cléry, A. and Allain, F. H.-T. (2019) Structural basis of a small molecule targeting RNA for a specific splicing correction. Nat. Chem. Biol., 15, 1191-1198.
18. Singh, R. N., Ottesen, E. W. and Singh, N. N. (2020) The First Orally Deliverable Small Molecule for the Treatment of Spinal Muscular Atrophy. Neurosci. Insights, 15.
19. Brodersen, D. E., Clemons, W. M., Carter, A. P., Morgan-Warren, R. J., Wimberly, B. T. and Ramakrishnan, V. (2000) The structural basis for the action of the antibiotics tetracycline, pactamycin, and hygromycin B, on the 30S ribosomal subunit. Cell, 103, 1143-1154.
20. Anokhina, M. M., Barta, A., Nierhaus, K. H., Spiridonova, V. A. and Kopylov, A. M. (2004) Mapping of the second tetracycline binding site on the ribosomal small subunit of E. coli. Nucleic Acids Res., 32, 2594-2597.
21. Pioletti, M., Schlünzen, F., Harms, J., Zarivach, R., Gluhmann, M., Avila, H., Bashan, A., Bartels, H., Auerbach, T., Jacobi, C., et al. (2001) Crystal structures of complexes of the small ribosomal subunit with tetracycline, edeine and IF3. EMBO J., 20, 1829-1839.
22. Cheatham, T. E. and Kollman, P. A. (2000) Molecular dynamics simulation of nucleic acids. Annu. Rev. Phys. Chem., 51, 435-471.
23. Sponer, J., Bussi, G., Krepl, M., Banas, P., Bottaro, S., Cunha, R. A., Gil-Ley, A., Pinamonti, G., Poblete, S., Jurecka, P., et al. (2018) RNA structural dynamics as captured by molecular simulations: A comprehensive overview. Chem. Rev., 118, 4177-4338.
24. Stelzer, A. C., Frank, A. T., Kratz, J. D., Swanson, M. D., Gonzalez-Hernandez, M. J., Lee, J., Andricioaei, I., Markovitz, D. M. and Al-Hashimi, H. M. (2011) Discovery of selective bioactive small molecules by targeting an RNA dynamic ensemble. Nat. Chem. Biol., 7, 553-559.
25. Di Leva, F. S., Novellino, E., Cavalli, A., Parrinello, M. and Limongelli, V. (2014) Mechanistic insight into ligand binding to G-quadruplex DNA. Nucleic Acids Res., 42, 5447-5455.
26. Moraca, F., Amato, J., Ortuso, F., Artese, A., Pagano, B., Novellino, E., Alcaro, S., Parrinello, M. and Limongelli, V. (2017) Ligand binding to telomeric G-quadruplex DNA investigated by funnel-metadynamics simulations. Proc. Natl. Acad. Sci. U.S.A., 114, E2136-E2145.
27. Levintov, L. and Vashisth, H. (2020) Ligand Recognition in Viral RNA Necessitates Rare Conformational Transitions. J. Phys. Chem. Lett., 11, 5426-5432.
28. Wang, J., Arantes, P. R., Bhattarai, A., Hsu, R. V., Pawnikar, S., Huang, Y. M., Palermo, G. and Miao, Y. (2021) Gaussian accelerated molecular dynamics: Principles and applications. WIREs Comput. Mol. Sci., 10.1002/wcms.1521.
29. Miao, Y., Feher, V. A. and McCammon, J. A. (2015) Gaussian Accelerated Molecular Dynamics: Unconstrained Enhanced Sampling and Free Energy Calculation. J. Chem. Theory Comput., 11, 3584-3595.
30. Pawnikar, S. and Miao, Y. (2020) Pathway and mechanism of drug binding to chemokine receptors revealed by accelerated molecular simulations. Future Med. Chem., 12, 1213-1225.
31. Bhattarai, A. and Miao, Y. (2018) Gaussian accelerated molecular dynamics for elucidation of drug pathways. Expert Opin. Drug Discov., 13, 1055-1065.
32. Case, D. A., Belfon, K., Ben-Shalom, I. Y., Brozell, S. R., Cerutti, D. S., Cheatham, T. E., Cruzeiro, V. W. D., Darden, T. A., Duke, R. E., Giambasu, G., et al. (2020) AMBER 2020, University of California, San Francisco.
33. Jo, S., Cheng, X., Lee, J., Kim, S., Park, S. J., Patel, D. S., Beaven, A. H., Lee, K. I., Rui, H., Park, S., et al. (2017) CHARMM-GUI 10 years for biomolecular modeling and simulation. J. Comput. Chem., 38, 1114-1124.
34. Case, D. A., Ben-Shalom, I. Y., Brozell, S. R., Cerutti, D. S., Cheatham, T. E., Cruzeiro, V. W. D., Darden, T. A., Duke, R. E., Ghoreishi, D., Gilson, M. K., et al. (2018) AMBER 2018, University of California, San Francisco.
35. Humphrey, W., Dalke, A. and Schulten, K. (1996) VMD: Visual molecular dynamics. J Mol. Graph., 14, 33-38.
36. Roe, D. R. and Cheatham, T. E. (2013) PTRAJ and CPPTRAJ: Software for processing and analysis of molecular dynamics trajectory data. J. Chem. Theory Comput., 9, 3084-3095.
37. Miao, Y., Sinko, W., Pierce, L., Bucher, D., Walker, R. C. and McCammon, J. A. (2014) Improved reweighting of accelerated molecular dynamics simulations for free energy calculation. J. Chem. Theory Comput., 10, 2677-2689.
38. Sakamoto, T., Morita, S., Tabata, K., Nakamura, K. and Kawai, G. (2002) Solution Structure of a SRP19 Binding Domain in Human SRP RNA. J. Biochem., 132, 177-182.
39. Okada, K., Takahashi, M., Sakamoto, T., Kawai, G., Nakamura, K. and Kanai, A. (2006) Solution Structure of a GAAG Tetraloop in Helix 6 of SRP RNA from Pyrococcus furiosus. Nucleosides, Nucleotides and Nucleic Acids, 25, 383-395.
40. Mayer, M. and Meyer, B. (1999) Characterization of Ligand Binding by Saturation Transfer Difference NMR Spectroscopy. Angew. Chemie Int. Ed., 38, 1784-1788.
41. Sigel, A., Operschall, B. P. and Sigel, H. (2014) Comparison of the π-stacking properties of purine versus pyrimidine residues. Some generalizations regarding selectivity. JBIC J. Biol. Inorg. Chem., 19, 691-703.
42. Cléry, A., Jayne, S., Benderska, N., Dominguez, C., Stamm, S. and Allain, F. H. T. (2011) Molecular basis of purine-rich RNA recognition by the human SR-like protein Tra2-β1. Nat. Struct. Mol. Biol., 18, 443-451.
43. Warner, K. D., Hajdin, C. E. and Weeks, K. M. (2018) Principles for targeting RNA with drug-like small molecules. Nat. Rev. Drug Discov., 17, 547-558.
44. Eubanks, C. S., Forte, J. E., Kapral, G. J. and Hargrove, A. E. (2017) Small Molecule-Based Pattern Recognition To Classify RNA Structure. J. Am. Chem. Soc., 139, 409-416.
45. Hargrove, A. E. (2020) Small molecule-RNA targeting: starting with the fundamentals. Chem. Commun., 56, 14744-14756.
46. Velagapudi, S. P., Luo, Y., Tran, T., Haniff, H. S., Nakai, Y., Fallahi, M., Martinez, G. J., Childs-Disney, J. L. and Disney, M. D. (2017) Defining RNA-Small Molecule Affinity Landscapes Enables Design of a Small Molecule Inhibitor of an Oncogenic Noncoding RNA. ACS Cent. Sci., 3, 205-216.
47. Howe, J. A., Wang, H., Fischmann, T. O., Balibar, C. J., Xiao, L., Galgoci, A. M., Malinverni, J. C., Mayhood, T., Villafania, A., Nahvi, A., et al. (2015) Selective small-molecule inhibition of an RNA structural element. Nature, 526, 672-677.
48. Velagapudi, S. P., Gallo, S. M. and Disney, M. D. (2014) Sequence-based design of bioactive small molecules that target precursor microRNAs. Nat. Chem. Biol., 10, 291-297.
49. Disney, M. D., Winkelsas, A. M., Velagapudi, S. P., Southern, M., Fallahi, M. and Childs-Disney, J. L. (2016) Inforna 2.0: A Platform for the Sequence-Based Design of Small Molecules Targeting Structured RNAs. ACS Chem. Biol., 11, 1720-1728.
50. Morgan, B. S., Forte, J. E., Culver, R. N., Zhang, Y. and Hargrove, A. E. (2017) Discovery of Key Physicochemical, Structural, and Spatial Properties of RNA-Targeted Bioactive Ligands. Angew. Chemie—Int. Ed., 56, 13498-13502.
51. Correll, C. C. and Swinger, K. (2003) Common and distinctive features of GNRA tetraloops based on a GUAA tetraloop structure at 1.4 Å resolution. RNA, 9, 355-363.
52. Okada, K., Takahashi, M., Sakamoto, T., Kawai, G., Nakamura, K. and Kanai, A. (2006) Solution structure of a GAAG tetraloop in helix 6 of SRP RNA from Pyrococcus furiosus. Nucleosides, Nucleotides and Nucleic Acids, 25, 383-395.
53. Nakano, M., Moody, E. M., Liang, J. and Bevilacqua, P. C. (2002) Selection for Thermodynamically Stable DNA Tetraloops Using Temperature Gradient Gel Electrophoresis Reveals Four Motifs: d(cGNNAg), d(cGNABg), d(cCNNGg), and d(gCNNGc) t. Biochemistry, 41, 14281-14292.
54. Mondal, S. I., Sen, S., Hazra, A. and Patwari, G. N. (2017) 71-Stacked Dimers of Fluorophenylacetylenes: Role of Dipole Moment. J. Phys. Chem. A, 121, 3383-3391.
55. Han, X., Zhao, L., Xiang, W., Qin, C., Miao, B., Xu, T., Wang, M., Yang, C.-Y., Chinnaswamy, K., Stuckey, J., et al. (2019) Discovery of Highly Potent and Efficient PROTAC Degraders of Androgen Receptor (AR) by Employing Weak Binding Affinity VHL E3 Ligase Ligands. J. Med. Chem., 62, 11218-11231.
56. Palacino, J., Swalley, S. E., Song, C., Cheung, A. K., Shu, L., Zhang, X., Van Hoosear, M., Shin, Y., Chin, D. N., Keller, C. G., et al. (2015) SMN2 splice modulators enhance U1-pre-mRNA association and rescue SMA mice. Nat. Chem. Biol., 11, 511-517.
57. Ando, S., Suzuki, S., Okubo, S., Ohuchi, K., Takahashi, K., Nakamura, S., Shimazawa, M., Fuji, K. and Hara, H. (2020) Discovery of a CNS penetrant small molecule SMN2 splicing modulator with improved tolerability for spinal muscular atrophy. Sci. Rep., 10, 1-16.
58. Su, Z., Zhang, Y., Gendron, T. F., Bauer, P. O., Chew, J., Yang, W. Y., Fostvedt, E., Jansen-West, K., Belzil, V. V., Desaro, P., et al. (2014) Discovery of a Biomarker and Lead Small Molecules to Target r(GGGGCC)-Associated Defects in c9FTD/ALS. Neuron, 83, 1043-1050.

### REFERENCES CITED IN EXAMPLE 2

1. Woll, M. G. et al. Discovery and Optimization of Small Molecule Splicing Modifiers of Survival Motor Neuron 2 as a Treatment for Spinal Muscular Atrophy. J. Med. Chem. 59, 6070-6085 (2016). DOI: 10.1021/acs.jmedchem.6b00460.
2. Ratni, H. et al. Specific Correction of Alternative Survival Motor Neuron 2 Splicing by Small Molecules: Discovery of a Potential Novel Medicine To Treat Spinal Muscular Atrophy. J. Med. Chem. 59, 6086-100 (2016). DOI: 10.1021/acs.jmedchem.6b00459.

### REFERENCES CITED IN EXAMPLE 4

1. Haniff, H. S. et al. Targeting the SARS-CoV-2 RNA Genome with Small Molecule Binders and Ribonuclease Targeting Chimera (RIBOTAC) Degraders. ACS Cent. Sci. 6, 1713-1721 (2020). DOI: 10.1021/acscentsci.0c00984.
2. Zhao, J., Qiu, J., Aryal, S., Hackett, J. L. & Wang, J. The RNA Architecture of the SARS-CoV-2 3′-Untranslated Region. Viruses 12, 1473 (2020). DOI: 10.3390/v12121473. PMCID: PMC7766253. References cited in Example 5
1. Beigel, J. H. et al. Remdesivir for the Treatment of Covid-19 Preliminary Report. N. Engl. J. Med. NEJMoa2007764 (2020) doi:10.1056/NEJMoa2007764.
2. Wu, F. et al. A new coronavirus associated with human respiratory disease in China. Nature 579, 265-269 (2020).
3. Hagemeijer, M., Rottier, P. & Haan, C. Biogenesis and Dynamics of the Coronavirus Replicative Structures. Viruses 4, 3245-3269 (2012).
4. Fung, T. S. & Liu, D. X. Human Coronavirus: Host-Pathogen Interaction. Annu. Rev. Microbiol. 73, 529-557 (2019).
5. Chan, J. F.-W. et al. Genomic characterization of the 2019 novel human-pathogenic coronavirus isolated from a patient with atypical pneumonia after visiting Wuhan. Emerg. Microbes Infect. 9, 221-236 (2020).
6. Rangan, R., Zheludev, I. N. & Das, R. RNA genome conservation and secondary structure in SARS-CoV-2 and SARS-related viruses: a first look. RNA rna.076141.120 (2020) doi:10.1261/rna.076141.120.
7. Hinnebusch, A. G. The Scanning Mechanism of Eukaryotic Translation Initiation. Annu. Rev. Biochem. 83, 779-812 (2014).
8. Baranov, P. V. et al. Programmed ribosomal frameshifting in decoding the SARS-CoV genome. Virology 332, 498-510 (2005).
9. Connelly, C. M., Moon, M. H. & Schneekloth, J. S. The Emerging Role of RNA as a Therapeutic Target for Small Molecules. Cell Chem. Biol. 23, 1077-1090 (2016).
10. Hamy, F. et al. An inhibitor of the Tat/TAR RNA interaction that effectively suppresses HIV-1 replication. Proc. Natl. Acad Sci. 94, 3548-3553 (1997).
11. Dibrov, S. M. et al. Hepatitis C Virus Translation Inhibitors Targeting the Internal Ribosomal Entry Site. J. Med Chem. 57, 1694-1707 (2014).
12. Wang, W. et al. Hepatitis C viral IRES inhibition by phenazine and phenazine-like molecules. Bioorg. Med Chem. Lett. 10, 1151-4 (2000).
13. Seth, P. P. et al. SAR by MS: Discovery of a New Class of RNA-Binding Small Molecules for the Hepatitis C Virus: Internal Ribosome Entry Site IIA Subdomain. J. Med Chem. 48, 7099-7102 (2005).
14. Gooding, K. B. et al. High throughput screening of library compounds against an oligonucleotide substructure of an RNA target. J. Am. Soc. Mass Spectrom. 15, 884-892 (2004).
15. Baugh, C., Wang, S., Li, B., Appleman, J. R. & Thompson, P. A. SCAN—a high-throughput assay for detecting small molecule binding to RNA targets. J. Biomol. Screen. 14, 219-29 (2009).
16. Silverman, R. H. & Weiss, S. R. Viral phosphodiesterases that antagonize double-stranded RNA signaling to RNase L by degrading 2-5A. J. Interferon Cytokine Res. 34, 455-63 (2014).
17. Thornbrough, J. M. et al. Middle East Respiratory Syndrome Coronavirus NS4b Protein Inhibits Host RNase L Activation. MBio 7, e00258 (2016).
18. Blanco-Melo, D. et al. Imbalanced Host Response to SARS-CoV-2 Drives Development of COVID-19. Cell 181, 1036-1045.e9 (2020).
19. Thakur, C. S. et al. Small-molecule activators of RNase L with broad-spectrum antiviral activity. Proc. Natl. Acad. Sci. U.S.A. 104, 9585-90 (2007).
20. Costales, M. G. et al. Small-molecule targeted recruitment of a nuclease to cleave an oncogenic RNA in a mouse model of metastatic cancer. Proc. Natl. Acad. Sci. 117, 2406-2411 (2020).

### REFERENCES CITED IN EXAMPLE 7

1. Miao, Y., Feher, V. A. and McCammon, J. A. (2015) Gaussian Accelerated Molecular Dynamics: Unconstrained Enhanced Sampling and Free Energy Calculation. J. Chem. Theory Comput., 11, 3584-3595.
2. Miao, Y., Sinko, W., Pierce, L., Bucher, D., Walker, R. C. and McCammon, J. A. (2014) Improved Reweighting of Accelerated Molecular Dynamics Simulations for Free Energy Calculation. J. Chem. Theory Comput., 10, 2677-2689.
3. Woll, M. G., Qi, H., Turpoff, A., Zhang, N., Zhang, X., Chen, G., Li, C., Huang, S., Yang, T., Moon, Y. C., et al. (2016) Discovery and Optimization of Small Molecule Splicing Modifiers of Survival Motor Neuron 2 as a Treatment for Spinal Muscular Atrophy. J. Med. Chem., 59, 6070-6085.

### REFERENCE RELATED TO EXAMPLE 8

1. Hao, S. et al. Long-Term Modeling of SARS-CoV-2 Infection of In Vitro Cultured Polarized Human Airway Epithelium. MBio 11, (2020). DOI: 10.1128/mBio.02852-20.

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A compound of Formula I:

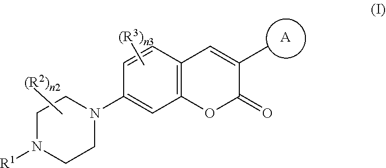

or a pharmaceutically acceptable salt and/or solvate thereof, wherein
   Ring A is C₆-C₁₀ aryl or 5 to 10 membered heteroaryl;
   R¹ is selected from H, C₁-C₆ alkyl, —CH₂CCH, or 2 to 15 membered heteroalkyl;
   each R² is independently halogen or C₁-C₆ alkyl;
   each R³ is independently selected from halogen, C₁-C₆ alkyl, or 2 to 6 membered heteroalkyl;
   n2 is 0, 1, 2, 3, 4, or 5; and
   n3 is 0, 1, 2, 3, or 4;
with the proviso that the compound is not
(S)-3-(6,8-dimethylimidazo[1,2-a]pyrazin-2-yl)-7-(4-ethyl-3-methylpiperazin-1-yl)-2H-chromen-2-one (C2);
(S)-3-(6,8-dimethylimidazo[1,2-a]pyrazin-2-yl)-7-(3-methylpiperazin-1-yl)-2H-chromen-2-one (C4);
tert-butyl (S)-(2-(4-(3-(6,8-dimethylimidazo[1,2-a]pyrazin-2-yl)-2-oxo-2H-chromen-7-yl)-2-methylpiperazin-1-yl)ethyl)carbamate (C6);
3-(3,4-dimethoxyphenyl)-7-(4-methylpiperazin-1-yl)-2H-chromen-2-one (C27);
3-(imidazo[1,2-a]pyridin-2-yl)-7-(piperazin-1-yl)-2H-chromen-2-one (C29);
3-(6-fluoroimidazo[1,2-a]pyridin-2-yl)-7-(piperazin-1-yl)-2H-chromen-2-one (C31);
3-(6-chloroimidazo[1,2-a]pyridin-2-yl)-7-(piperazin-1-yl)-2H-chromen-2-one (C33); or
7-(piperazin-1-yl)-3-(7-(trifluoromethyl)imidazo[1,2-a]pyridin-2-yl)-2H-chromen-2-one (C36).
   B. A compound of Formula II:

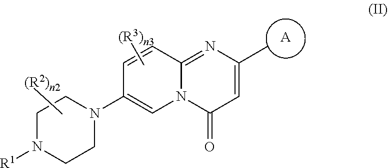

or a pharmaceutically acceptable salt and/or solvate thereof, wherein
   Ring A is C₆-C₁₀ aryl or 5 to 10 membered heteroaryl;
   R¹ is selected from H, C₁-C₆ alkyl, —CH₂CCH, or 2 to 15 membered heteroalkyl;
   each R² is independently halogen or C₁-C₆ alkyl;
   each R³ is independently selected from halogen, C₁-C₆ alkyl, or 2 to 6 membered heteroalkyl;
   n2 is 0, 1, 2, 3, 4, or 5; and
   n3 is 0, 1, 2, 3, or 4;
with the proviso that the compound is not
2-(4,6-dimethylpyrazolo[1,5-a]pyrazin-2-yl)-7-(4-ethylpiperazin-1-yl)-9-methyl-4H-pyrido[1,2-a]pyrimidin-4-one (C3); or
2-(8-fluoro-2-methylimidazo[1,2-a]pyridin-6-yl)-7-(4-methylpiperazin-1-yl)-4H-pyrido[1,2-a]pyrimidin-4-one (C5).
   C. The compound of Paragraph A or B, wherein Ring A is substituted or unsubstituted phenyl, substituted or unsubstituted naphthyl, substituted or unsubstituted quinolyl, substituted or unsubstituted imidazopyrazinyl, or substituted or unsubstituted imidazopyridinyl.
   D. The compound of any one of Paragraphs A-C, wherein each R¹ is independently H, —CH₃, or —CH₂CH₃.
   E. The compound of any one of Paragraphs A-C, wherein each R¹ is

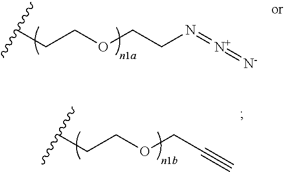

wherein n1a and n1b are independently selected from 1, 2, 3, 4, or 5.
F. The compound of any one of Paragraphs A-E, wherein each R² is independently hydrogen, —CH₃, or —CH₂CH₃.
G. The compound of any one of Paragraphs A-F, wherein each R³ is independently —F, —Cl, —Br, —CH₃, or —CH₂CH₃.
H. The compound of any one of Paragraphs A-F, wherein each R³ is independently —O(CH₂)₂NHC(O)O-t-butyl or —O(CH₂)₂NHC(O)OH.
I. The compound of any one of Paragraphs A-H, wherein Ring A is phenyl, naphthyl, quinolyl, imidazopyrazinyl, or imidazopyridinyl, each optionally substituted with 1 to 3 —F, —Cl, —Br, —CH₃, —CH₂CH₃, —CF₃, —OCH₃, —OCH₂CH₃, or —OCF₃.
J. The compound of any one of Paragraphs A-I, wherein n2 is 0 or 1.
K. The compound of any one of Paragraphs A-J, wherein n3 is 0 or 1.
L. The compound of Paragraph A selected from the group consisting of:

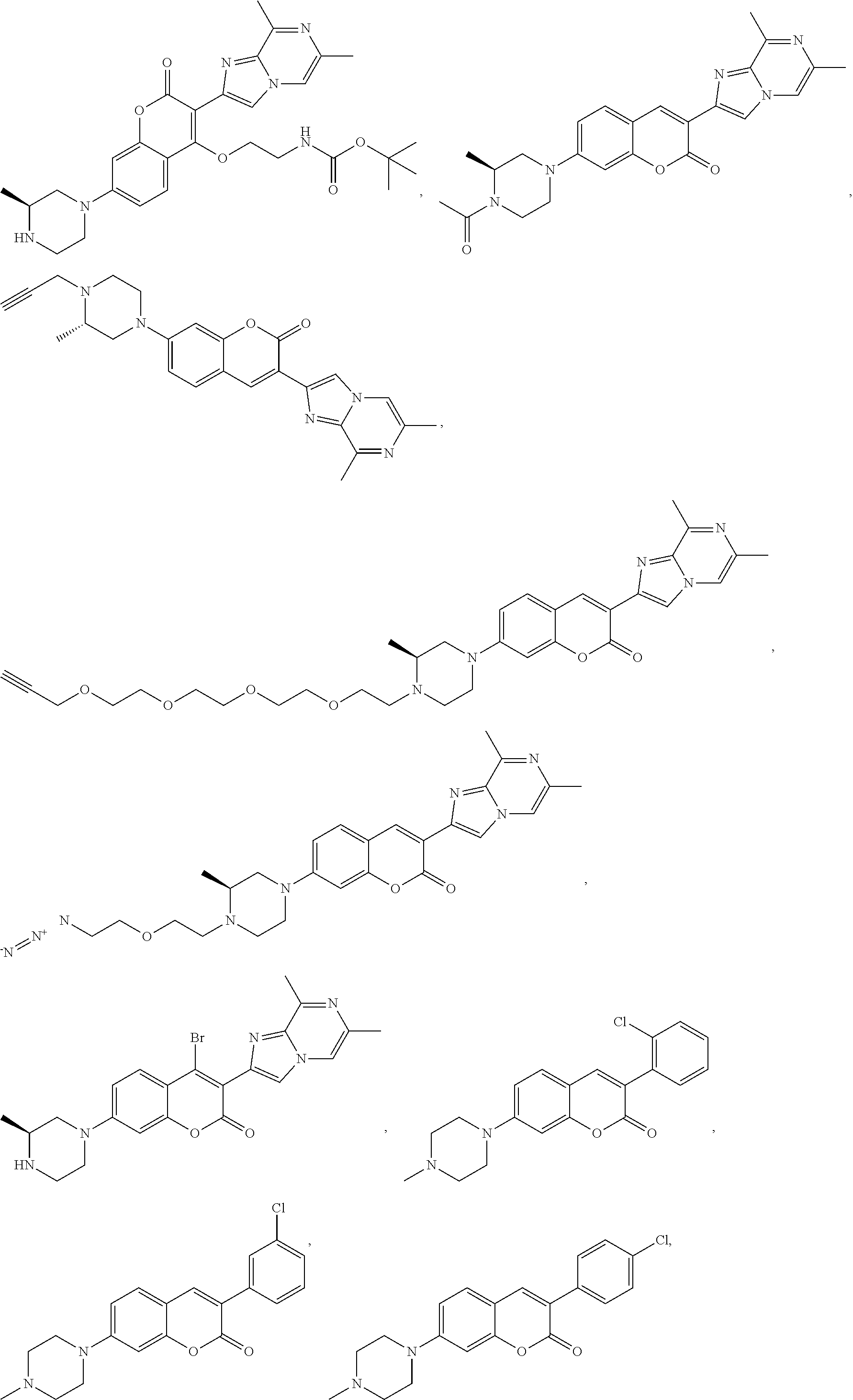

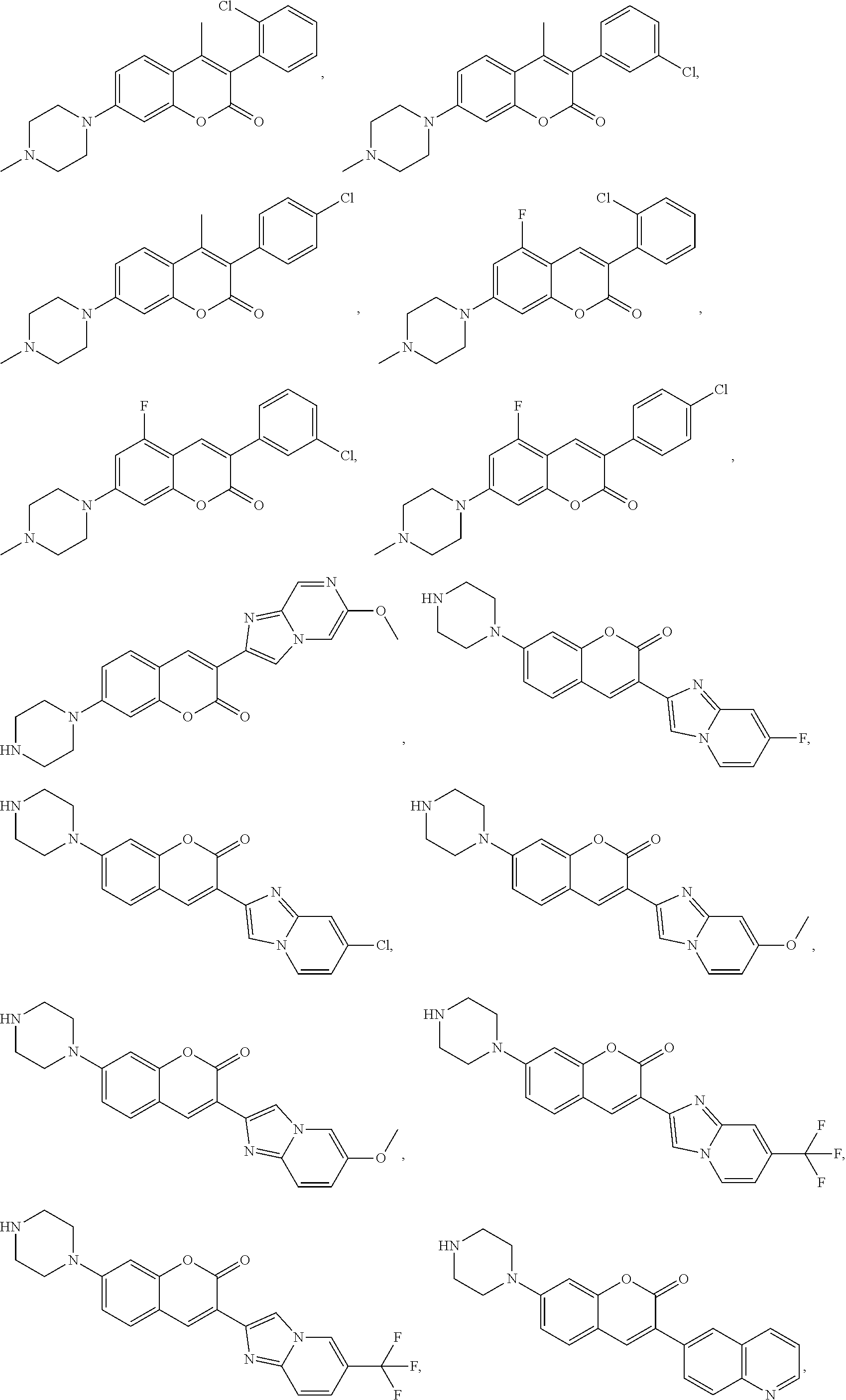

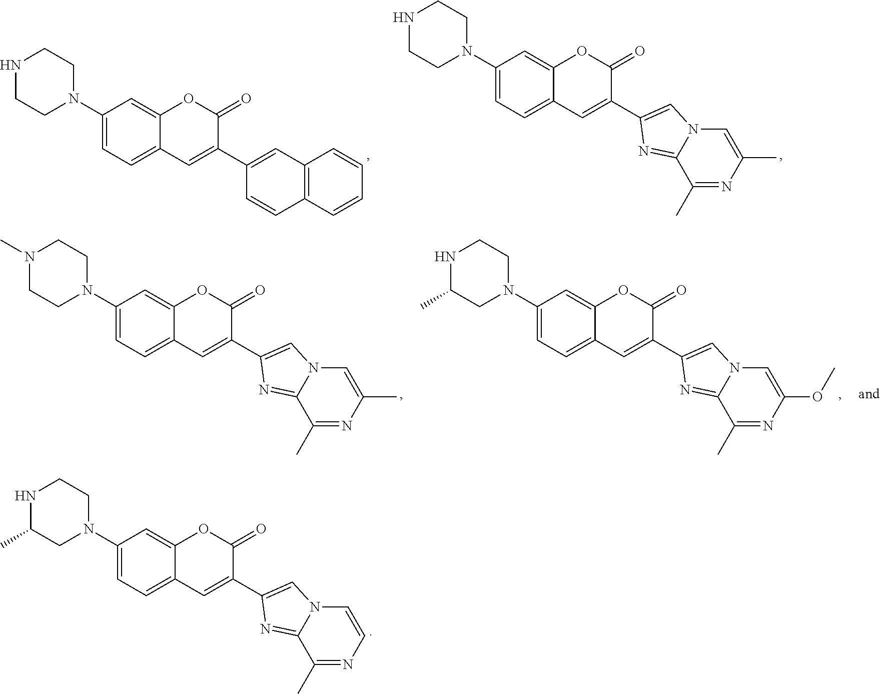

M. The compound of Paragraph B selected from the group consisting of:

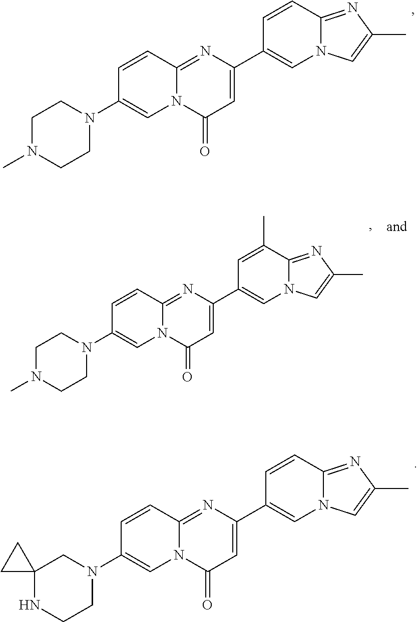

N. A compound of Formula III:

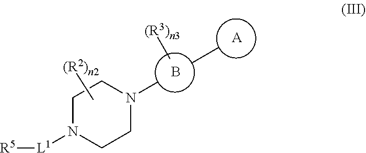

or a pharmaceutically acceptable salt and/or solvate thereof, wherein
   Ring A is C₆-C₁₀ aryl or 5 to 10 membered heteroaryl;
   Ring B is 5 to 10 membered heteroaryl;
L¹ is

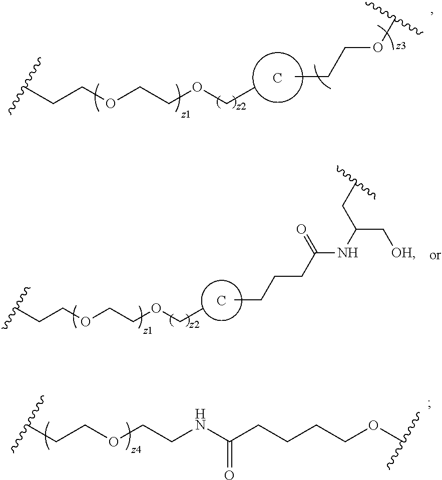

each R² is independently halogen or optionally substituted C₁-C₆ alkyl;
   each R³ is independently selected from halogen, optionally substituted C₁-C₆ alkyl, or optionally substituted 2 to 6 membered heteroalkyl;
   R⁵ is

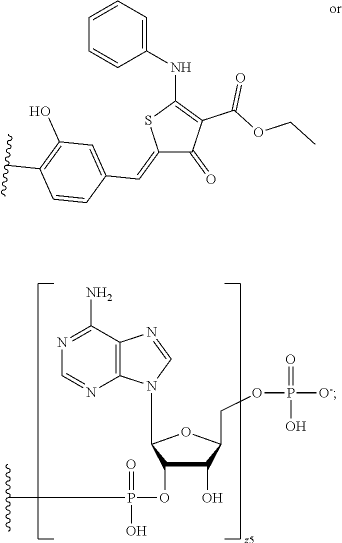

Ring C is a 5-membered heteroaryl;
   n2 is 0, 1, 2, 3, 4, or 5;
   n3 is 0, 1, 2, 3 or 4;
   z1, z3, and z4 are each independently 0, 1, 2, 3, or 4;
   z2 is independently at each occurrence 1 or 2; and
   z5 is 1, 2, 3, 4, 5, 6, 7, 8, 9, or 10.
O. The compound of Paragraph N, having chemical structure of Formula IIIa:

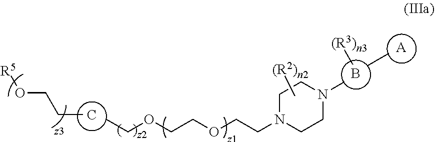

or a pharmaceutically acceptable salt and/or solvate thereof.
P. The compound of Paragraph N, having chemical structure of Formula IIIb:

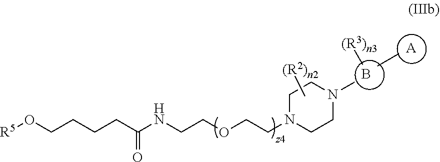

or a pharmaceutically acceptable salt and/or solvate thereof.
Q. The compound of Paragraph N, having chemical structure of Formula IIIc:

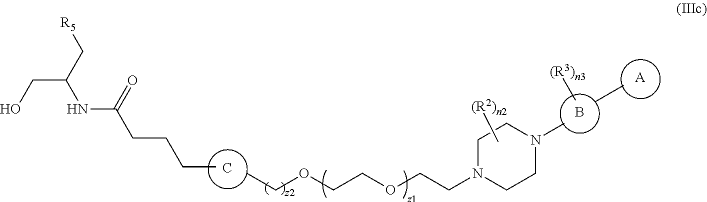

or a pharmaceutically acceptable salt and/or solvate thereof.
R. The compound of any one of Paragraphs N-S, wherein Ring B is selected from coumarin and pyridopyrimidone.
S. The compound of any one of Paragraphs N, O, or R, having the chemical structure of Formula IIIa′:

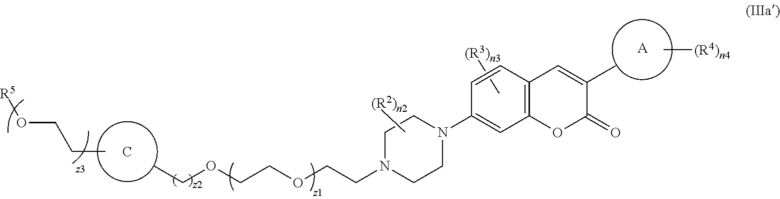

or a pharmaceutically acceptable salt and/or solvate thereof.
T. The compound of any one of Paragraphs N, O or R, having the chemical structure of Formula IIIb′:

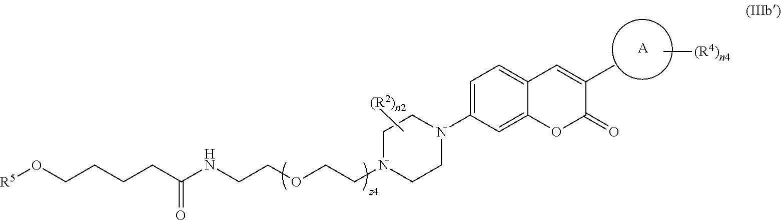

or a pharmaceutically acceptable salt and/or solvate thereof.
U. The compound of N or Q, having the chemical structure of Formula IIIc′:

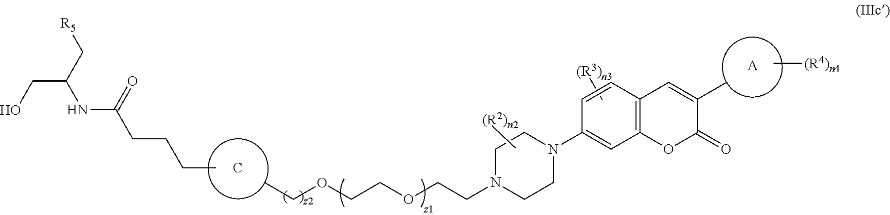

or a pharmaceutically acceptable salt and/or solvate thereof.
V. The compound of any one of Paragraphs N, O, Q, R, S or U, wherein Ring C is 1,2,3-triazinyl.
W. The compound of any one of Paragraphs N-V, wherein Ring A is substituted or unsubstituted phenyl, naphthyl, quinolyl, imidazopyrazinyl, or imidazopyridinyl.
X. The compound of any one of Paragraphs N-W, wherein each R² is independently hydrogen, —CH₃, or —CH₂CH₃.
Y. The compound of any one of Paragraphs N-X, wherein each R³ is independently —F, —Cl, —Br, CH₃, or CH₂CH₃.
Z. The compound of any one of Paragraphs N-W, wherein each R³ is independently —O(CH₂)₂NHC(O)O-t-butyl or —O(CH₂)₂NHC(O)OH.
AA. The compound of any one of Paragraphs N-Z, wherein Ring A is phenyl, naphthyl, quinolyl, imidazopyrazinyl, or imidazopyridinyl each optionally substituted with 1 to 3 —F, —Cl, —Br, —CH₃, —CH₂CH₃, —CF₃, —OCH₃, —OCH₂CH₃, or —OCF₃.
AB. The compound of any one of Paragraphs N-AA, wherein n2 is 0 or 1.
AC. The compound of any one of Paragraphs N-AB, wherein n3 is 0 or 1.
AD. A compound selected from the group consisting of:

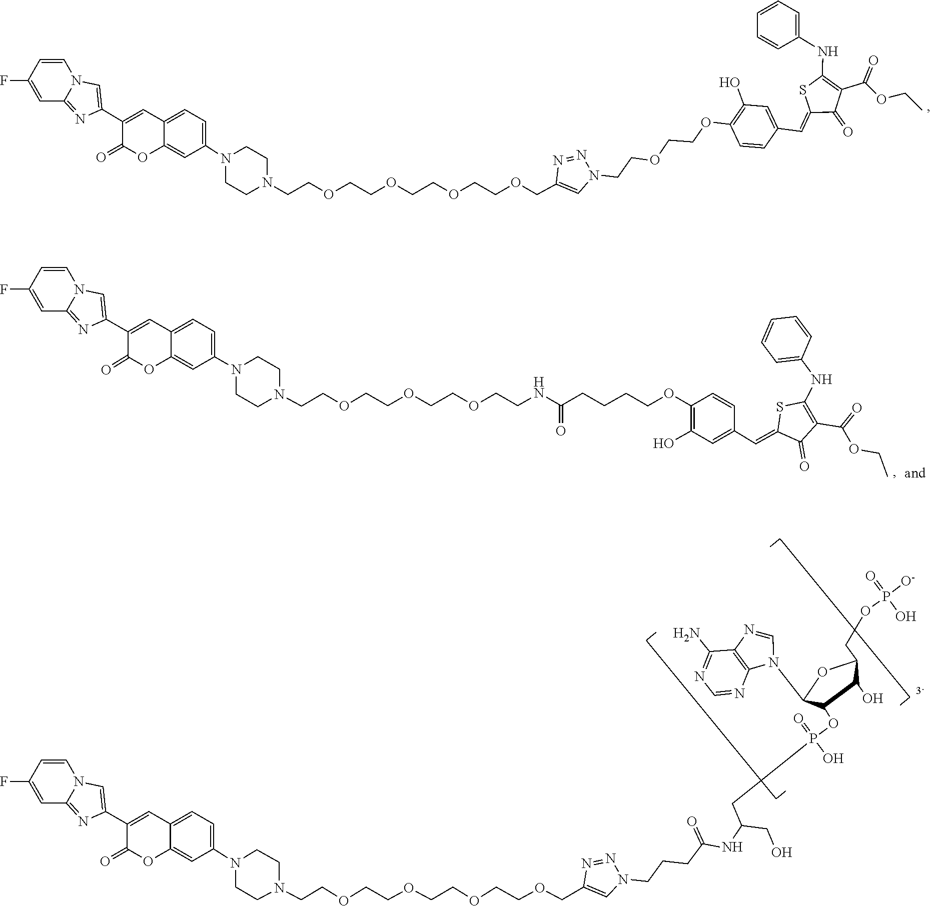

AE. A pharmaceutical composition comprising the compound of any one of Paragraphs A-AD, or the stereoisomer or the tautomer thereof, or the pharmaceutically acceptable salt thereof, and at least one pharmaceutically acceptable excipient.
AF. A method of treating a disorder or disease in a subject in need thereof, the method comprising administering to the subject a therapeutically effective amount of the compound of any one of Paragraphs A-AD, or the stereoisomer or the tautomer thereof, or the pharmaceutically acceptable salt thereof, or a therapeutically effective amount of the pharmaceutical composition of Paragraph AE, wherein the disorder or disease is a viral disorder or disease.
AG. The method of Paragraph AF, wherein the disease is COVID-19.
AH. The method of Paragraph AG, wherein the method comprises an additional therapeutic agent.
AI. The method of Paragraph AH, wherein the additional therapeutic agent is remdesivir.

Other embodiments are set forth in the following claims, along with the full scope of equivalents to which such claims are entitled.

1. A compound of Formula I: 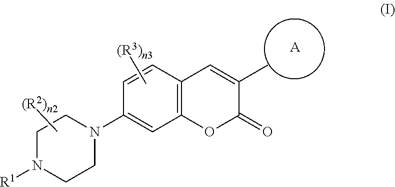
or a pharmaceutically acceptable salt and/or solvate thereof, wherein
Ring A is phenyl substituted with 1 to 3 —F, —Cl, —Br, —CH₃, —CH₂CH₃, —CF₃, —OCH₃, —OCH₂CH₃, or —OCF₃; naphthyl optionally substituted with 1 to 3 —F, —Cl, —Br, —CH₃, —CH₂CH₃, —CF₃, —OCH₃, —OCH₂CH₃, or —OCF₃; quinolyl optionally substituted with 1 to 3 —F, —Cl, —Br, —CH₃, —CH₂CH₃, —CF₃, —OCH₃, —OCH₂CH₃, or —OCF₃; imidazopyrazinyl, optionally substituted with 1 to 3 —F, —Cl, —Br, —CH₃, —CH₂CH₃, —CF₃, —OCH₃, —OCH₂CH₃, or —OCF₃; or imidazopyridinyl optionally substituted with 1 to 3 —F, —Cl, —Br, —CH₃, —CH₂CH₃, —CF₃, —OCH₃, —OCH₂CH₃, or —OCF₃;
R¹ is selected from H, C₁-C₆ alkyl, —CH₂CCH, or 2 to 15 membered heteroalkyl;
each R² is independently halogen or C₁-C₆ alkyl;
each R³ is independently selected from halogen, C₁-C₆ alkyl, or 2 to 6 membered heteroalkyl;
n2 is 0, 1, 2, 3, 4, or 5; and
n3 is 0, 1, 2, 3, or 4;
with the proviso that the compound is not
(S)-3-(6,8-dimethylimidazo[1,2-a]pyrazin-2-yl)-7-(4-ethyl-3-methylpiperazin-1-yl)-2H-chromen-2-one (C2);
(S)-3-(6,8-dimethylimidazo[1,2-a]pyrazin-2-yl)-7-(3-methylpiperazin-1-yl)-2H-chromen-2-one (C4);
tert-butyl (S)-(2-(4-(3-(6,8-dimethylimidazo[1,2-a]pyrazin-2-yl)-2-oxo-2H-chromen-7-yl)-2-methylpiperazin-1-yl)ethyl)carbamate (C6);
3-(3,4-dimethoxyphenyl)-7-(4-methylpiperazin-1-yl)-2H-chromen-2-one (C27);
3-(imidazo[1,2-a]pyridin-2-yl)-7-(piperazin-1-yl)-2H-chromen-2-one (C29);
3-(6-fluoroimidazo[1,2-a]pyridin-2-yl)-7-(piperazin-1-yl)-2H-chromen-2-one (C31);
3-(6-chloroimidazo[1,2-a]pyridin-2-yl)-7-(piperazin-1-yl)-2H-chromen-2-one (C33); or
7-(piperazin-1-yl)-3-(7-(trifluoromethyl)imidazo[1,2-a]pyridin-2-yl)-2H-chromen-2-one (C36).

2. A compound of Formula II: 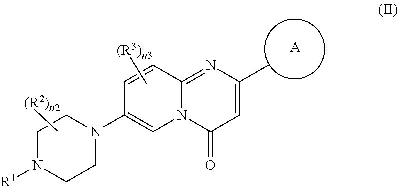
or a pharmaceutically acceptable salt and/or solvate thereof, wherein
Ring A is C₆-C₁₀ aryl or 5 to 10 membered heteroaryl;
R¹ is selected from H, C₁-C₆ alkyl, —CH₂CCH, or 2 to 15 membered heteroalkyl;
each R² is independently halogen or C₁-C₆ alkyl;
each R³ is independently selected from halogen, C₁-C₆ alkyl, or 2 to 6 membered heteroalkyl;
n2 is 0, 1, 2, 3, 4, or 5; and
n3 is 0, 1, 2, 3, or 4;
with the proviso that the compound is not
2-(4,6-dimethylpyrazolo[1,5-a]pyrazin-2-yl)-7-(4-ethylpiperazin-1-yl)-9-methyl-4H-pyrido[1,2-a]pyrimidin-4-one (C3); or
2-(8-fluoro-2-methylimidazo[1,2-a]pyridin-6-yl)-7-(4-methylpiperazin-1-yl)-4H-pyrido[1,2-a]pyrimidin-4-one (C5).

3.-4. (canceled)

5. The compound of claim 1, wherein each R¹ is 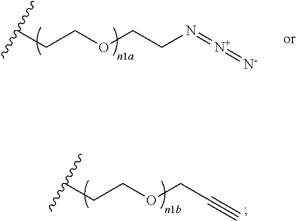
wherein n1a and n1b are independently selected from 1, 2, 3, 4, or 5.

6-11. (canceled)

12. The compound of claim 1 selected from the group consisting of: 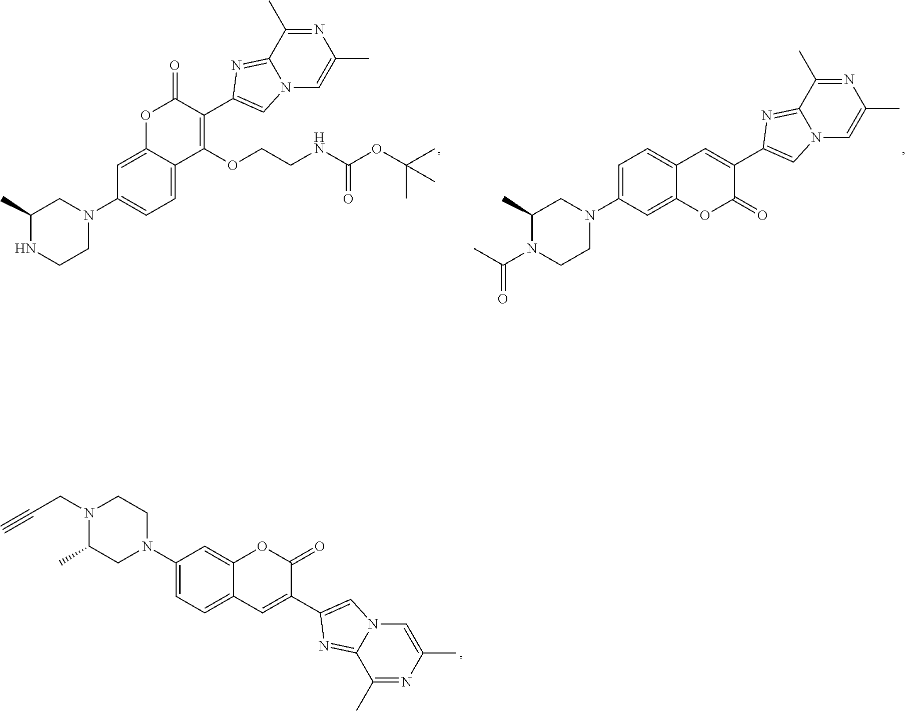 [SMILES: Cc1cn2cc(-c3c(OCCNC(=O)OC(C)(C)C)c4ccc(N5CCN[C@@H](C)C5)cc4oc3=O)nc2c(C)n1 ; CC(=O)N1CCN(c2ccc3cc(-c4cn5cc(C)nc(C)c5n4)c(=O)oc3c2)C[C@@H]1C ; C#CCN1CCN(c2ccc3cc(-c4cn5cc(C)nc(C)c5n4)c(=O)oc3c2)C[C@@H]1C] 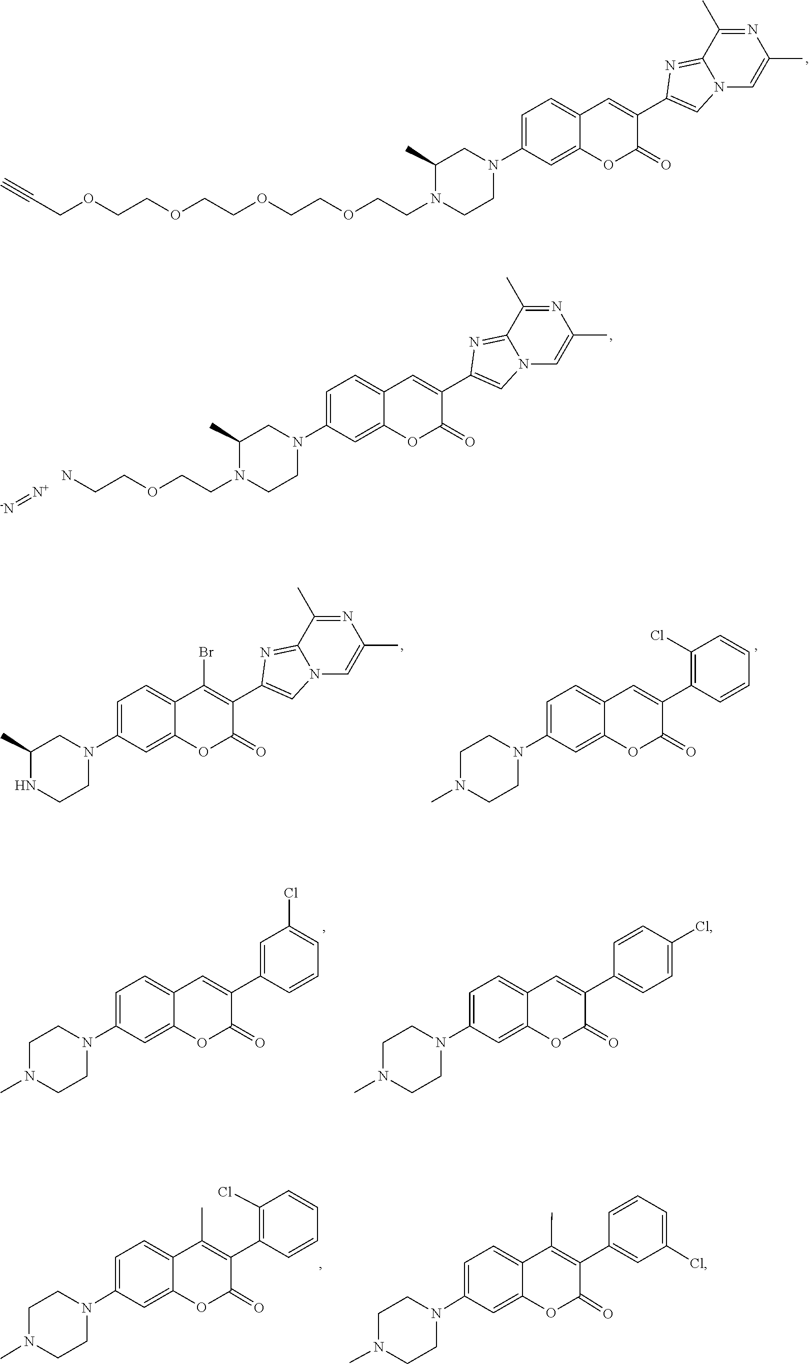 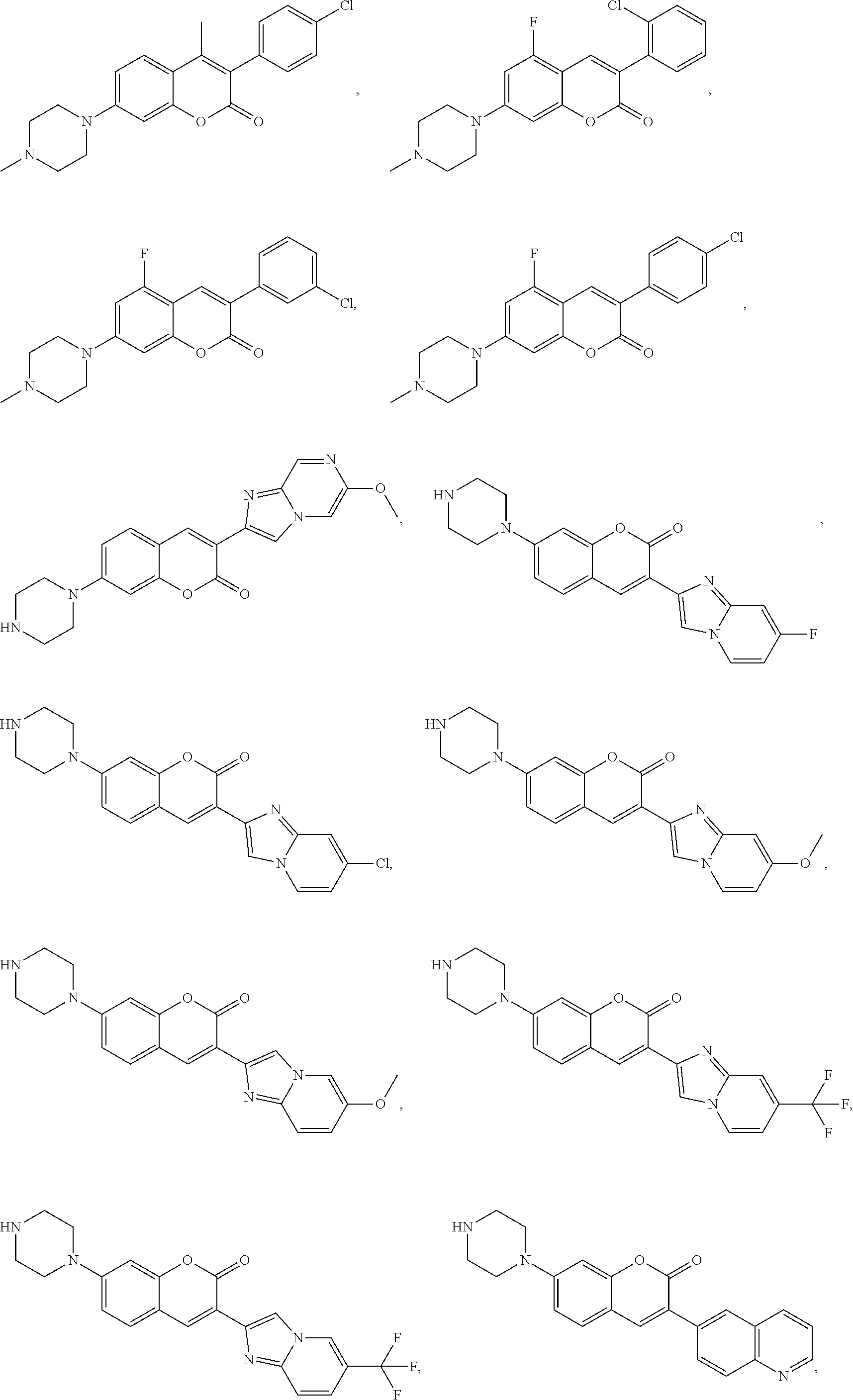 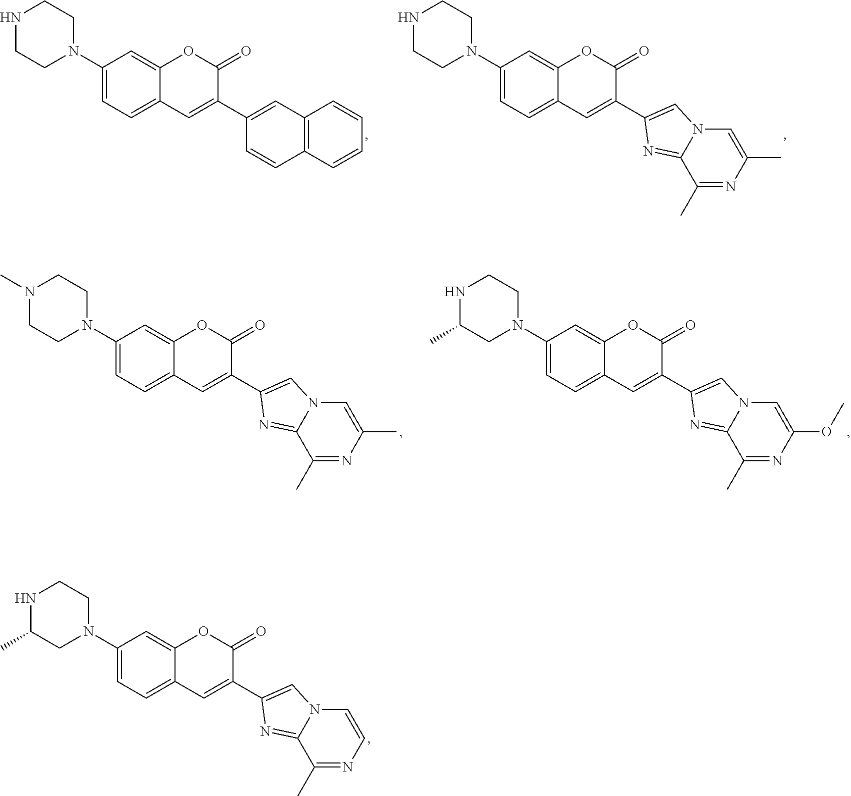
and a pharmaceutically acceptable salt of any one thereof.

13. (canceled)

14. A compound of Formula III: 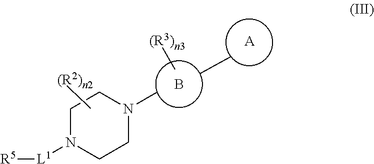
or a pharmaceutically acceptable salt and/or solvate thereof, wherein
Ring A is C₆-C₁₀ aryl or 5 to 10 membered heteroaryl;
Ring B is 5 to 10 membered heteroaryl;
L¹ is 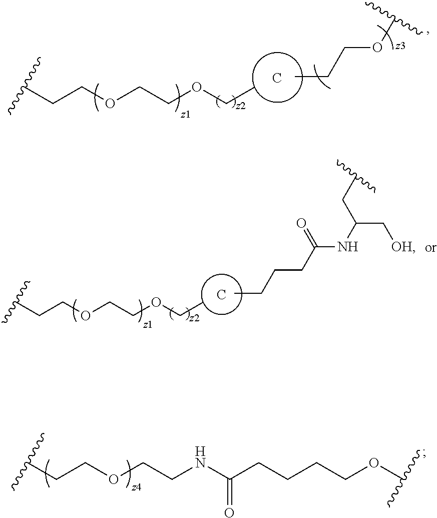
each R² is independently halogen or optionally substituted C₁-C₆ alkyl;
each R³ is independently selected from halogen, optionally substituted C₁-C₆ alkyl, or optionally substituted 2 to 6 membered heteroalkyl;
R⁵ is 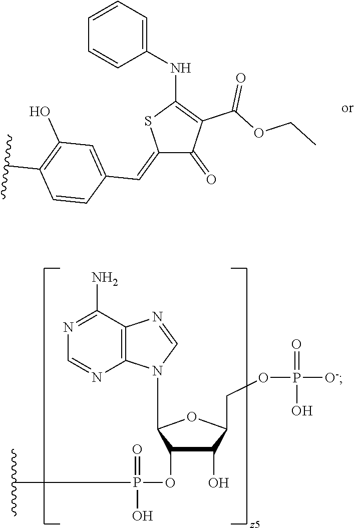
Ring C is a 5-membered heteroaryl;
n2 is 0, 1, 2, 3, 4, or 5;
n3 is 0, 1, 2, 3 or 4;
z1, z3, and z4 are each independently 0, 1, 2, 3, or 4;
z2 is independently at each occurrence 1 or 2; and
z5 is 1, 2, 3, 4, 5, 6, 7, 8, 9, or 10.

15. The compound of claim 14, having chemical structure of Formula IIIa: 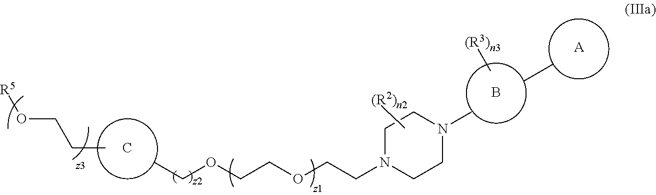
or a pharmaceutically acceptable salt and/or solvate thereof.

16. The compound of claim 14, having chemical structure of Formula IIIb: 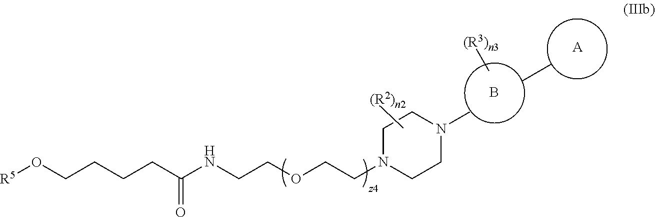
or a pharmaceutically acceptable salt and/or solvate thereof.

17. The compound of claim 14, having chemical structure of Formula IIIc: 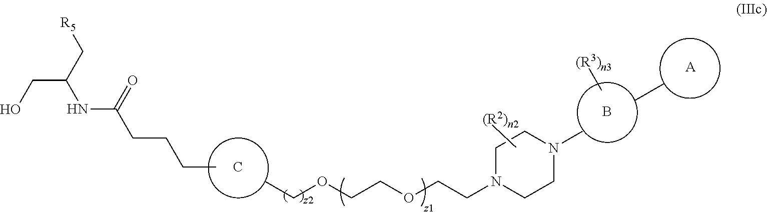
or a pharmaceutically acceptable salt and/or solvate thereof.

18. (canceled)

19. The compound of claim 14, having the chemical structure of Formula IIIa′: 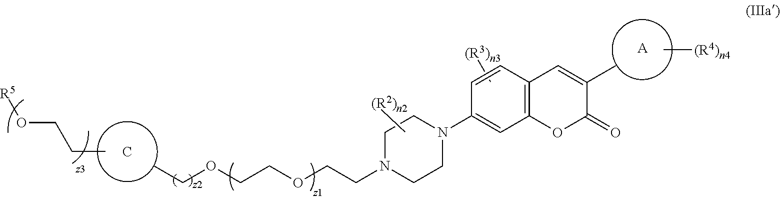
or a pharmaceutically acceptable salt and/or solvate thereof.

20. The compound of claim 14, having the chemical structure of Formula IIIb′: 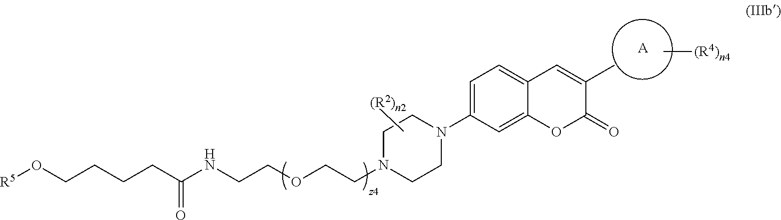
or a pharmaceutically acceptable salt and/or solvate thereof.

21. The compound of claim 14, having the chemical structure of Formula IIIc′: 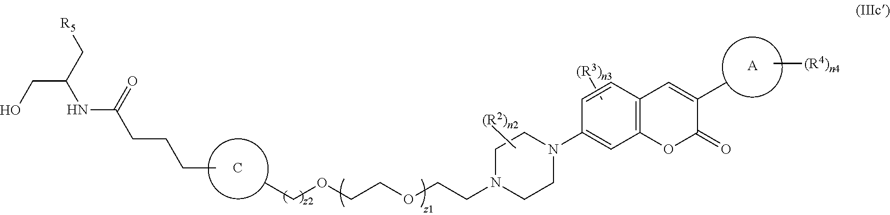
or a pharmaceutically acceptable salt and/or solvate thereof.

22. The compound of claim 14, wherein Ring C is 1,2,3-triazinyl.

23. The compound of claim 14, wherein Ring A is substituted or unsubstituted phenyl, naphthyl, quinolyl, imidazopyrazinyl, or imidazopyridinyl.

24. The compound of claim 14, wherein each R² is independently halogen, —CH₃, or —CH₂CH₃.

25. The compound of claim 14, wherein each R³ is independently —F, —Cl, —Br, —CH₃, or —CH₂CH₃.

26. The compound of claim 14, wherein each R³ is independently —O(CH₂)₂NHC(O)O-t-butyl or —O(CH₂)₂NHC(O)OH.

27. The compound of claim 14, wherein Ring A is phenyl, naphthyl, quinolyl, imidazopyrazinyl, or imidazopyridinyl each optionally substituted with 1 to 3 —F, —Cl, —Br, —CH₃, —CH₂CH₃, —CF₃, —OCH₃, —OCH₂CH₃, or —OCF₃.

28.-29. (canceled)

30. A compound of claim 14 selected from the group consisting of: 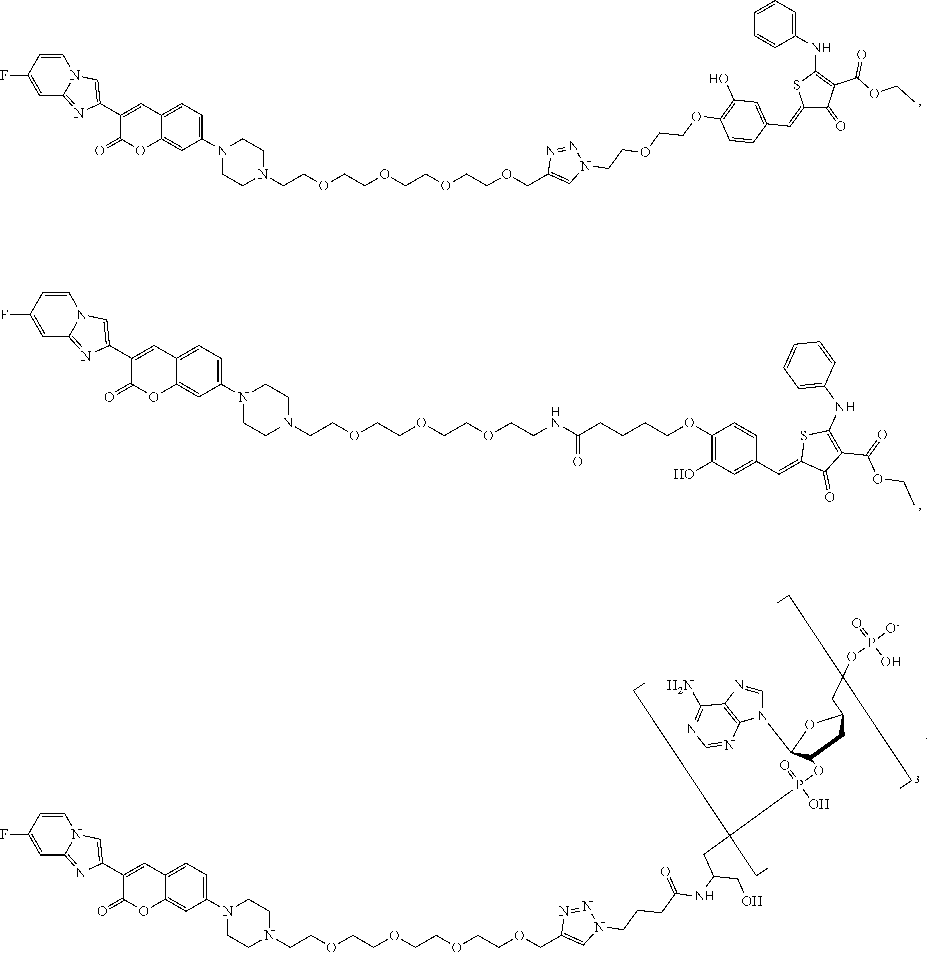
and a pharmaceutically acceptable salt of any one thereof.

31. A compound of Formula IV: 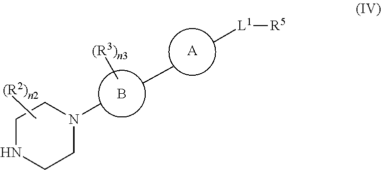
or a pharmaceutically acceptable salt and/or solvate thereof, wherein
Ring A is C₆-C₁₀ aryl or 5 to 10 membered heteroaryl;
Ring B is 5 to 10 membered heteroaryl;
L¹ is 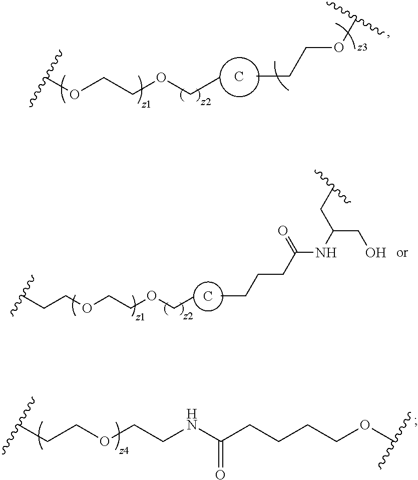
each R² is independently halogen or optionally substituted C₁-C₆ alkyl;
each R³ is independently selected from halogen, optionally substituted C₁-C₆ alkyl, or optionally substituted 2 to 6 membered heteroalkyl;
R⁵ is 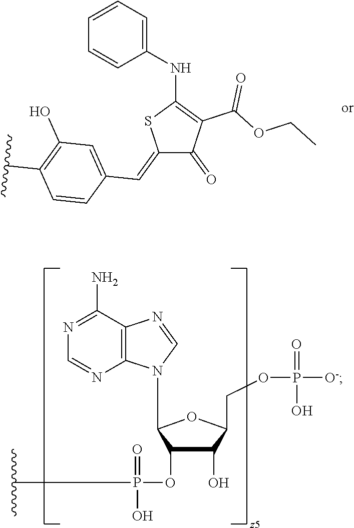
Ring C is a 5-membered heteroaryl;
n2 is 0, 1, 2, 3, 4, or 5;
n3 is 0, 1, 2, 3 or 4;
z1, z3, and z4 are each independently 0, 1, 2, 3, or 4;
z2 is independently at each occurrence 1 or 2; and
z5 is 1, 2, 3, 4, 5, 6, 7, 8, 9, or 10.

32.-47. (canceled)

48. A method of treating a disorder or disease in a subject in need thereof, the method comprising administering to the subject a therapeutically effective amount of the compound of claim 1, or the stereoisomer or the tautomer thereof, or the pharmaceutically acceptable salt thereof, wherein the disorder or disease is a viral disorder or disease.

49-51. (canceled)
